(19) United States
(12) Patent Application Publication
(10) Pub. No.: US 2014/0273015 A1
(43) Pub. Date: Sep. 18, 2014

(54) BINDING COMPOUNDS TO HUMAN Beta 1-ADRENORECEPTOR (Beta 1-AR) AND THEIR USE IN MEASUREMENT OF AUTO-ANTI- Beta 1-AR ANTIBODIES

(71) Applicants: Hans-Peter Holthoff, Penzberg (DE); Martin Ungerer, Martinsried (DE); Stefan Zeibig, Grafelfing (DE); Martin J. Lohse, Wfuzburg (DE); Roland Jahns, Wfuzburg (DE); Valerie Jahns, Wfuzburg (DE)

(72) Inventors: Hans-Peter Holthoff, Penzberg (DE); Martin Ungerer, Martinsried (DE); Stefan Zeibig, Grafelfing (DE); Martin J. Lohse, Wfuzburg (DE); Roland Jahns, Wfuzburg (DE); Valerie Jahns, Wfuzburg (DE)

(73) Assignee: CORIM- MUN GMBH, Neuss (DE)

(21) Appl. No.: 14/124,649

(22) PCT Filed: Jun. 6, 2012
(86) PCT No.: PCT/EP2012/060776

§ 371 Date: May 7, 2014

(30) Foreign Application Priority Data
Jun. 10, 2011 (EP) 11169621.7

(51) Int. Cl. C07K 16/28; G01N 33/68; G01N 33/74

(52) U.S. Cl. CPC C07K 16/2869; G01N 33/74; G01N 33/6893
USPC 435/7.21; 530/387.9; 530/387.3; 536/23.53; 435/320.1; 435/331; 435/69.6; 436/501; 435/7.92

(57) ABSTRACT

The present disclosure relates to binding compounds/antibodies that bind to the second extracellular loop of the human β1-adrenoreceptor (β1-AR-ECII) that are produced by/obtainable from a host cell/hybridoma with a deposit number selected from the group consisting of DSM ACC3121, DSM ACC3174, DSM ACC3175, DSM ACC3176 and DSM AC-C3177. The binding compounds/antibodies are particularly useful in determination of auto-anti-β1-AR antibodies in an in vitro cell based assay system in order to characterize and to identify auto-antibodies directed against the β1-AR-ECII in a biological sample. Further aspects of the disclosure are nucleic acid molecules encoding said binding compounds/antibodies, vectors, host cells, methods for producing the binding compounds/antibodies of the disclosure as well as a kit comprising the binding compounds/antibodies of the present disclosure.

### FIELD

Embodiments of the present invention relate to a binding compound that binds to the second extracellular loop of the human β1-adrenoreceptor (β1-AR-ECII) and the invention relates in particular to the binding compound/antibody that is produced by and/or obtainable from the host cell/hybridoma, with the deposit number DSM ACC3121. The invention also relates to antibodies binding to the second extracellular loop of the human β1-adrenoreceptor that are produced by/obtainable from a host cell hybridoma with a deposit number selected from the group consisting of DSM ACC3174, DSM ACC3175, DSM ACC3176 and DSM ACC3177.

### BACKGROUND

Progressive cardiac dilatation and pump failure of unknown etiology has been termed “idiopathic” dilated cardiomyopathy (DCM) (Richardson, Circulation 93 (1996), 841-842). DCM represents one of the main causes of severe heart failure with an annual incidence of up to 100 patients and a prevalence of 300-400 patients per million (AHA report 2007). At present the large majority of DCM is thought to arise from an initial (mostly viral) infection leading to acute myocarditis which upon activation of the immune system may progress to (chronic) autoimmune myocarditis resulting in cardiac dilatation and severe congestive heart failure. The severe congestive heart failure occurs particularly, when associated (a) with the development of auto-antibodies against distinct myocyte sarcolemmal or membrane proteins which are essential for cardiac function (Freedman, J. Clin. Invest. 113 (2004), 1379-1382; Jahns, Trends Cardiovasc Med 16 (2006), 20-24), or (b) with chronic inflammation of the myocardium and viral persistence (Kühl, Circulation 112 (2005), 1965-1970). These findings are further strengthened by the fact, that patients suffering from DCM often have alterations in both cellular and humoral immunity (Jahns, Trends Cardiovasc Med 16 (2006), 20-24, Limas Circulation 95 (1997), 1979-1980, Luppi, Circulation 98 (1998), 777-785, Mahrholdt, Circulation 114 (2006), 1581-1590). In the context of their humoral response a substantial number of DCM patients have been found to develop auto-antibodies to various cardiac antigens. Among them only a subgroup of auto-antibodies directed against the second extracellular loop of the (human) beta1-adrenoreceptor/beta1-adrenergic receptor (β1-AR) has been shown to exert agonist-like actions on human beta adrenoreceptors developing cardiac dilatation and dysfunction.

### SUMMARY

The present invention relates to a binding compound that binds to the second extracellular loop of the human β1-adrenoreceptor (β1-AR-ECII) and the invention relates in particular to the binding compound/antibody that is produced by and/or obtainable from the host cell/hybridoma, with the deposit number DSM ACC3121. The invention also relates to antibodies binding to the second extracellular loop of the human β1-adrenoreceptor that are produced by/obtainable from a host cell hybridoma with a deposit number selected from the group consisting of DSM ACC3174, DSM ACC3175, DSM ACC3176 and DSM ACC3177. The binding compounds/antibodies of the present invention are particularly useful in determination of auto-anti-β1-AR antibodies in in vitro assays in order to characterize and identify auto-antibodies directed against the β1-AR-ECII in a biological sample in a cellular ELISA assay that is based on an over-expression of human β1-adrenoreceptor (β1-AR) in SF9 cells by baculovirus. Furthermore, nucleic acid molecules encoding said binding compounds/antibodies as well as vectors and host cells comprising the same are described in the present invention. The present invention also provides methods for producing the binding compounds/antibodies of the invention. In addition, a method for identifying a patient having or being at risk of developing a disease associated with human β1-adrenoreceptor (β1-AR), like idiopathic dilated cardiomyopathy (DCM) or ischaemic cardiomyopathy (ICM), is described. The present invention also relates to diagnostic means, methods and uses taking advantage of the binding compounds/antibodies of the invention for detecting molecules/compounds in a biological sample like auto-anti-β1 AR (β1-adrenoreceptor/β1-adrenergic receptor) antibodies. Finally, a kit comprising the compounds of the present invention is described.

### BRIEF DESCRIPTION OF THE DRAWINGS

The following drawings form part of the present specification and are included to further demonstrate certain embodiments of the present invention. The embodiments may be better understood by reference to one or more of these drawings in combination with the detailed description of specific embodiments presented herein.

FIG. 1: ELISA binding assay using the 26-meric peptide (His-Trp-Trp-Arg-Ala-Glu-Ser-Asp-Glu-Ala-Arg-Arg-Cys-Tyr-Asn-Asp-Pro-Lys-Cys-Cys-Asp-Phe-Val-Thr-Asn-Arg (SEQ ID NO:17)).

FIG. 2: ELISA-based determination of the affinity of different clones of monoclonal murine antibody to second extracellular domain of the human β1-adrenoceptor (β1-ECII AR).

FIG. 3: Overview of the concentration with half maximal efficacy response (EC50)-values of the five mouse monoclonal antibodies produced by the hybridoma clones 23-6-7, 47-12-9, 50-1-5, 55-3-10 and 28-2-7 binding to human β1-AR.

FIG. 4: Binding characteristics of various antibodies obtained from mouse, rat and goat respectively, to human β1-AR, which were overexpressed in SF9 cells (in presence or absence of 0.1% TWEEN 20).

FIG. 5: Comparison of the ELISA-based determination of the affinity of various antibodies to anti-β1-AR ECII in presence or absence of TWEEN 20.

FIG. 6: Competition of the monoclonal murine (23-6-7) antibody binding to the second β1-AR by cyclo (Ala-Asp-Glu-Ala-Arg-Arg-Cys-Tyr-Asn-Asp-Pro-Lys-Cys-Ser-Asp-Phe-Val-Gln) referring to SEQ ID NO:16.

FIG. 7: Inhibition values above cut-off of sera taken from individual idiopathic dilated cardiomyopathy (DCM) patients.

FIG. 8: Inhibition values above cut-off of sera taken from healthy volunteers.

FIG. 9: Determination of the percentage of positive auto-anti-β1-AR antibodies binding to the second extracellular loop of the human β1-adrenoreceptor (β1-AR-ECII) in DCM patients or healthy controls.

FIG. 10: Principle of the ELISA measurement of human anti-β1-AR (human β1-adrenoreceptor) antibodies via competition of the monoclonal mouse anti-β1-AR-ECII antibody 23-6-7.

FIG. 11: Binding affinity of the mouse monoclonal antibody that is obtainable from the host cell/hybridoma with the deposit number DSM ACC3121

FIG. 12: Measurement of cAMP levels by Epac-FRET in human embryonic kidney HEK293 cells stably expressing human β1-ARs.

FIG. 13: Competition of the binding of the mouse monoclonal anti-antibody 23-6-7 that is obtainable from the host cell/hybridoma with the deposit number DSM ACC3121 by polyclonal goat anti-β1-AR antibodies.

FIGS. 14A-14C: Determination of the auto-anti-β1-AR antibodies binding to the second extracellular loop of the human β1-adrenoreceptor (β1-AR-ECII) in DCM patients or healthy controls (Figures (A) and (B)) and ICM (Figure (C)) patients.

FIG. 15: Comparison of the inhibitory effect of unaltered sera and the respective antibody-depleted serum fractions.

### DETAILED DESCRIPTION

Progressive cardiac dilatation and pump failure of unknown etiology has been termed “idiopathic” dilated cardiomyopathy (DCM) (Richardson, Circulation 93 (1996), 841-842). DCM represents one of the main causes of severe heart failure with an annual incidence of up to 100 patients and a prevalence of 300-400 patients per million (AHA report 2007). At present the large majority of DCM is thought to arise from an initial (mostly viral) infection leading to acute myocarditis which upon activation of the immune system may progress to (chronic) autoimmune myocarditis resulting in cardiac dilatation and severe congestive heart failure. The severe congestive heart failure occurs particularly, when associated (a) with the development of auto-antibodies against distinct myocyte sarcolemmal or membrane proteins which are essential for cardiac function (Freedman, J. Clin. Invest. 113 (2004), 1379-1382; Jahns, Trends Cardiovasc Med 16 (2006), 20-24), or (b) with chronic inflammation of the myocardium and viral persistence (Kühl, Circulation 112 (2005), 1965-1970). These findings are further strengthened by the fact, that patients suffering from DCM often have alterations in both cellular and humoral immunity (Jahns, Trends Cardiovasc Med 16 (2006), 20-24, Limas Circulation 95 (1997), 1979-1980, Luppi, Circulation 98 (1998), 777-785, Mahrholdt, Circulation 114 (2006), 1581-1590). In the context of their humoral response a substantial number of DCM patients have been found to develop auto-antibodies to various cardiac antigens. Among them only a subgroup of auto-antibodies directed against the second extracellular loop of the (human) beta1-adrenoreceptor/beta1-adrenergic receptor (β1-AR) has been shown to exert agonist-like actions on human beta adrenoreceptors developing cardiac dilatation and dysfunction.

Accordingly, evidence has accumulated from both animal and patient-based studies that functionally active auto-antibodies targeting the (human) beta1-adrenoreceptor (β1-AR) play an important role in the development and clinical course of progressive cardiac dilatation and failure (Wallukat, Eur. Heart J. 12 (1991), 178-181; Magnusson, Circulation 89 (1994), 2760-2767; Jahns, Circulation 99 (1999), 649-654 and Iwata, J. Am. Coll. Cardiol. 37 (2001), 418-424). Beta1-adrenorecpetors (β1-ARs) are G protein-coupled receptors that trigger signalling via adenylate cyclase, cyclic adenosine monophosphate (cAMP), and PKA. This signalling pathway regulates the sarcoplasmic calcium concentration and increases cardiomyocyte contractility.

During recent years, it has been independently demonstrated by various groups that a relevant class of auto-antibodies bind to the second loop of the β1-AR and recognize a native receptor conformation (Jahns Circulation 99 (1999), 649-654; Iwata J Am Coll Cardiol 37 (2001), 418-424; Nikolaev J Am Coll Cardiol 50 (2007), 423-443 and Elies J Immunol. 157 (1996), 4203-4211). Such conformational anti-β1AR-(ECII) antibodies have been shown to be functionally active and appear to be capable of stimulating intracellular cAMP production (Jahns, Circulation 99 (1999), 649-654 and Nikolaev, Am Coll Cardiol 50 (2007), 423-443). Moreover, only those anti-β1AR auto-antibodies that target the second extracellular loop (β1-AR-ECII) appear to be functionally active. In contrast, antibodies directed against the amino- or carboxy terminus of the receptor protein exert no biological effects (Wallukat, Eur Heart J 12 (1991), 178-181; Elies, J Immunol. 157 (1996), 4203-4211 and Borda, Clin Exp Immunol. 57 (1984), 679-686).

Regarding functionally active auto-antibodies which are directed against the second extracellular loop of the human β1-adrenoreceptor (β1-AR) it has been demonstrated that their prevalence is almost negligible in healthy individuals (<1%) provided that a screening procedure based on cell-systems presenting the target (i.e., the (human) β1-AR) in its natural conformation is used (Jahns, Circulation 99 (1999), 649-654). By employing this screening method, occurrence of auto-anti-β1-AR antibodies could also be excluded in patients with chronic valvular or hypertensive heart disease (Jahns, J. Am. Coll. Cardiol 34 (1999), 1545-1551). In contrast auto-antibodies directed against the second extracellular loop of the β1-AR are well known and found in approximately 30%-50% of patients with DCM, depending on the respective study or screening method. A smaller percentage of patients with ischemic cardiomyopathy, approximately 10%-20%, were judged to be anti β1-AR antibody-positive (Störk, Am. Heart J. 152 (2006), 697-704). This is also confirmed by previous data from Jahns et al., presenting direct evidence that β1-AR auto-antibodies play a causal role in DCM and not merely correlate or are a consequence of myocardial tissue injury (Jahns, J. Clin. Invest. 113 (2004), 1419-1429).

In consequence, the removal of auto-antibodies directed against the second extracellular loop of the human β1-AR would be expected to lead to an improved clinical status in DCM patients. Initial clinical trials with patients suffering from DCM showed that not only cardiac auto-antibody titers were reduced, but also left ventricular function was improved after treatment with IgG immunadsorption (IA) (Felix., Am Coll Cardiol 35 (2000), 1590-1598; Wallukat, N. Engl. J. Med. 347 (2002), 180; Müller, N Engl J Med 347 (2002), 1806 and Müller, Circulation 101 (2000), 385-391). Another approach to lower cardiac auto-antibodies is the use of a peptide-based vaccine to reach antigen-specific tolerance and to reduce the response of an overactive immune system. Several (cyclo-) peptides homologous to the second extracellular loop of β1-AR are disclosed, e.g., in WO 01/21660 and proposes to apply these peptides for medical intervention of dilative cardiomyopathy (DCM). Moreover, it is mentioned, e.g., WO 01/21660 that these peptides may be modified in order to protect them against serum proteases, for example, by cyclization.

Both treatment strategies share the need of a reliable diagnostic assay for screening for auto-anti-β1-AR antibodies and thus reliably identify positive heart failure patients, preferably suffering from DCM. In the past, large efforts were undertaken to develop such an assay. These approaches can be divided into two classes:
assays which investigate the functional capability of the antibodies to activate the human β1 adrenoreceptor (β1-AR)
assays which analyse the binding characteristics of auto-antibodies that are directed against the second extracellular loop of the human β1 adrenoreceptor (ECII loop of the human β1-AR).

Functional assays, i.e. contractility effects on neonatal rat cardiomyocytes or chick embryos and receptor-mediated signalling cAMP levels, were established and adapted to detect functional anti-β1-AR antibodies (Nikolaev, Am. Coll. Cardiol. 50 (2007), 423-443; Wallukat, Mol Cell Cardiol. 27 (1995), 397-406, Erratum in: J Mol Cell Cardiol 27 (1995), 2529; Baba, Ther Apher Dial. 12 (2008), 109-116; Tutor, Cardiovasc Res 76 (2007), 51-60). All these functional assays are characterized by procedures which are time and cost consuming and which cannot reasonably be used to screen larger patient populations (n>1000) rapidly.

Binding of human auto-anti-β1-AR-antibodies was also investigated by using peptide-based ELISAs. To this end, a 26-meric peptide (His-Trp-Trp-Arg-Ala-Glu-Ser-Asp-Glu-Ala-Arg-Arg-Cys-Tyr-Asn-Asp-Pro-Lys-Cys-Cys-Asp-Phe-Val-Thr-Asn-Arg (SEQ ID NO:17)), which corresponds to the second extracellular loop (amino acid position 197-222) of the human β1-AR, was immobilized onto microtiter plates (Magnusson, J Clin Invest 86 (1990), 1658-1663 and Labovsky, Clin Exp Immunol 148 (2007), 440-449. This kind of assay is fully HTS (high throughput screening) adapted, but its use as a screening assay with diagnostic relevance had not yet been investigated in a larger population of patients and healthy controls simultaneously.

In the present study, patients suffering from heart failure, particularly DCM, were examined for the presence of auto-antibodies against the human β1-AR using a binding assay, particularly a cell based competitive ELISA assay, with either fully native human β1-AR or an assay using the human β1-AR ECII corresponding peptide (referring to the above mentioned 26-meric peptide His-Trp-Trp-Arg-Ala-Glu-Ser-Asp-Glu-Ala-Arg-Arg-Cys-Tyr-Asn-Asp-Pro-Lys-Cys-Cys-Asp-Phe-Val-Thr-Asn-Arg (SEQ ID NO:17) as respective binding targets.

In view of the present art, the technical problem underlying the present invention is the provision of improved means and methods for the diagnosis and prediction of a disease associated with human β1-adrenoreceptor (β1-AR).

The technical problem is solved by provision of the embodiments characterized in the claims.

The invention relates to antibodies/binding compounds that bind to the second extracellular loop of the human β1-adrenoreceptor (β1-AR). The antibodies of the present invention bind to the second extracellular loop of the human β1-adrenoreceptor that is or comprises the amino acid sequence as depicted in SEQ ID NO:17. The antibodies/binding compounds are obtainable from a host cell, e.g. a hybridoma, with a deposit number selected from the group consisting of DSM ACC3121, DSM ACC3174, DSM ACC3175, DSM ACC3176 and DSM ACC3177. These are the particular preferred binding compounds/antibodies of this invention. These binding compounds/antibodies are employed in the means and methods like diagnostic methods provided herein.

The present invention also relates to the establishment of a cell-based competitive ELISA for the detection of functionally active human anti-β1-AR auto-antibodies as above described. This assay uses the fully native β1-AR protein as target antigen to provide a correct folding of the extracellular domains which is a basic requirement to identify epitope-specific auto-antibodies. In order to optimize the specificity of the assay, a competitive approach was developed using the antibodies/binding compounds that bind to the second extracellular loop of the human β1-AR and are able to stimulate receptor activity.

Functionally relevant human anti-β1-AR auto-antibodies from patient sera are characterized by their capacity to bind to the same or overlapping epitopes and displace the test binding molecule/antibody and therefore reduce the immunological or biological signal like an ELISA signal. An epitope search by alanine permutation scanning has yielded hints that within the EC II loop of the β1-AR, the amino acid sequence NDPK (Asn-Asp-Pro-Lys) should be part of the relevant epitope.

Accordingly, the present invention relates to antibodies that bind to the second extracellular loop of the human β1-adrenoreceptor (β1-AR-ECII) having one or more desirable properties, including a high binding affinity. The antibodies described herein and in the diagnostic methods bind to the second extracellular loop of the human 11-adrenoreceptor (β1-AR-ECII), wherein said second extracellular loop of the human β1-adrenoreceptor β1-AR-ECII) is or comprises the amino acid sequence as depicted in SEQ ID NO:17. The anti-β1-AR-ECII antibodies described herein are produced by/obtainable from a host cell, for example a hybridoma, with a deposit number selected from the group consisting of DSM ACC3121, DSM ACC3174, DSM ACC3176 and DSM ACC3177. The invention also relates to the use of the antibodies of the present invention in a method for identifying patients having or being at risk of developing a disease associated with human β1-adrenoreceptor. As has been surprisingly found in the present invention, a binding compound/antibody or a derivative of said binding compound/antibody that is produced by/obtainable from the hybridoma cell line 23-6-7 with a deposit number of DSM ACC3121 (deposited by the Corimmun GmbH on Mar. 15, 2011 under the identification reference “b1ECII E3, 23-6-7 (anti-beta1-AR)”) exhibits increased affinity to the β1-adrenoreceptor compared to polyclonal (control) antibodies. Furthermore, the invention relates to (i) the mouse monoclonal antibodies or derivatives of said antibodies that are produced by/obtainable from the hybridoma cell lines 28-2-7 (deposited by the Corimmun GmbH on May 16, 2012 under the identification reference “b1ECII, 28-2-7” and the deposition number DSM ACC3175), 47-12-9 (as deposited by the Corimmun GmbH on May 16, 2012 under the identification reference “b1ECII, 47-12-9” and the deposition number DSM ACC3176), 50-1-5 (deposited by the Corimmun GmbH on May 16, 2012 under the identification reference “b1ECII, 50-1-5” and deposition number DSM ACC3177) and 55-4-10 and (ii) the rat monoclonal antibody 13F6 (deposited by the Corimmun GmbH on May 16, 2012 under the identification reference “13/F6” and the deposit number DSM ACC3174) or (iii) goat polyclonal antibodies (see FIGS. 3 to 5 of the appended Examples). It has also been shown and illustrated in the appended examples that the rat monoclonal antibody 13F6 is also characterized by a significantly increased affinity to the β1-adrenoreceptor (β1-AR) compared to the goat polyclonal (control) antibodies (FIG. 5). In view of this increased affinity to the β1-AR the monoclonal rat 13F6 antibody (that is obtainable from the host cell, for example a hybridoma, as deposited under DSM ACC3174) and derivatives of 13F6 can also be used, for example, as a positive control (PC) in a diagnostic method for identifying a patient having or being at risk of developing a disease associated with human β₁-adrenoreceptor as described herein below.

The term “β1-adrenoreceptor (β1-AR)” as used herein refers preferably to a human β1-adrenoreceptor, which is generally known to the skilled person. For example, the coding sequence can be obtained of the human β1-adrenergic receptor from a database known to the skilled person. For example, as used herein, the sequence (SEQ ID NOs:1 and 2) of the human β1-AR (also known as human (31 adrenoreceptor (ADRB1)) can be obtained from the database entry NM_{—}000684 (version NM_{—}000684.2; GI:110349783) and/or NP_{—}000675 (version number NP_{—}000675.1; GI:4557265).

In the context of the present invention, the nucleic acid sequence of the human β1-adrenoreceptor comprises the following (cDNA) sequence (referring to SEQ ID NO:1):

atgggcgcgg gggtgctcgt cctgggcgcc tccgagcccg
gtaacctgtc gtcggccgca ccgctccccg acggcgcggc
caccgcggcg cggctgctgg tgcccgcgtc gccgcccgcc
tcgttgctgc ctcccgccag cgaaagcccc gagccgctgt
ctcagcagtg gacagcgggc atgggtctgc tgatggcgct
catcgtgctg ctcatcgtgg cgggcaatgt gctggtgatc
gtggccatcg ccaagacgcc gcggctgcag acgctcacca
acctcttcat catgtccctg gccagcgccg acctggtcat
ggggctgctg gtggtgccgt tcggggccac catcgtggtg
tggggccgct gggagtacgg ctccttcttc tgcgagctgt
ggacctcagt ggacgtgctg tgcgtgacgg ccagcatcga
gaccctgtgt gtcattgccc tggaccgcta cctcgccatc
acctcgccct tccgctacca gagcctgctg acgcgcgcgc
gggcgcgggg cctcgtgtgc accgtgtggg ccatctcggc
cctggtgtcc ttcctgccca tcctcatgca ctggtggcgg
gcggagagcg acgaggcgcg ccgctgctac aacgacccca
agtgctgcga cttcgtcacc aaccgggcct acgccatcgc
ctcgtccgta gtctccttct acgtgcccct gtgcatcatg
gccttcgtgt acctgcgggt gttccgcgag gcccagaagc
aggtgaagaa gatcgacagc tgcgagcgcc gtttcctcgg
cggcccagcg cggccgccct cgccctcgcc ctcgcccgtc
cccgcgcccg cgccgccgcc cggacccccg cgccccgccg
ccgccgccgc caccgccccg ctggccaacg ggcgtgcggg
taagcggcgg ccctcgcgcc tcgtggccct gcgcgagcag
aaggcgctca agacgctggg catcatcatg ggcgtcttca
cgctctgctg gctgcccttc ttcctggcca acgtggtgaa
ggccttccac cgcgagctgg tgcccgaccg cctcttcgtc
ttcttcaact ggctgggcta cgccaactcg gccttcaacc
ccatcatcta ctgccgcagc cccgacttcc gcaaggcctt
ccagggactg ctctgctgcg cgcgcagggc tgcccgccgg
cgccacgcga cccacggaga ccggccgcgc gcctcgggct
gtctggcccg gcccggaccc ccgccatcgc ccggggccgc
ctcggacgac gacgacgacg atgtcgtcgg ggccacgccg
cccgcgcgcc tgctggagcc ctgggccggc tgcaacggcg
gggcggcggc ggacagcgac tcgagcctgg acgagccgtg
ccgccccggc ttcgcctcgg aatccaaggt gtag

The amino acid sequence of the human β1-adrenoreceptor is shown in SEQ ID NO:2):

Met Gly Ala Gly Val Leu Val Leu Gly Ala Ser Glu
Pro Gly Asn Leu Ser Ser Ala Ala Pro Leu Pro Asp
Gly Ala Ala Thr Ala Ala Arg Leu Leu Val Pro Ala
Ser Pro Pro Ala Ser Leu Leu Pro Pro Ala Ser Glu
Ser Pro Glu Pro Leu Ser Gln Gln Trp Thr Ala Gly
Met Gly Leu Leu Met Ala Leu Ile Val Leu Leu Ile
Val Ala Gly Asn Val Leu Val Ile Val Ala Ile Ala
Lys Thr Pro Arg Leu Gln Thr Leu Thr Asn Leu Phe
Ile Met Ser Leu Ala Ser Ala Asp Leu Val Met Gly
Leu Leu Val Val Pro Phe Gly Ala Thr Ile Val Val
Trp Gly Arg Trp Glu Tyr Gly Ser Phe Phe Cys Glu
Leu Trp Thr Ser Val Asp Val Leu Cys Val Thr Ala
Ser Ile Glu Thr Leu Cys Val Ile Ala Leu Asp Arg
Tyr Leu Ala Ile Thr Ser Pro Phe Arg Tyr Gln Ser
Leu Leu Thr Arg Ala Arg Ala Arg Gly Leu Val Cys
Thr Val Trp Ala Ile Ser Ala Leu Val Ser Phe Leu
Pro Ile Leu Met His Trp Trp Arg Ala Glu Ser Asp
Glu Ala Arg Arg Cys Tyr Asn Asp Pro Lys Cys Cys
Asp Phe Val Thr Asn Arg Ala Tyr Ala Ile Ala Ser
Ser Val Val Ser Phe Tyr Val Pro Leu Cys Ile Met
Ala Phe Val Tyr Leu Arg Val Phe Arg Glu Ala Gln
Lys Gln Val Lys Lys Ile Asp Ser Cys Glu Arg Arg
Phe Leu Gly Gly Pro Ala Arg Pro Pro Ser Pro Ser
Pro Ser Pro Val Pro Ala Pro Ala Pro Pro Pro Gly
Pro Pro Arg Pro Ala Ala Ala Ala Ala Thr Ala Pro
Leu Ala Asn Gly Arg Ala Gly Lys Arg Arg Pro Ser
Arg Leu Val Ala Leu Arg Glu Gln Lys Ala Leu Lys
Thr Leu Gly Ile Ile Met Gly Val Phe Thr Leu Cys
Trp Leu Pro Phe Phe Leu Ala Asn Val Val Lys Ala
Phe His Arg Glu Leu Val Pro Asp Arg Leu Phe Val
Phe Phe Asn Trp Leu Gly Tyr Ala Asn Ser Ala Phe
Asn Pro Ile Ile Tyr Cys Arg Ser Pro Asp Phe Arg
Lys Ala Phe Gln Gly Leu Leu Cys Cys Ala Arg Arg
Ala Ala Arg Arg Arg His Ala Thr His Gly Asp Arg
Pro Arg Ala Ser Gly Cys Leu Ala Arg Pro Gly Pro
Pro Pro Ser Pro Gly Ala Ala Ser Asp Asp Asp Asp
Asp Asp Val Val Gly Ala Thr Pro Pro Ala Arg Leu
Leu Glu Pro Trp Ala Gly Cys Asn Gly Gly Ala Ala
Ala Asp Ser Asp Ser Ser Leu Asp Glu Pro Cys Arg
Pro Gly Phe Ala Ser Glu Ser Lys Val

The human β1-adrenoreceptor refers to a receptor having seven transmenbrane regions within the amino acid positions 59-83, 96-120, 133-152, 177-196, 223-243, 327-346 and 359-378 of the amino acid sequence as depicted in SEQ ID NO:2. The second extracellular loop region of the human β1-adrenoreceptor lies within the amino acid positions 197-222 of the amino acid sequence as depicted in SEQ ID NO:2 (referring to the amino acid sequence His-Trp-Trp-Arg-Ala-Glu-Ser-Asp-Glu-Ala-Arg-Arg-Cys-Tyr-Asn-Asp-Pro-Lys-Cys-Cys-Asp-Phe-Val-Thr-Asn-Arg; SEQ ID NO:17).

Furthermore, as is detailed and exemplified in the appended examples, the antibody or a derivative thereof of the present invention that is produced by/obtainable from the host cell, for example a hybridoma, with a deposit number DSM ACC3121 (referring to the host cell, for example a hybridoma, with the identification reference “b1ECII E3, 23-6-7 (anti-beta1-AR)”) can be used in a method for identifying patient having or being at risk of developing a idiopathic dilated cardiomyopathy (DCM) as it can be detected by the identification of the auto-antibodies which are directed against the human β1-adrenoreceptor (β1-AR-ECII). The invention also relates to the antibody or a derivative thereof that is produced by/obtainable from the host cell, for example a hybridoma, with the deposit number DSM ACC3121 (referring to the hybridoma cell line with the identification reference “b1ECII E3, 23-6-7 (anti-beta1-AR)”) and its use in a method for identifying patient having or being at risk of developing a disease associated with human β1-adrenoreceptor. The invention also relates to the antibody or a derivative thereof that is produced by/obtainable from the host cell, for example a hybridoma with the deposit number DSM ACC3175 (referring to the hybridoma cell line 28-2-7 with the identification reference “b1ECII, 28-2-7”) and its use in a method for identifying patient having or being at risk of developing a disease associated with human β1-adrenoreceptor. The invention also relates to the antibody or a derivative thereof that is produced by/obtainable from the host cell, for example a hybridoma, with the deposit number DSM ACC3176 (referring to the hybridoma cell line 47-12-9 with the identification reference “b1ECII, 47-12-9”) and its use in a method for identifying patient having or being at risk of developing a disease associated with human β1-adrenoreceptor. The invention also relates to the antibody or a derivative thereof that is produced by/obtainable from the host cell, for example a hybridoma, with the deposit number DSM ACC3177 (referring to the hybridoma cell line 50-1-5 with the identification reference “b1ECII, 50-1-5”) and its use in a method for identifying patient having or being at risk of developing a disease associated with human β1-adrenoreceptor. The invention also relates to the antibody or a derivative thereof that is produced by/obtainable from the host cell, for example a hybridoma, with the deposit number DSM ACC3174 (referring to the hybridoma cell line with the identification reference “13/F6”) and its use in a method for identifying patient having or being at risk of developing a disease associated with human β1-adrenoreceptor.

In the context of the disclosed and the descriptive terms, it is to be understood that the term “produced by” and “obtainable from” does not relate to the specific monoclonal antibodies but also to derivatives and variants of said deposited antibodies. Such derivatives and variants have at least parts of the CDR sequences of the deposited monoclonal antibodies. Derivatives and variants comprise but are not limited to CDR grafted, humanized antibodies, Fab, Fab′, Fab′-SH, FV, scFV, F(ab′)2, and a diabody.

As used herein the term “antibody fragment” or “binding fragment” of an antibody/binding molecule (the parental antibody/binding molecule) encompasses a fragment or derivative of an antibody/binding molecule, typically including at least a portion of the antigen binding or variable regions (e.g., one or more CDRs) of the parental antibodies, that retains at least some of the binding specificity of the parental antibody. Particularly the parental antibody/binding molecule refers herein to the antibodies that bind to the second extracellular loop of the human β1-adrenoreceptor (β1-AR) that are produced by/obtainable from a host cell, for example a hybridoma, with a deposit number selected from the group consisting of DSM ACC3121, DSM ACC3174, DSM ACC3176 and DSM ACC3177. Examples of antibody binding fragments include, but are not limited to, Fab, Fab′, F(ab′)₂, and Fv fragments; diabodies; linear antibodies; single-chain antibody molecules, e.g., sc-Fv; and multispecific antibodies formed from antibody fragments. Typically, a binding fragment or derivative retains at least 10% of the binding activity to the second extracellular of the human β1-adrenoreceptor (β1-AR) when that activity is expressed on a molar basis. Preferably, a binding fragment or derivative retains at least 20%, 50%, 70%, 80%, 90%, 95% or 100% or more of the binding affinity binding activity to the second extracellular of the human β1-adrenoreceptor (β1-AR) as the parental antibody, particularly the deposited monoclonal antibodies. It is also intended that an binding fragment that binds to the to the second extracellular of the human β1-adrenoreceptor (β1-AR) can include conservative amino acid substitutions (referred to as “conservative variants” of the antibody) that do not substantially alter its biologic activity.

Generally, the binding compound of the present invention is an antibody which binds against the second extracellular loop of the human β1-adrenoreceptor (β1-AR-ECII). Particularly, the binding compound of the present invention is an antibody that binds against the second extracellular loop of the human β1-adrenoreceptor (β1-AR-ECII) which comprises or consists of VH domain (heavy chain variable region) and VL domain (light chain variable region) with at least 95%, 90%, 85%, 75%, 70%, 65%, 60%, 55% or 50% sequence homology with the sequences of SEQ ID NO:4 and SEQ ID NO:6 (or SEQ ID NO:3 and SEQ ID NO:5 if reference to the corresponding nucleic acid sequences of the heavy and light chain variable region is made). Furthermore, the binding compound of the present invention is an antibody that comprises VH and VL domains having up to 0, 1, 2, 3, 4, 5, 6, 7, 8, 9, 10 or more conservative amino acid substitutions with reference to the sequences of SEQ ID NO:4 and SEQ ID NO:6. Moreover, the binding compound of the present invention is an antibody or binding fragment thereof, e.g., an antibody fragment selected from the group consisting of Fab, Fab′, Fab′-SH, FV, scFV, F(ab′)2, and a diabody.

The invention also relates to an antibody that binds against the second extracellular loop of the human β1-adrenoreceptor (β1-AR-ECII) which comprises or consists of VH domain (heavy chain variable region) and VL domain (light chain variable region) with at least 95%, 90%, 85%, 75%, 70%, 65%, 60%, 55% or 50% sequence homology with the sequences of SEQ ID NO:33 and SEQ ID NO:31 (or SEQ ID NO:32 and SEQ ID NO:30 if reference to the corresponding nucleic acid sequences of the heavy and light chain variable region is made). Furthermore, the binding compound of the present invention is an antibody that comprises VH and VL domains having up to 0, 1, 2, 3, 4, 5, 6, 7, 8, 9, 10 or more conservative amino acid substitutions with reference to the sequences of SEQ ID NO:33 and SEQ ID NO:31. Moreover, the binding compound of the present invention is an antibody or binding fragment thereof, e.g., an antibody fragment selected from the group consisting of Fab, Fab′, Fab′-SH, FV, scFV, F(ab′)2, and a diabody.

The invention also relates to an antibody that binds against the second extracellular loop of the human β1-adrenoreceptor (β1-AR-ECII) which comprises or consists of VH domain (heavy chain variable region) and VL domain (light chain variable region) with at least 95%, 90%, 85%, 75%, 70%, 65%, 60%, 55% or 50% sequence homology with the sequences of SEQ ID NO:43 and SEQ ID NO:41 (or SEQ ID NO:42 and SEQ ID NO:40 if reference to the corresponding nucleic acid sequences of the heavy and light chain variable region is made). Furthermore, the binding compound of the present invention is an antibody that comprises VH and VL domains having up to 0, 1, 2, 3, 4, 5, 6, 7, 8, 9, 10 or more conservative amino acid substitutions with reference to the sequences of SEQ ID NO:43 and SEQ ID NO:41. Moreover, the binding compound of the present invention is an antibody or binding fragment thereof, e.g., an antibody fragment selected from the group consisting of Fab, Fab′, Fab′-SH, FV, scFV, F(ab′)2, and a diabody.

The invention also relates to an antibody that binds against the second extracellular loop of the human β1-adrenoreceptor (β1-AR-ECII) which comprises or consists of VH domain (heavy chain variable region) and VL domain (light chain variable region) with at least 95%, 90%, 85%, 75%, 70%, 65%, 60%, 55% or 50% sequence homology with the sequences of SEQ ID NO:53 and SEQ ID NO:51 (or SEQ ID NO:52 and SEQ ID NO:50 if reference to the corresponding nucleic acid sequences of the heavy and light chain variable region is made). Furthermore, the binding compound of the present invention is an antibody that comprises VH and VL domains having up to 0, 1, 2, 3, 4, 5, 6, 7, 8, 9, 10 or more conservative amino acid substitutions with reference to the sequences of SEQ ID NO:53 and SEQ ID NO:51. Moreover, the binding compound of the present invention is an antibody or binding fragment thereof, e.g., an antibody fragment selected from the group consisting of Fab, Fab′, Fab′-SH, FV, scFV, F(ab′)2, and a diabody.

The invention also relates to an antibody that binds against the second extracellular loop of the human β1-adrenoreceptor (β1-AR-ECII) which comprises or consists of VH domain (heavy chain variable region) and VL domain (light chain variable region) with at least 95%, 90%, 85%, 75%, 70%, 65%, 60%, 55% or 50% sequence homology with the sequences of SEQ ID NO:63 and SEQ ID NO:61 (or SEQ ID NO:62 and SEQ ID NO:60 if reference to the corresponding nucleic acid sequences of the heavy and light chain variable region is made). Furthermore, the binding compound of the present invention is an antibody that comprises VH and VL domains having up to 0, 1, 2, 3, 4, 5, 6, 7, 8, 9, 10 or more conservative amino acid substitutions with reference to the sequences of SEQ ID NO:63 and SEQ ID NO:61. Moreover, the binding compound of the present invention is an antibody or binding fragment thereof, e.g., an antibody fragment selected from the group consisting of Fab, Fab′, Fab′-SH, FV, scFV, F(ab′)2, and a diabody.

In the context of the present invention, the antibody as described herein is a full antibody (immunoglobulin, like an IgG1, an IgG2, an IgG2b, an IgG3, an IgG4, an IgA, an IgM, an IgD or an IgE), an F(ab)-, Fabc-, Fv-, Fab′-, F(ab′)₂-fragment, a single-chain antibody, a chimeric antibody, a CDR-grafted antibody, a bivalent antibody-construct, an antibody-fusion protein or a synthetic antibody.

Furthermore, the scope of the present invention comprises any binding compound comprising one or more complementarity determining regions (CDRs) (3 light chain CDRs and/or 3 heavy chain CDRs) and/or framework regions of any of the light chain immunoglobulin or heavy chain immunoglobulins as identified by the methods identified in Chothia, J. Mol. Biol. 186 (1985), 651-663; Novotny and Haber, Proc. Natl. Acad. Sci. USA 82 (1985), 4592-4596 or Kabat, Sequences of Proteins of Immunological Interest, National Institutes of Health, Bethesda, Md., (1987)).

The present invention relates to antibodies that bind to the second extracellular loop of the human β1-adrenoreceptor comprising one or more complementarity determining regions (CDRs) as shown in the following. The antibody refers to a mouse monoclonal binding compound/antibody or a derivative thereof that is produced by/obtainable from the hydridoma deposited under the deposit number (accession number) DSM ACC3121 comprising the following CDRs of the light chain variable region (VL domain) or the heavy chain variable region (VH domain), respectively. Accordingly, the antibody that is obtainable from the host cell, for example a hybridoma, with the deposit number DSM ACC3121 of the present invention comprises one or more complementarity determining regions (CDRs) (according to the classification system of Kabat) selected from the group consisting of:

CDRH1:
(SEQ ID NO: 7)
Asp-Tyr-Tyr-Met-His
CDRH2:
(SEQ ID NO: 8)
Arg-Ile-Asn-Pro-Tyr-Ser-Gly-Ala-Pro-Ser-Tyr-
Thr-Gln-Asn-Phe-Lys-Ala
CDRH3:
(SEQ ID NO: 9)
Ala-Asn-Trp-Asp-Gly-Tyr-Phe-Asp-Tyr
CDRL1:
(SEQ ID NO: 10)
Ser-Ala-Ser-Ser-Ser-Val-Ser-Tyr-Met-Tyr
CDRL2:
(SEQ ID NO: 11)
Asp-Thr-Ser-Lys-Leu-Ala-Ser
CDRL3:
(SEQ ID NO: 12)
Gln-Gln-Trp-Ser-Ser-Asn-Pro-Trp-Thr

The invention also relates to the antibody or a derivative thereof that is obtainable from the deposit number DSM ACC3174 and that binds to the second extracellular loop of the human β1-adrenoreceptor comprising one or more complementarity determining regions (CDRs) as shown in the following. The antibody refers to a rat monoclonal binding compound/binding or a derivative thereof comprising the following CDRs of the light chain variable region (VL domain) or the heavy chain variable region (VH domain), respectively. Accordingly, the antibody or derivative thereof that is obtainable from the host cell, for example a hybridoma, with the deposit number DSM ACC3174 of the present invention comprises one or more complementarity determining regions (CDRs) (according to the classification system of Kabat) selected from the group consisting of CDRL1 as depicted in SEQ ID NO:34; CDRL2 as depicted in SEQ ID NO:35; CDRL3 as depicted in SEQ ID NO:36; CDRH1 as depicted in SEQ ID NO: 37; CDRH2 as depicted in SEQ ID NO:38 and CDRH3 as depicted in SEQ ID NO:39.

The invention also relates to the antibody or a derivative thereof that is obtainable from the deposit number DSM ACC3175 and that binds to the second extracellular loop of the human β1-adrenoreceptor comprising one or more complementarity determining regions (CDRs) as shown in the following. The antibody refers to a mouse monoclonal binding compound/antibody comprising the following CDRs of the light chain variable region (VL domain) or the heavy chain variable region (VH domain), respectively. Accordingly, the antibody or derivative thereof that is obtainable from the host cell, for example a hybridoma, with the deposit number DSM ACC3175 of the present invention comprises one or more complementarity determining regions (CDRs) (according to the classification system of Kabat) selected from the group consisting of CDRL1 as depicted in SEQ ID NO:44; CDRL2 as depicted in SEQ ID NO:45; CDRL3 as depicted in SEQ ID NO:46; CDRH1 as depicted in SEQ ID NO:47; CDRH2 as depicted in SEQ ID NO:48 and CDRH3 as depicted in SEQ ID NO:49.

The invention also relates to the antibody or a derivative thereof that is obtainable from the deposit number DSM ACC3176 and that binds to the second extracellular loop of the human β1-adrenoreceptor comprising one or more complementarity determining regions (CDRs) as shown in the following. The antibody refers to a mouse monoclonal binding compound (antibody) comprising the following CDRs of the light chain variable region (VL domain) or the heavy chain variable region (VH domain), respectively. Accordingly, the antibody or a derivative thereof that is obtainable from the host cell, for example a hybridoma, with the deposit number DSM ACC3176 of the present invention comprises one or more complementarity determining regions (CDRs) (according to the classification system of Kabat) selected from the group consisting of CDRL1 as depicted in SEQ ID NO:54; CDRL2 as depicted in SEQ ID NO:55; CDRL3 as depicted in SEQ ID NO:56; CDRH1 as depicted in SEQ ID NO:57; CDRH2 as depicted in SEQ ID NO:58 and CDRH3 as depicted in SEQ ID NO:59.

The invention also relates to the antibody or a derivative thereof that is obtainable from the deposit number DSM ACC3177 and that binds to the second extracellular loop of the human β1-adrenoreceptor comprising one or more complementarity determining regions (CDRs) as shown in the following. The antibody refers to a mouse monoclonal binding compound (antibody) comprising the following CDRs of the light chain variable region (VL domain) or the heavy chain variable region (VH domain), respectively. Accordingly, the antibody or a derivative thereof that is obtainable from the host cell (hybridoma) with the deposit number DSM ACC3177 of the present invention comprises one or more complementarity determining regions (CDRs) (according to the classification system of Kabat) selected from the group consisting of CDRL1 as depicted in SEQ ID NO:64; CDRL2 as depicted in SEQ ID NO:65; CDRL3 as depicted in SEQ ID NO:66; CDRH1 as depicted in SEQ ID NO:67; CDRH2 as depicted in SEQ ID NO:68 and CDRH3 as depicted in SEQ ID NO:69.

Furthermore, the binding compounds of the present invention refer to the mouse monoclonal binding compound (antibody) that is produced by (obtainable from) the hydridoma (host cell) with the deposit number DSM ACC3121 comprising or consisting of a heavy chain variable region (VH domain) and/or a light chain variable region (VL domain) as shown in the following.

Accordingly, the binding compound/antibody that is produced by/obtainable from the host cell, for example a hydridoma, with the deposit number DSM ACC3121 comprises the following cDNA sequences or the deduced amino acid sequences: the cDNA-sequence of the variable region of the heavy chain of SEQ ID NO:3;

CTGGTGAAGC CTGGGGCTTC AGTGAAGATA TCCTGCAAGG
CTTCTGGTTA CTCATTCACT GACTACTACA TGCACTGGGT
GAAGCAAAGC CATGTAAAGA GCCTTGAGTG GATTGGACGT
ATTAATCCTT ACAGTGGTGC TCCTAGCTAC ACCCAGAATT
TCAAGGCCAA GGCCAGCTTG ACTGTAGATA AGTCCTCCAG
CACAGCCTAC ATGGAGCTCC ACAGCCTGAC ATCTGAGGAC
TCTGCAGTCT ATTACTGCGC AAGAGCTAAC TGGGACGGGT
ACTTTGACTA CTGGGGCCAA GGCACCACTC TCACA

the amino acid sequence of the variable region of the heavy chain of SEQ ID NO:4;

Leu Val Lys Pro Gly Ala Ser Val Lys Ile Cys Lys
Ala Ser Gly Tyr Ser Phe Thr Asp Tyr Tyr Met His
Trp Val Lys Gln Ser His Val Lys Ser Leu Glu Trp
Ile Gly Arg Ile Asn Pro Tyr Ser Gly Ala Pro Ser
Tyr Thr Gln Asn Phe Lys Ala Lys Ala Ser Leu Thr
Val Asp Lys Ser Ser Ser Thr Ala Tyr Met Glu Leu
His Ser Leu Thr Ser Glu Asp Ser Ala Val Tyr Tyr
Cys Ala Arg Ala Asn Trp Asp Gly Tyr Phe Asp Tyr
Trp Gly Gln Gly Thr Thr Leu Thr

the cDNA-sequence of the variable region of the light chain of SEQ ID NO:5;

ATGTCTGCAT CTCCAGGGGA GAAGGTCACC ATGACCTGCA
GTGCCAGCTC AAGTGTAAGT TACATGTACT GGTACCAGCA
GAAGTCAGGC ACCTCCCCCA AAAGATGGAT TTATGACACA
TCCAAATTGG CTTCTGGAGT CCCTGTTCGC TTCAGTGGCA
GTGGGTCTGG GACCTCTTAC TCTCTCACAA TCAGCAGCAT
GGAGGCTGAA GATGCTGCCA CTTATTACTG CCAGCAGTGG
AGTAGTAACC CATGGACGTT CGGTGGAGGC ACCAAATTGG
AAATCAAACG G

and the amino acid sequence of the variable region of the light chain of SEQ ID NO:6.

Met Ser Ala Ser Pro Gly Glu Lys Val Thr Met Thr
Cys Ser Ala Ser Ser Ser Val Ser Tyr Met Tyr Trp
Tyr Gln Gln Lys Ser Gly Thr Ser Pro Lys Arg Trp
Ile Tyr Asp Thr Ser Lys Leu Ala Ser Gly Val Pro
Val Arg Phe Ser Gly Ser Gly Ser Gly Thr Ser Tyr
Ser Leu Thr Ile Ser Ser Met Glu Ala Glu Asp Ala
Ala Thr Tyr Tyr Cys Gln Gln Trp Ser Ser Asn Pro
Trp Thr Phe Gly Gly Gly Thr Lys Leu Glu Ile Lys
Arg

The invention also relates to the rat monoclonal binding compound/antibody that is produced by/obtainable from the host cell, for example a hybridoma, with the deposit number DSM ACC3174 comprising or consisting of a heavy chain variable region (VH domain) and/or a light chain variable region (VL domain) as shown in SEQ ID NO:33 (VH domain) and/or SEQ ID NO:31 (VL domain).

Accordingly, the antibody that is produced by/obtainable from the host cell, for example a hydridoma, with the deposit number DSM ACC3174 comprises the cDNA-sequences or the deduced amino acid sequence as shown in SEQ ID NO:30 (cDNA-sequence of the variable region of the light chain); SEQ ID NO:31 ((deduced) amino acid sequence of the variable region of the light chain); SEQ ID NO:32 (cDNA-sequence of the variable region of the heavy chain) and SEQ ID NO:33 ((deduced) amino acid sequence of the variable region of the heavy chain).

The invention also relates to the rat monoclonal binding compound/antibody that is produced by/obtainable from the host cell, for example a hybridoma with the deposit number DSM ACC3175 comprising or consisting of a heavy chain variable region (VH domain) and/or a light chain variable region (VL domain) as shown in SEQ ID NO:43 (VH domain) and/or SEQ ID NO:41 (VL domain).

Accordingly, the antibody that is produced by/obtainable from the host cell, for example a hydridoma, with the deposit number DSM ACC3175 comprises the cDNA-sequences or the deduced amino acid sequence as shown in SEQ ID NO:40 (cDNA-sequence of the variable region of the light chain); SEQ ID NO:41 ((deduced) amino acid sequence of the variable region of the light chain); SEQ ID NO:42 (cDNA-sequence of the variable region of the heavy chain) and SEQ ID NO:43 ((deduced) amino acid sequence of the variable region of the heavy chain).

The invention also relates to the rat monoclonal binding compound/antibody that is produced by/obtainable from the host cell, for example a hybridoma with the deposit number DSM ACC3176 comprising or consisting of a heavy chain variable region (VH domain) and/or a light chain variable region (VL domain) as shown in SEQ ID NO:53 (VH domain) and/or SEQ ID NO:51 (VL domain).

Accordingly, the antibody that is produced by/obtainable from the host cell, for example a hydridoma, with the deposit number DSM ACC3176 comprises the cDNA-sequences or the deduced amino acid sequence as shown in SEQ ID NO:50 (cDNA-sequence of the variable region of the light chain); SEQ ID NO:51 ((deduced) amino acid sequence of the variable region of the light chain); SEQ ID NO:52 (cDNA-sequence of the variable region of the heavy chain) and SEQ ID NO:53 ((deduced) amino acid sequence of the variable region of the heavy chain).

The invention also relates to the rat monoclonal binding compound/antibody that is produced by/obtainable from the host cell, for example a hybridoma with the deposit number DSM ACC3177 comprising or consisting of a heavy chain variable region (VH domain) and/or a light chain variable region (VL domain) as shown in SEQ ID NO:63 (VH domain) and/or SEQ ID NO:61 (VL domain).

Accordingly, the antibody that is produced by/obtainable from the host cell, for example a hydridoma, with the deposit number DSM ACC3177 comprises the cDNA sequences or the deduced amino acid sequence as shown in SEQ ID NO:60 (cDNA-sequence of the variable region of the light chain); SEQ ID NO:61 ((deduced) amino acid sequence of the variable region of the light chain); SEQ ID NO:62 (cDNA-sequence of the variable region of the heavy chain) and SEQ ID NO:63 ((deduced) amino acid sequence of the variable region of the heavy chain).

The term “binding compound” refers to both antibodies and binding fragments thereof. Accordingly in the context of the present invention, the antibody is a chimeric, humanized, bispecific or fully-human antibody.

Accordingly, in the present invention, the binding compounds refer to (a) monoclonal or polyclonal antibodies (antibody), preferably to (a) (mouse/murine) monoclonal antibody/antibodies. The antibody that is obtainable from the host cell, for example a hybridoma, with the deposit number DSM ACC3121 is a (mouse/murine) monoclonal antibody. The antibody that is obtainable from the host cell, for example a hybridoma, with the deposit number DSM ACC3175 is a (mouse/murine) monoclonal antibody. The antibody that is obtainable from the host cell, for example a hybridoma, with the deposit number DSM ACC3176 is a (mouse/murine) monoclonal antibody. The antibody that is obtainable from the host cell, for example a hybridoma, with the deposit number DSM ACC3177 is a (mouse/murine) monoclonal antibody.

The invention also relates to the antibody that is obtainable from the host cell (hybridoma) with the deposit number DSM ACC3174, wherein said antibody is a (rat) monoclonal antibody.

The term “monoclonal antibody” as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Monoclonal antibodies are advantageous in that they may be synthesized by a hybridoma culture, essentially uncontaminated by other immunoglobulins. The modified “monoclonal” indicates the character of the antibody as being amongst a substantially homogeneous population of antibodies, and is not to be construed as requiring production of the antibody by any particular method. As mentioned above, the monoclonal antibodies to be used in accordance with the present invention may be made by the hybridoma method described by Kohler, Nature 256 (1975), 495.

The term “polyclonal antibody” as used herein, refers to an antibody which was produced among or in the presence of one or more other, non-identical antibodies. In general, polyclonal antibodies are produced from a B-lymphocyte in the presence of several other B-lymphocytes which produced non-identical antibodies. Usually, polyclonal antibodies are obtained directly from an immunized animal.

The term “bispecific” or “bifunctional antibody” as used herein refers to an artificial hybrid antibody having two different heavy/light chain pairs and two different binding sites. Bispecific antibodies can be produced by a variety of methods including fusion of hybridomas or linking of Fab′ fragments. See, e.g., Songsivilai, Clin. Exp. Immunol. 79 (1990), 315-321 and Kostelny, J Immunol. 148 (1992), 1547-1553. In addition, bispecific antibodies may be formed as “diabodies” (Holliger, Proc. Nat. Acad. Sci. USA 90 (1993), 6444-6448) or as “Janusins” (Traunecker, EMBO J. 10 (1991), 3655-3659 and Traunecker, Int. J. Cancer Suppl. 7 (1992), 51-52).

The term “fully-human antibody” as used herein refers to an antibody which comprises human immunoglobulin protein sequences only. A fully human antibody may contain murine carbohydrate chains if produced in a mouse, in a mouse cell or in a hybridoma derived from a mouse cell. Similarly, “mouse antibody” or “murine antibody” refers to an antibody which comprises mouse/murine immunoglobulin protein sequences only. Alternatively, a “fully-human antibody” may contain rat carbohydrate chains if produced in a rat, in a rat cell, in a hybridoma derived from a rat cell. Similarly, the term “rat antibody” refers to an antibody that comprises rat immunoglobulin sequences only. Fully-human antibodies may also be produced, for example, by phage display which is a widely used screening technology which enables production and screening of fully human antibodies. Also phage antibodies can be used in context of this invention. Phage display methods are described, for example, in U.S. Pat. No. 5,403,484, U.S. Pat. No. 5,969,108 and U.S. Pat. No. 5,885,793. Another technology which enables development of fully-human antibodies involves a modification of mouse hybridoma technology. Mice are made transgenic to contain the human immunoglobulin locus in exchange for their own mouse genes (see, for example, U.S. Pat. No. 5,877,397).

The term “chimeric antibodies”—in an embodiment of the invention, refers to an antibody which comprises a variable region of the present invention fused or chimerized with an antibody region (e.g., constant region) from another, human or non-human species (e.g., mouse, horse, rabbit, dog, cow, chicken).

The term antibody also relates to recombinant human antibodies, heterologous antibodies and heterohybrid antibodies. The term “recombinant human antibody” includes all human sequence antibodies that are prepared, expressed, created or isolated by recombinant means, such as antibodies isolated from an animal (e.g., a mouse) that is transgenic for human immunoglobulin genes; antibodies expressed using a recombinant expression vector transfected into a host cell, antibodies isolated from a recombinant, combinatorial human antibody library, or antibodies prepared, expressed, created or isolated by any other means that involves splicing of human immunoglobulin gene sequences to other DNA sequences. Such recombinant human antibodies have variable and constant regions (if present) derived from human germline immunoglobulin sequences. Such antibodies can, however, be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

A “heterologous antibody” is defined in relation to the transgenic non-human organism producing such an antibody. This term refers to an antibody having an amino acid sequence or an encoding nucleic acid sequence corresponding to that found in an organism not consisting of the transgenic non-human animal, and generally from a species other than that of the transgenic non-human animal.

The term “heterohybrid antibody” refers to an antibody having light and heavy chains of different organismal origins. For example, an antibody having a human heavy chain associated with a murine light chain is a heterohybrid antibody. Examples of heterohybrid antibodies include chimeric and humanized antibodies.

The term antibody also relates to humanized antibodies. “Humanized” forms of non-human (e.g. murine or rabbit) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Often, humanized antibodies are human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Furthermore, humanized antibody may comprise residues, which are found neither in the recipient antibody nor in the imported CDR or framework sequences. These modifications are made to further refine and optimize antibody performance. In general, the humanized antibody will comprise substantially all of at least one, and typically two variable domains, in which all or substantially all of the CDR regions correspond to those of a non-human immunoglobulin and all or substantially all of the FR regions are those of a human immunoglobulin consensus sequence. The humanized antibody may also comprise at least a portion of an immunoglobulin constant region (Fc), typically that of a human immunoglobulin. For further details, see: JonesNature 321 (1986), 522-525; Reichmann Nature 332 (1998), 323-327 and Presta Curr Op Struct Biol 2 (1992), 593-596.

A popular method for humanization of antibodies involves CDR grafting, where a functional antigen-binding site from a non-human ‘donor’ antibody is grafted onto a human ‘acceptor’ antibody. CDR grafting methods are known in the art and described, for example, in U.S. Pat. No. 5,225,539, U.S. Pat. No. 5,693,761 and U.S. Pat. No. 6,407,213. Another related method is the production of humanized antibodies from transgenic animals that are genetically engineered to contain one or more humanized immunoglobulin loci which are capable of undergoing gene rearrangement and gene conversion (see, for example, U.S. Pat. No. 7,129,084).

Accordingly, in context of the present invention, the term “antibody” or “binding compound” relates to full immunoglobulin molecules as well as to parts of such immunoglobulin molecules. Furthermore, the term relates, as discussed above, to modified and/or altered antibody molecules. The term also relates to recombinantly or synthetically generated/synthesized antibodies. The term also relates to intact antibodies as well as to antibody fragments thereof, like, separated light and heavy chains, Fab, Fv, Fab′, Fab′-SH, F(ab′)2. The term antibody also comprises but is not limited to fully-human antibodies, chimeric antibodies, humanized antibodies, CDR-grafted antibodies and antibody constructs, like single chain Fvs (scFv) or antibody-fusion proteins.

“Single-chain Fv” or “scFv” antibody fragments have, in the context of the invention, the V_{H} and V_{L} domains of an antibody, wherein these domains are present in a single polypeptide chain. Generally, the scFv polypeptide further comprises a polypeptide linker between the V_{H} and V_{L} domains which enables the scFv to form the desired structure for antigen binding. Techniques described for the production of single chain antibodies are described, e.g., in Plückthun in The Pharmacology of Monoclonal Antibodies, Rosenburg and Moore eds. Springer-Verlag, N.Y. (1994), 269-315.

A “Fab fragment” as used herein is comprised of one light chain and the C_{H}1 and variable regions of one heavy chain. The heavy chain of a Fab molecule cannot form a disulfide bond with another heavy chain molecule.

An “Fc” region contains two heavy chain fragments comprising the C_{H}2 and C_{H}3 domains of an antibody. The two heavy chain fragments are held together by two or more disulfide bonds and by hydrophobic interactions of the C_{H}3 domains.

A “Fab′ fragment” contains one light chain and a portion of one heavy chain that contains the V_{H} domain and the C_{H}1 domain and also the region between the C_{H}1 and C_{H}2 domains, such that an interchain disulfide bond can be formed between the two heavy chains of two Fab′ fragments to form a F(ab′)₂ molecule.

A “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains.

The “Fv region” comprises the variable regions from both the heavy and light chains, but lacks the constant regions.

In the context of the present invention, the binding compound may be also an antibody that is produced by/obtainable from the host cell, for example a hybridoma, with the deposit number DSM ACC3121. Furthermore, the binding molecule/antibody of the present invention comprises a heavy chain constant region, for example a mouse constant region, such as γ1, γ2a, γ2b or γ3 mouse heavy chain constant region or a variant thereof. The binding molecule/antibody of the present invention may also comprise a light chain constant region, for example a mouse light chain constant region, such as lambda or kappa mouse light chain region or variant thereof. Accordingly, the antibody that is obtainable from the host cell, for example a hybridoma, with the deposit number DSM ACC3121 comprises a heavy chain constant region, for example a mouse constant region, such as γ1, γ2a, γ2b, or γ3 mouse heavy chain constant region or a variant thereof. The antibody that is obtainable from the host cell, for example a hybridoma, with the deposit number DSM ACC3121 may also comprise a light chain constant region, for example a mouse light chain constant region, such as lambda or kappa mouse light chain region or variant thereof.

The invention also relates to an antibody that is obtainable from the host cell, for example a hydridoma, with the deposit number DSM ACC3175, wherein said antibody comprises a heavy chain constant region, for example a mouse constant region, such as γ1, γ2a, γ2b or γ3 mouse heavy chain constant region or a variant thereof. The antibody that is obtainable from the host cell, for example a hybridoma, with the deposit number DSM ACC3175 may also comprise a light chain constant region, for example a mouse light chain constant region, such as lambda or kappa mouse light chain region or variant thereof.

The invention also relates to an antibody that is obtainable from the host cell, for example a hydridoma, with the deposit number DSM ACC3176, wherein said antibody comprises a heavy chain constant region, for example a mouse constant region, such as γ1, γ2a, γ2b or γ3 mouse heavy chain constant region or a variant thereof. The antibody that is obtainable from the host cell, for example a hybridoma, with the deposit number DSM ACC3176 may also comprise a light chain constant region, for example a mouse light chain constant region, such as lambda or kappa mouse light chain region or variant thereof.

The invention also relates to an antibody that is obtainable from the host cell, for example a hydridoma, with the deposit number DSM ACC3177, wherein said antibody comprises a heavy chain constant region, for example a mouse constant region, such as γ1, γ2a, γ2b or γ3 mouse heavy chain constant region or a variant thereof. The antibody that is obtainable from the host cell, for example a hybridoma, with the deposit number DSM ACC3177 may also comprise a light chain constant region, for example a mouse light chain constant region, such as lambda or kappa mouse light chain region or variant thereof.

The invention also relates to an antibody that is obtainable from the host cell, for example a hydridoma, with the deposit number DSM ACC3174, wherein said antibody comprises a heavy chain constant region, for example a rat constant region, such as γ1, γ2a, γ2b or γ2c rat heavy chain constant region or a variant thereof. The antibody that is obtainable from the host cell, for example a hybridoma, with the deposit number DSM ACC3174 may also comprise a light chain constant region, for example a rat light chain constant region, such as lambda or kappa rat light chain region or variant thereof.

The term “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity of the protein. Those of skill in this art recognize that, in general, single amino acid substitutions in non-essential regions of a polypeptide do not substantially alter biological activity (see, e.g., Watson Molecular Biology of the Gene, The Benjamin/Cummings Pub. Co. 4th Ed. (1987), 224 (In addition, substitutions of structurally or functionally similar amino acids are less likely to disrupt biological activity. Within the context of the present invention the binding compounds/antibodies of the present invention comprise polypeptide chains with sequences that include up to 0 (no changes), 1, 2, 3, 4, 5, 6, 7, 8, 9, 10, 12, 15, 20 or more conservative amino acid substitutions when compared with the specific amino acid sequences disclosed herein, for example, SEQ ID NOs:4, 33, 43, 53, 63 (referring to the variable region of the antibody heavy chain of the antibody) and 6, 31, 41, 51, 61 (referring to the variable of the light chain of the antibody). As used herein, the phrase “up to X” conservative amino acid substitutions includes 0 substitutions and any number of substitutions up to 10 and including 0, 1, 2, 3, 4, 5, 6, 7, 8, 9, 10 substitutions.

Accordingly, the present invention relates to an antibody that is obtainable from/produced by a host cell, for example a hybridoma, with the deposit number DSM ACC3121 and wherein said antibody comprises a light chain variable region comprising the sequence of SEQ ID NO:6 having up to 0, 1, 2, 3, 4, 5, 6, 7, 8, 9, 10 conservative amino acid substitutions and a heavy chain variable region comprising the sequence of SEQ ID NO:4 having up to 0, 1, 2, 3, 4, 5, 6, 7, 8, 9, 10 conservative amino acid substitutions.

The present invention also relates to an antibody that is obtainable from/produced by a host cell, for example a hybridoma, with the deposit number DSM ACC3174 and wherein said antibody comprises a light chain variable region comprising the sequence of SEQ ID NO:31 having up to 0, 1, 2, 3, 4, 5, 6, 7, 8, 9, 10 conservative amino acid substitutions and a heavy chain variable region comprising the sequence of SEQ ID NO:33 having up to 0, 1, 2, 3, 4, 5, 6, 7, 8, 9, 10 conservative amino acid substitutions.

The present invention also relates to an antibody that is obtainable from/produced by a host cell, for example a hybridoma, with the deposit number DSM ACC3175 and wherein said antibody comprises a light chain variable region comprising the sequence of SEQ ID NO:41 having up to 0, 1, 2, 3, 4, 5, 6, 7, 8, 9, 10 conservative amino acid substitutions and a heavy chain variable region comprising the sequence of SEQ ID NO:43 having up to 0, 1, 2, 3, 4, 5, 6, 7, 8, 9, 10 conservative amino acid substitutions.

The present invention also relates to an antibody that is obtainable from/produced by a host cell, for example a hybridoma, with the deposit number DSM ACC3176 and wherein said antibody comprises a light chain variable region comprising the sequence of SEQ ID NO:51 having up to 0, 1, 2, 3, 4, 5, 6, 7, 8, 9, 10 conservative amino acid substitutions and a heavy chain variable region comprising the sequence of SEQ ID NO:53 having up to 0, 1, 2, 3, 4, 5, 6, 7, 8, 9, 10 conservative amino acid substitutions.

The present invention also relates to an antibody that is obtainable from/produced by a host cell, for example a hybridoma, with the deposit number DSM ACC3177 and wherein said antibody comprises a light chain variable region comprising the sequence of SEQ ID NO:61 having up to 0, 1, 2, 3, 4, 5, 6, 7, 8, 9, 10 conservative amino acid substitutions and a heavy chain variable region comprising the sequence of SEQ ID NO:63 having up to 0, 1, 2, 3, 4, 5, 6, 7, 8, 9, 10 conservative amino acid substitutions.

Such exemplary substitutions are preferably made in accordance with those set forth in Table 1 as follows:

TABLE 1
Exemplary Conservative Amino Acid Substitutions

| Original residue | Conservative substitution |
|---|---|
| Ala (A) | Gly; Ser |
| Arg (R) | Lys, His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

The present invention also relates to a nucleic acid, for example DNA, encoding an antibody of the present invention, for example an antibody that binds to the second extracellular loop of the human β1-adrenoreceptor (β1-AR-ECII). The nucleic acid encodes an antibody comprising at least one antibody light chain variable region (VL) and at least one antibody heavy chain variable region (VH), or binding fragments of these domains, wherein the VL comprises the complementarity determining regions (CDR) having the sequences CDRL1, CDRL2, CDRL3 of SEQ ID NOs:10, 11 and/or 12, respectively; and/or wherein the VH comprises the CDR having the sequences of CDRH1, CDRH2, CDRH3 of SEQ ID NOs:7, 8 and/or 9, respectively.

The nucleic acid molecule may also encode one or both of the heavy and/or light chain variable regions comprising or consisting of SEQ ID NO:4 and/or SEQ ID NO:6. The nucleic acid molecule of the present invention may also encode the antibody that is produced by/obtainable from a host cell, for example a hybridoma, with the deposit number DSM ACC3121.

The present invention also relates to a nucleic acid, for example DNA, encoding an antibody of the present invention, for example an antibody that binds to the second extracellular loop of the human β1-adrenoreceptor (β1-AR-ECII). The nucleic acid encodes an antibody comprising at least one antibody light chain variable region (VL) and at least one antibody heavy chain variable region (VH), or binding fragments of these domains, wherein the VL comprises the complementarity determining regions (CDR) having the sequences CDRL1, CDRL2, CDRL3 of SEQ ID NOs:34, 35 and/or 36, respectively; and/or wherein the VH comprises the CDR having the sequences of CDRH1, CDRH2, CDRH3 of SEQ ID NOs:37, 38 and/or 39, respectively.

The nucleic acid molecule may also encode one or both of the heavy and/or light chain variable regions comprising or consisting of SEQ ID NO:33 and/or SEQ ID NO:31. The nucleic acid molecule of the present invention may also encode the antibody that is produced by/obtainable from a host cell, for example a hybridoma, with the deposit number DSM ACC3174.

The present invention also relates to a nucleic acid, for example DNA, encoding an antibody of the present invention, for example an antibody that binds to the second extracellular loop of the human β1-adrenoreceptor (β1-AR-ECII). The nucleic acid encodes an antibody comprising at least one antibody light chain variable region (VL) and at least one antibody heavy chain variable region (VH), or binding fragments of these domains, wherein the VL comprises the complementarity determining regions (CDR) having the sequences CDRL1, CDRL2, CDRL3 of SEQ ID NOs:44, 45 and/or 46, respectively; and/or wherein the VH comprises the CDR having the sequences of CDRH1, CDRH2, CDRH3 of SEQ ID NOs:47, 48 and/or 49, respectively.

The nucleic acid molecule may also encode one or both of the heavy and/or light chain variable regions comprising or consisting of SEQ ID NO:43 and/or SEQ ID NO:41. The nucleic acid molecule of the present invention may also encode the antibody that is produced by/obtainable from a host cell, for example a hybridoma, with the deposit number DSM ACC3175.

The present invention also relates to a nucleic acid, for example DNA, encoding an antibody of the present invention, for example an antibody that binds to the second extracellular loop of the human β1-adrenoreceptor (β1-AR-ECII). The nucleic acid encodes an antibody comprising at least one antibody light chain variable region (VL) and at least one antibody heavy chain variable region (VH), or binding fragments of these domains, wherein the VL comprises the complementarity determining regions (CDR) having the sequences CDRL1, CDRL2, CDRL3 of SEQ ID NOs:54, 55 and/or 56, respectively; and/or wherein the VH comprises the CDR having the sequences of CDRH1, CDRH2, CDRH3 of SEQ ID NOs:57, 58 and/or 59, respectively.

The nucleic acid molecule may also encode one or both of the heavy and/or light chain variable regions comprising or consisting of SEQ ID NO:53 and/or SEQ ID NO:51. The nucleic acid molecule of the present invention may also encode the antibody that is produced by/obtainable from a host cell, for example a hybridoma, with the deposit number DSM ACC3176.

The present invention also relates to a nucleic acid, for example DNA, encoding an antibody of the present invention, for example an antibody that binds to the second extracellular loop of the human β1-adrenoreceptor (β1-AR-ECII). The nucleic acid encodes an antibody comprising at least one antibody light chain variable region (VL) and at least one antibody heavy chain variable region (VH), or binding fragments of these domains, wherein the VL comprises the complementarity determining regions (CDR) having the sequences CDRL1, CDRL2, CDRL3 of SEQ ID NOs:64, 65 and/or 66, respectively; and/or wherein the VH comprises the CDR having the sequences of CDRH1, CDRH2, CDRH3 of SEQ ID NOs:67, 68 and/or 69, respectively.

The nucleic acid molecule may also encode one or both of the heavy and/or light chain variable regions comprising or consisting of SEQ ID NO:63 and/or SEQ ID NO:61. The nucleic acid molecule of the present invention may also encode the antibody that is produced by/obtainable from a host cell, for example a hybridoma, with the deposit number DSM ACC3177.

Said nucleic acid molecule may be a naturally nucleic acid molecule as well as a recombinant nucleic acid molecule. The nucleic acid molecule of the invention may, therefore, be of natural origin, synthetic or semi-synthetic. It may comprise DNA, RNA as well as PNA and it may be a hybrid thereof.

It is evident to the person skilled in the art that regulatory sequences may be added to the nucleic acid molecule of the invention. For example, promoters, transcriptional enhancers and/or sequences which allow for induced expression of the polynucleotide of the invention may be employed. A suitable inducible system is for example tetracycline-regulated gene expression as described, e.g., by Gossen and Bujard, Proc. Natl. Acad. Sci. USA 89 (1992), 5547-5551) and Gossen, Trends Biotech. 12 (1994), 58-62, or a dexamethasone-inducible gene expression system as described, e.g. by Crook, EMBO J. 8 (1989), 513-519.

Furthermore, said nucleic acid molecule may contain, for example, thioester bonds and/or nucleotide analogues. Said modifications may be useful for the stabilization of the nucleic acid molecule against endo- and/or exonucleases in the cell. Said nucleic acid molecules may be transcribed by an appropriate vector containing a chimeric gene which allows for the transcription of said nucleic acid molecule in the cell. In this respect, it is also to be understood that the nucleic acid molecule encoding the binding compound/antibody of the present invention can be used for “gene targeting”. In the context of the present invention said nucleic acid molecules are labeled. Methods for the detection of nucleic acids are well known in the art, e.g., Southern and Northern blotting, PCR or primer extension.

The nucleic acid molecule(s) of the invention may be a recombinantly produced chimeric nucleic acid molecule comprising any of the aforementioned nucleic acid molecules either alone or in combination. Preferably, the nucleic acid molecule of the invention is part of a vector.

The present invention therefore also relates to a vector comprising the nucleic acid molecule of the present invention. Accordingly, the present invention relates to vectors, preferably expression vectors comprising the nucleic acids of the invention.

The vector of the present invention may be, e.g., a plasmid, cosmid, virus, bacteriophage or another vector used e.g. conventionally in genetic engineering, and may comprise further genes such as marker genes which allow for the selection of said vector in a suitable host cell and under suitable conditions.

Furthermore, the vector of the present invention may, in addition to the nucleic acid sequences of the invention, comprise expression control elements, allowing proper expression of the coding regions in suitable hosts. Such control elements are known to the skilled person and may include a promoter, a splice cassette, translation initiation codon, translation and insertion site for introducing an insert into the vector. Preferably, the nucleic acid molecule of the invention is operatively linked to said expression control sequences allowing expression in eukaryotic or prokaryotic cells. Accordingly, the present invention relates to a vector comprising the nucleic acids of the invention, wherein the nucleic acid is operably linked to control sequences that are recognized by a host cell when the eukaryotic and/or prokaryotic (host) cell is transfected with the vector.

Control elements ensuring expression in eukaryotic and prokaryotic (host) cells are well known to those skilled in the art. As mentioned herein above, they usually comprise regulatory sequences ensuring initiation of transcription and optionally poly-A signals ensuring termination of transcription and stabilization of the transcript. Additional regulatory elements may include transcriptional as well as translational enhancers, and/or naturally-associated or heterologous promoter regions. Possible regulatory elements permitting expression in for example mammalian host cells comprise the CMV-HSV thymidine kinase promoter, SV40, RSV-promoter (Rous Sarcoma Virus), human elongation factor 1α-promoter, the glucocorticoid-inducible MMTV-promoter (Moloney Mouse Tumor Virus), metallothionein- or tetracyclin-inducible promoters, or enhancers, like CMV enhancer or SV40-enhancer. For expression in neural cells, it is envisaged that neurofilament-, PGDF-, NSE-, PrP-, or thy-1-promoters can be employed. Said promoters are known in the art and, inter alia, described in Charron J. Biol. Chem. 270 (1995), 25739-25745. For the expression in prokaryotic cells, a multitude of promoters including, for example, the tac-lac-promoter or the trp promoter, has been described. Besides elements which are responsible for the initiation of transcription such regulatory elements may also comprise transcription termination signals, such as SV40-poly-A site or the tk-poly-A site, downstream of the polynucleotide. In this context, suitable expression vectors are known in the art such as Okayama-Berg cDNA expression vector pcDV1 (Pharmacia), pRc/CMV, pcDNA1, pcDNA3 (In-vitrogene), pSPORT1 (GIBCO BRL), pX (Pagano, Science 255 (1992), 1144-1147), yeast two-hybrid vectors, such as pEG202 and dpJG4-5 (Gyuris, Cell 75 (1995), 791-803), or prokaryotic expression vectors, such as lambda gt11 or pGEX (Amersham-Pharmacia). Beside the nucleic acid molecules of the present invention, the vector may further comprise nucleic acid sequences encoding for secretion signals. Such sequences are well known to the person skilled in the art. Furthermore, depending on the expression system used leader sequences capable of directing the peptides of the invention to a cellular compartment may be added to the coding sequence of the nucleic acid molecules of the invention and are well known in the art. The leader sequence(s) is (are) assembled in appropriate phase with translation, initiation and termination sequences, and preferably, a leader sequence capable of directing secretion of translated protein, or a protein thereof, into the periplasmic space or extracellular medium. Optionally, the heterologous sequence can encode a fusion protein including a C- or N-terminal identification peptide imparting desired characteristics, e.g., stabilization or simplified purification of expressed recombinant product. Once the vector has been incorporated into the appropriate host, the host is maintained under conditions suitable for high level expression of the nucleotide sequences, and, as desired, the collection and purification of the antibody molecules or fragments thereof of the invention may follow.

Furthermore, the vector of the present invention may also be an expression vector. The nucleic acid molecules and vectors of the invention may be designed for direct introduction or for introduction via liposomes, viral vectors (e.g. adenoviral, retroviral), electroporation, ballistic (e.g. gene gun) or other delivery systems into the cell. Additionally, a baculoviral system can be used as eukaryotic expression system for the nucleic acid molecules of the invention.

The present invention also relates to a host cell transfected or transformed with the vector of the invention or a non-human host carrying the vector of the present invention, i.e. to a host cell or host which is genetically modified with a nucleic acid molecule according to the invention or with a vector comprising such a nucleic acid molecule. The term “genetically modified” means that the host cell or host comprises in addition to its natural genome a nucleic acid molecule or vector according to the invention which was introduced into the cell or host or into one of its predecessors/parents. The nucleic acid molecule or vector may be present in the genetically modified host cell or host either as an independent molecule outside the genome, preferably as a molecule which is capable of replication, or it may be stably integrated into the genome of the host cell or host.

The host cell of the present invention may be any prokaryotic or eukaryotic cell. Suitable prokaryotic cells are those generally used for cloning like E. coli or Bacillus subtilis. Furthermore, eukaryotic cells comprise, for example, fungal or animal cells. Examples for suitable fungal cells are yeast cells, preferably those of the genus Saccharomyces and most preferably those of the species Saccharomyces cerevisiae. Suitable animal cells are, for instance, insect cells, vertebrate cells, preferably mammalian cells, such as e.g. HEK293, NSO, CHO, MDCK, U2-OSHela, NIH3T3, MOLT-4, Jurkat, PC-12, PC-3, IMR, NT2N, Sk-n-sh, CaSki, C33A. These host cells, e.g. CHO-cells, may provide posts-translational (secondary) modifications to the antibody molecules of the invention, including leader peptide removal, folding and assembly of H and C chains, glycosylation of the molecule at correct sides and secretion of the functional molecule. Further suitable cell lines known in the art are obtainable from cell line depositories, like, e.g., the Deutsche Sammlung von Mikroorganismen and Zellkulturen GmbH (DSMZ) or the American Type Culture Collection (ATCC). In accordance with the present invention, it is furthermore envisaged that primary cells/cell cultures may function as host cells. Said cells are in particular derived from insects (like insects of the species Drosophila or Blatta) or mammals (like human, swine, mouse or rat). Said host cells may also comprise cells from and/or derived from cell lines like neuroblastoma cell lines. The above mentioned primary cells are well known in the art and comprise, inter alia, primary astrocytes, (mixed) spinal cultures or hippocampal cultures.

In the context of the present invention, the host cell of the present invention may be a hybridoma having the accession number DSM ACC3121. Accordingly, the present invention relates to a host cell, for example a hybridoma, having the deposit number DSM ACC3121, which produces the binding molecule of the present invention. The present invention also relates to a host cell, for example a hybridoma, having the deposit number DSM ACC3174. The invention also relates to a host cell, for example a hybridoma, having the deposit number DSM ACC3175. The invention also relates to a host cell, for example a hybridoma, having the deposit number DSM ACC3176. The invention also relates to a host cell, for example a hybridoma, having the deposit number DSM ACC3177.

Host cells, for example hybridomas, producing (monoclonal) antibodies that bind against the second extracellular loop of the human β1-adrenoreceptor (β1-AR-ECII) has been deposited by the Corimmun GmbH, Fraunhoferstr. 17, 82152 Martinsried at the DSMZ-Deutsche Sammlung von Mikroorganismen und Zellkulturen GmbH, Inhoffenstr. 7B, D-38124 Braunschweig, Germany.

Hybridoma (23-6-7) producing a (mouse monoclonal) antibody which binds against the second extracellular loop of the human β1-adrenoreceptor (β1-AR-ECII) has been deposited by the Corimmun GmbH, Fraunhoferstr. 17, 82152 Martinsried at the DSMZ-Deutsche Sammlung von Mikroorganismen und Zellkulturen GmbH, Inhoffenstr. 7B, D-38124 Braunschweig, Germany on Mar. 15, 2011. The deposit name and the DSM accession number for the hybridoma is “b1ECII E3, 23-6-7 (anti-beta1-AR)” and “DSM ACC3121 (DSMZ ACC3121)”.

Hybridoma (28-2-7) producing a (mouse monoclonal) antibody that binds against the second extracellular loop of the human β1-adrenoreceptor (β1-AR-ECII) has been deposited by the Corimmun GmbH, Fraunhoferstr. 17, D-82152 Martinsried at the DSMZ-Deutsche Sammlung von Mikroorganismen und Zellkulturen GmbH, Inhoffenstr. 7B, D-38124 Braunschweig, Germany on May 16, 2012. The deposit name and the DSM accession number for the hybridoma (23-6-7) is “b1ECII, 28-2-7” and “DSM ACC3175 (DSMZ ACC3175)”.

Hybridoma (47-12-9) producing a (mouse monoclonal) antibody that binds against the second extracellular loop of the human β1-adrenoreceptor (β1-AR-ECII) has been deposited by the Corimmun GmbH, Fraunhoferstr. 17, D-82152 Martinsried at the DSMZ-Deutsche Sammlung von Mikroorganismen und Zellkulturen GmbH, Inhoffenstr. 7B, D-38124 Braunschweig, Germany on May 16, 2012. The deposit name and the DSM accession number for the hybridoma (47-12-9) is “b1ECII, 47-12-9” and “DSM ACC3176 (DSMZ ACC3176)”.

Hybridoma (50-1-5) producing a (mouse monoclonal) antibody that binds against the second extracellular loop of the human β1-adrenoreceptor (β1-AR-ECII) has been deposited by the Corimmun GmbH, Fraunhoferstr. 17, D-82152 Martinsried at the DSMZ-Deutsche Sammlung von Mikroorganismen und Zellkulturen GmbH, Inhoffenstr. 7B, D-38124 Braunschweig, Germany on May 16, 2012. The deposit name and the DSM accession number for the hybridoma (50-1-5) is “b1ECII, 50-1-5” and “DSM ACC3177 (DSMZ ACC3177)”.

Hybridoma (13/F6) producing a (rat monoclonal) antibody that binds against the second extracellular loop of the human β1-adrenoreceptor (β1-AR-ECII) has been deposited by the Corimmun GmbH, Fraunhoferstr. 17, D-82152 Martinsried at the DSMZ-Deutsche Sammlung von Mikroorganismen und Zellkulturen GmbH, Inhoffenstr. 7B, D-38124 Braunschweig, Germany on May 16, 2012. The deposit name and the DSM accession number for the hybridoma (host cell) expressing the rat monoclonal antibody (clone) 13F6 is “13/F6” and “DSM ACC3174 (DSMZ ACC3174)”.

The present invention relates to methods of producing a binding compound/antibody of the present invention culturing a host cell harbouring an expression vector encoding the binding compounds in culture medium, and recovering the binding compound/antibody from the host cell or culture medium. The present invention may also relate to a method for producing an antibody of the present invention comprising the cultivation of the host cell of the present invention and recovering the binding compound from the culture. Accordingly, the present invention relates to a method for producing an antibody of the present invention, wherein said method comprises the cultivation of the host cell, for example a hybridoma, with the deposit number DSM ACC3121 and recovering the antibody that is obtainable from the host cell, for example a hybridoma, with the deposit number DSM ACC3121 from the medium. The invention also relates to a method for producing an antibody of the present invention, wherein said method comprises the cultivation of the host cell, for example a hybridoma, with the deposit number DSM ACC3174 and recovering the antibody that is obtainable from the host cell, for example a hybridoma, with the deposit number DSM ACC3174 from the medium. The invention also relates to a method for producing an antibody of the present invention, wherein said method comprises the cultivation of the host cell, for example a hybridoma, with the deposit number DSM ACC3175 and recovering the antibody that is obtainable from the host cell, for example a hybridoma, with the deposit number DSM ACC3175 from the medium. The invention also relates to a method for producing an antibody of the present invention, wherein said method comprises the cultivation of the host cell, for example a hybridoma, with the deposit number DSM ACC3176 and recovering the antibody that is obtainable from the host cell, for example a hybridoma, with the deposit number DSM ACC3176 from the medium. The invention also relates to a method for producing an antibody of the present invention, wherein said method comprises the cultivation of the host cell, for example a hybridoma, with the deposit number DSM ACC3177 and recovering the antibody that is obtainable from the host cell, for example a hybridoma, with the deposit number DSM ACC3177 from the medium.

Since host cells, e.g., CHO cells, may provide post-translational (secondary) modification on the expressed binding compounds of the present invention. These modifications comprise, inter alia, glycosylation and phosphorylation. Accordingly, the present invention also relates to antibodies that bind to the second extracellular loop of the human β1-adrenoreceptor produced by the host cells of the present invention. Accordingly, in the context of the present invention the binding compound/antibody is produced by the hybridoma as deposited under DSM ACC3121.

The present invention relates to binding compounds, such as antibodies or fragments thereof, that bind to the same epitope on the second extracellular loop of the human β1-adrenoreceptor (β1-AR-ECII) as binding compounds obtainable from/produced by host cells as described above and/or obtainable from/produced by a hybridoma with a deposit number of DSM ACC3121. The present invention relates to binding compounds, such as antibodies or binding fragments thereof, that bind to the same epitope on the second extracellular loop of the human β1-adrenoreceptor (β1-AR-ECII) as binding compounds obtainable from/produced by host cells as described above and/or obtainable from/produced by a hybridoma with a deposit number of DSM ACC3174. The present invention relates to binding compounds, such as antibodies or binding fragments thereof, that bind to the same epitope on the second extracellular loop of the human β1-adrenoreceptor (β1-AR-ECII) as binding compounds obtainable from/produced by host cells as described above and/or obtainable from/produced by a hybridoma with a deposit number of DSM ACC3175. The present invention relates to binding compounds, such as antibodies or binding fragments thereof, that bind to the same epitope on the second extracellular loop of the human β1-adrenoreceptor (β1-AR-ECII) as binding compounds obtainable from/produced by host cells as described above and/or obtainable from/produced by a hybridoma with a deposit number of DSM ACC3176. The present invention relates to binding compounds, such as antibodies or binding fragments thereof, that bind to the same epitope on the second extracellular loop of the human β1-adrenoreceptor (β1-AR-ECII) as binding compounds obtainable from/produced by host cells as described above and/or obtainable from/produced by a hybridoma with a deposit number of DSM ACC3177.

The invention relates to antibodies that bind to the second extracellular loop of the human β1-adrenoreceptor (β1-AR-ECII) or fragments thereof, such as antibodies that bind with equilibrium dissociation constants (K_{d}) of 1000, 900, 800, 700, 600, 550, 540, 530, 520, 510, 500, 490, 480, 470, 460, 450, 440, 430, 420, 410, 400, 390, 380, 370, 360, 350 pM or less. The present invention also relates to antibodies or fragments thereof that are obtainable from a host cell with the deposit number DSM ACC3121 that bind to the second extracellular loop of the human β1-AR-ECII or binding fragments thereof, wherein an antibody or a fragment thereof that is obtainable from a host cell with deposit number DSM ACC3121 is characterized by having an equilibrium dissociation constants (K_{d}) of 1000, 900, 800, 700, 600, 550, 540, 530, 520, 510, 500, 490, 480, 470, 460, 450, 440, 430, 420, 410, 400, 390, 380, 370, 360, 350 pM or less. The invention also relates to an antibody or a fragment thereof that is obtainable from the host cell with the deposit number DSM ACC3121 and wherein said antibody binds to the second extracellular loop of the human β₁-adrenoreceptor with an equilibrium dissociation constant (K_{d}) of 510 pM or less.

The binding compounds of the present invention may also be antibodies or fragments thereof that bind to second extracellular loop of the human β1-adrenoreceptor (β₁-AR-ECII) with an affinity (K_{d}) that is at least 1000, 100, 50, 40, 30, 20, 10, 5-fold lower compared to the rat monoclonal antibody 13F6 that is obtainable from the host cell (hybridoma) with the deposit number DSM ACC3174 or goat polyclonal antibodies that bind to the second extracellular loop of the human β1-adrenoreceptor (β1-AR-ECII). The invention also relates to antibodies or fragments thereof that are obtainable from the host cell (hybridoma) with the deposit number DSM ACC3121 or fragments thereof that bind to second extracellular loop of the human β1-adrenoreceptor (β₁-AR-ECII) with an affinity (K_{d}) that is at least 1000, 100, 50, 40, 30, 20, 10, 5-fold lower compared to the rat monoclonal antibody 13F6 that is obtainable from the host cell (hybridoma) with the deposit number DSM ACC3174 or goat polyclonal antibodies that bind to the second extracellular loop of the human β1-adrenoreceptor (β1-AR-ECII).

The binding compounds of the present invention may also be antibodies or fragments thereof that have an IC50 value of 2000, 1900, 1800, 1700, 1600, 1500, 1400, 1300, 1200, 1100, 1000, 900, 800, 700, 600, 500, 400, 300, 200, 100, 50, 10 pM or less when measured in a biological assay system where the binding affinity to the second extracellular loop of the human β1-adrenoreceptor (β₁-AR-ECII) is measured in the presence of (an) receptor homologous of the (human) β1-adrenoreceptor. The invention also relates to antibodies or fragments thereof that are obtainable from the host cell, for example a hybridoma, with the deposit number DSM ACC3121 that have an IC50 value of 2000, 1900, 1800, 1700, 1600, 1500, 1400, 1300, 1200, 1100, 1000, 900, 800, 700, 600, 500, 400, 300, 200, 100, 50, 10 pM or less when measured in a biological assay system where the binding affinity to the second extracellular loop of the human β1-adrenoreceptor (β₁-AR-ECII) is measured in the presence of (an) receptor homologous of the (human)β1-adrenoreceptor.

Accordingly, the present invention relates to the antibody or fragments thereof that are obtainable from the host cell, for example a hybridoma, with the deposit number DSM ACC3121, wherein said antibody has at least one of the following properties:
(a) the antibody binds to the second extracellular loop of the human β₁-adrenoreceptor with an equilibrium dissociation constant (K_{d}) of 1000 pM or less;
(b) the binding affinity to the second extracellular loop of the human β1-adrenoreceptor is competitively inhibited with an IC50 value of 2000 pM or less in the presence of (an) receptor homologous of the (human) β1-adrenoreceptor; and/or
(c) the binds to the second extracellular loop of the human β₁-adrenoreceptor with an affinity (K_{d}) that is at least 10-fold lower compared to rat monoclonal antibodies, preferably to the rat monoclonal antibody that is obtainable from the host cell with the deposit number DSM ACC3174, or goat polyclonal antibodies that bind to the second extracellular loop of the human β₁-adrenoreceptor.

Antibodies or fragments thereof that are obtainable from the host cell, for example a hybridome, with the deposit number DSM ACC3121 having the characteristics identified herein can be screened for example by measuring binding affinity. To screen for antibodies that bind the same epitope on the second extracellular loop of the human β1-adrenoreceptor (β1-AR) bound by an antibody that is obtainable from a host cell, for example a hybridoma, with the deposit number selected from the group consisting of DSM ACC3121, DSM ACC3174, DSM ACC3175, DSM ACC3176 and DSM ACC3177 a routine cross-blocking assay can be performed such as that is described in Antibodies, A Laboratory Manual Cold Spring Harbor Laboratory, Ed Harlow and David Lane (1988). Alternatively, epitope mapping can be performed by alanine permutation scanning or, for example, by methods as described in Champe, J. Biol. Chem. 270 (1995), 1388-1394. Antibody affinity, for example for the second extracellular loop of the human β1-AR, can be determined by using standard methods, including those described in the appended Examples. Preferred antibodies or fragments thereof are those which bind the second extracellular loop of the β1-AR with an equilibrium dissociation constant (K_{d}) of 1000 pM or less. Even more preferred are antibodies or fragments thereof that have K_{d} values of no more than about 510 pM.

β1-receptor homologous as used in the present invention may include, inter alia, molecules, substances or compounds of chemical or biological origin, molecules, substances or compounds found in nature or being synthetically, recombinantly and/or chemically produced. Specifically, the β1-receptor homologous are receptor homologous to the human β1-adrenoreceptor. Specifically, the β1-receptor homologous are peptides or cyclo-peptides having a sequence similarity to the first (β1-ECI), the second (β1-ECII) or the third extracellular loop (β1-ECIII) of a β1-adrenoreceptor, preferably the human β1-adrenoreceptor. The third extracellular domain (β1-ECIII) of a β1-adrenoreceptor contains or consists of the amino acid sequence Lys-Ala-Phe-His-Arg-Glu-Leu-Val-Pro-Asp-Arg. The peptides or cylo-peptides having sequence similarity against the second (β1-ECII) extracellular loop of the (human) β1-adrenoreceptor comprise or consist the general formula (x-xₕ-Cys-x-x^{a}-x^{b}-x^{c}-x-Cys-y-xᵢ-x) or cyclo (x-xₕ-Cys-x-x^{a}-x^{b}-x^{c}-x-Cys-y-xᵢ-x). In this formula, the term “y” can be any amino acid except Cys, preferably “y” can be any amino acid except Cys and/or Pro. Generally, “y” can be any amino acid, as long as this amino acid has no intramolecular link (e.g., a disulfide bond) with another amino acid of the herein described cyclo-peptide (e.g., a different Cys of the herein described cyclo-peptide). Preferably, “y” can be any amino acid that is similar to Cys (i.e., an amino acid that have a similar chemical structure and/or similar biochemical behavior as Cys has), except that there is no intra-molecular link (e.g., a disulfide bond) with another amino acid of a herein described cyclo-peptide (e.g., with another Cys of a cyclo-peptide described herein) or inter-molecular link with endogenous cellular proteins that a contain Cys residue. Preferably, “y” can be any polar amino acid, with the exception of Cys or Thr. Specifically, in the herein described cyclo-peptide “y” can be Ser. In the context of the present invention, “y” can be selenocysteine or an analogue thereof. Furthermore, within the context of the present invention, “y” can be alpha-butyric acid (Abu) or Abu analogue. Examples of suitable (cyclo-) peptides are: (cyclo) (Arg-Ala-Glu-Ser-Asp-Glu-Ala-Arg-Arg-Cys-Tyr-Asn-Asp-Pro-Lys-Cys-Abu-Asp-Phe-Val-Thr-Gly) referring to SEQ ID NO:13, (cyclo) (Ala-Asp-Glu-Ala-Arg-Arg-Cys-Tyr-Asn-Asp-Pro-Lys-Cys-Abu-Asp-Phe-Val-Gln) referring to SEQ ID NO:14, and (cyclo) (Ala-Arg-Ala-Glu-Ser-Asp-Glu-Ala-Arg-Arg-Cys-Tyr-Asn-Asp-Pro-Lys-Cys-Abu-Asp-Phe-Val-Thr-Asn-Arg-Gln) referring to SEQ ID NO:15. In the context described herein, in the (cyclo-) peptides (see above formulas) “h” can be a number from 1 to 15, preferably 5 to 9, and/or “i” can be a number from 0 to 14, preferably 1 to 14. Accordingly, in the context described herein, “i” can be a number between 0 to 6, preferably 1 to 6. Accordingly, “h” can be 1, 2, 3, 4, 5, 6, 7, 8, 9, 10, 11, 12, 13, 14 or 15 and/or “i” can be 0, 1, 2, 3, 4, 5, 6, 7, 8, 9, 10, 11, 12, 13 and 14. Preferably “h” is 5 or 9 or “i” is 3 or 6. Furthermore, in the context of the invention “xₕ” can be the amino acid sequence Asp-Glu-Ala-Arg-Arg or Arg-Ala-Glu-Ser-Asp-Glu-Ala-Arg-Arg and/or “xᵢ” is the amino acid sequence Asp-Phe-Val, Asp-Phe-Val-Thr or Asp-Phe-Val-Thr-Asn-Thr. In the context of the present invention, “xₕ” is the amino acid sequence Ala-Glu-Ser-Asp-Glu-Ala-Arg-Arg and/or “xᵢ” is the amino acid sequence DFVT. Furthermore, the (cylco-) peptide (or the cyclic part thereof) as described in the present invention, includes only one Pro. Accordingly, it is preferred that neither “y” or “x”, other than by exactly one of x^{a}, x^{b} and x^{c}, is Pro. Within the context of the present invention, “x^{c}” is Pro and “x^{b}”, as herein described in any of the above formulas, is an acidic amino acid such as Asp or Glu. For example, if “x^{c}” is Pro, “x^{a}” can be an acidic amino acid, and if “xₐ” is Pro, “x”, as described herein in any of the above formulas, which is located between “xₐ” and the first Cys, can be an acidic amino acid.

The (cyclo-) peptide (or the cyclic part thereof), as described in the present invention, comprises 18 to 25 amino acids. Accordingly, the (cyclo-) peptide of the present invention comprises 18, 19, 20, 21, 23, 24 or 25 amino acids, wherein the (cyclo-) peptide preferably comprises 18, 22 or 25, or more preferably comprises 18 or 22 amino acids. In the context of the present invention, the (cyclo-) peptide (or the cyclic part thereof) comprises fewer amino acids, e.g., 16 or 17 amino acids. In the context of the present invention, the herein described β1-receptor homologous can be, mutatis mutandis, linear peptides. The herein described β1-receptor homologous can also be (cyclo-) peptides, which have a sequence similarity with the third extracellular loop of the (human) β1-adrenoreceptor (see above). β1-receptor homologous are well known in the art and described, inter alia, in WO 2006/103101 and WO 2009/027063. The β1-receptor homologous as disclosed in WO 2006/103101 and WO 2009/027063 are within the context of the present invention. Particularly, preferred are the (cyclo-) peptides as described in WO 2009/027063. In the context of the present invention, the β1-receptor homologous preferably refers to the amino acid sequence (peptide) as depicted in SEQ ID NO:16, referring to cyclo (Ala-Asp-Glu-Ala-Arg-Arg-Cys-Tyr-Asn-Asp-Pro-Lys-Cys-Ser-Asp-Phe-Val-Gln).

In context of this invention, an intramolecular S-S linkage within the cyclic (cyclo) peptide provided can be formed between two Cys residues within the amino acid backbone/primary amino acid sequence of said cyclic (cyclo) peptide as described herein. In the context of the present invention the β1-receptor homologous refers to the amino acid sequence (peptide) as depicted in SEQ ID NO:16, referring to cyclo (Ala-Asp-Glu-Ala-Arg-Arg-Cys-Tyr-Asn-Asp-Pro-Lys-Cys-Ser-Asp-Phe-Val-Gln) with an intramolecular S-S linkage between the two Cys residues. In this cyclic (cyclo) peptide (i.e., SEQ ID NO:16), referring to a homologous to an ECII epitope of the human β1-AR, cyclization may occur between Ala₁ and Gln₁₈.

Accordingly, as shown in the appended Examples of the present invention, the antibodies described herein may also be antibodies or fragments thereof that have an IC50 value of 2000, 1900, 1800, 1700, 1600, 1500, 1400, 1300, 1200, 1100, 1000, 900, 800, 700, 600, 500, 400, 300, 200, 100, 50, 10 pM or less when measured in a biological assay system where the binding affinity to the second extracellular loop of the human β1-adrenoreceptor (β1-AR-ECII) is measured in the presence of the peptide cyclo (Ala-Asp-Glu-Ala-Arg-Arg-Cys-Tyr-Asn-Asp-Pro-Lys-Cys-Ser-Asp-Phe-Val-Gln) (as depicted in SEQ ID NO:16). The invention also relates to antibodies or fragments thereof that are obtainable from the host cell, for example a hybridoma, with the deposit number DSM ACC3121 that have an IC50 value of 2000, 1900, 1800, 1700, 1600, 1500, 1400, 1300, 1200, 1100, 1000, 900, 800, 700, 600, 500, 400, 300, 200, 100, 50, 10 pM or less when measured in a biological assay system where the binding affinity to the second extracellular loop of the human β1-adrenoreceptor ECII) is measured in the presence of peptide cyclo (Ala-Asp-Glu-Ala-Arg-Arg-Cys-Tyr-Asn-Asp-Pro-Lys-Cys-Ser-Asp-Phe-Val-Gln) (as depicted in SEQ ID NO:16).

Accordingly, the present invention relates to the antibody or fragments thereof that are obtainable from the host cell (hybridoma) with the deposit number DSM ACC3121, wherein said antibody has at least one of the following properties:
(a) the antibody binds to the second extracellular loop of the human β₁-adrenoreceptor with an equilibrium dissociation constant (K_{d}) of 1000 pM or less;
(b) the binding affinity to the second extracellular loop of the human β1-adrenoreceptor is competitively inhibited with an IC50 value of 2000 pM or less in the presence of the peptide cyclo (Ala-Asp-Glu-Ala-Arg-Arg-Cys-Tyr-Asn-Asp-Pro-Lys-Cys-Ser-Asp-Phe-Val-Gln) (as depicted in SEQ ID NO:16); and/or
(c) the binds to the second extracellular loop of the human β₁-adrenoreceptor with an affinity (K_{d}) that is at least 10-fold lower compared to rat monoclonal antibodies, preferably to the rat monoclonal antibody that is obtainable from the host cell with the deposit number DSM ACC3174, or goat polyclonal antibodies that bind to the second extracellular loop of the human β₁-adrenoreceptor.

The invention also relates to antibodies or fragments thereof that are obtainable from the host cell (hybridoma) with the deposit number DSM ACC3121, that have an IC50 value of 1200 1100, 1000, 900, 800, 700, 600, 500, 400, 300, 200, 100, 50, 10 pM or less when measured in a biological assay system where the binding affinity to the second extracellular loop of the human β1-adrenoreceptor (β₁-AR-ECII) is measured in the presence of the peptide cyclo (Ala-Asp-Glu-Ala-Arg-Arg-Cys-Tyr-Asn-Asp-Pro-Lys-Cys-Ser-Asp-Phe-Val-Gln) (as depicted in SEQ ID NO:16).

Furthermore, as illustrated in the appended examples, the antibodies of the present invention are useful as a diagnostic agent/diagnostic reagent in the detection of molecule(s) or compound(s) in a biological sample. Accordingly, the invention relates to the antibody or fragments thereof that are produced by/obtainable from the host cell, for example a hydriboma, with the deposit number DSM ACC3121 that is (are) useful as a diagnostic agent/diagnostic reagent in the detection of molecule(s) or compound(s) in a biological sample. The invention also relates to the antibody or fragments thereof that are obtainable from the host cell, for example a hydriboma, with the deposit number DSM ACC3174 that is (are) useful as a diagnostic agent/diagnostic reagent in the detection of molecule(s) or compound(s) in a biological sample. The invention also relates to the antibody or fragments thereof that are obtainable from the host cell, for example a hydriboma, with the deposit number DSM ACC3175 that is (are) useful as a diagnostic agent/diagnostic reagent in the detection of molecule(s) or compound(s) in a biological sample. The invention also relates to the antibody or fragments thereof that are obtainable from the host cell, for example a hydriboma, with the deposit number DSM ACC3176 that is (are) useful as a diagnostic agent/diagnostic reagent in the detection of molecule(s) or compound(s) in a biological sample. The invention also relates to the antibody or fragments thereof that are obtainable from the host cell, for example a hydriboma, with the deposit number DSM ACC3177 that is (are) useful as a diagnostic agent/diagnostic reagent in the detection of molecule(s) or compound(s) in a biological sample.

The biological sample, as defined herein, may be, for example, a cell, a cell lysate, a crude extract of cells, a membrane preparation tissue or biofluids. Biofluids as used herein sample in which the molecule(s) or compound(s) are detected refer preferably, o semen, lymph, serum, plasma, urine, synovial fluid or spinal fluid. The invention also relates to an embodiment, wherein the biological sample in which the molecule(s) or compound(s) are detected refer to blood, serum or plasma.

In the context of the present invention, the biological sample in the present invention comprises molecule(s) or compound(s) which are selected from antibodies, protein, protein-fragments, peptides, amino acids and/or derivates thereof.

As used herein, the molecule(s) or compound(s) refer herein to (an) antibody (antibodies) in the biological sample, preferably in blood, serum or plasma.

Furthermore in the context of the present invention, the antibody or antibodies in the biological sample refer to auto-anti-β1-adrenergic antibody (antibodies)/auto-anti-β1-AR antibody (antibodies).

Accordingly, the present invention refers to diagnostic agent/diagnostic reagent which comprises an antibody of the present invention in the detection of auto anti-β1-adrenergic antibody (antibodies)/auto-anti-β1-AR antibody (antibodies) in the blood, serum or plasma. In the context of the present invention it is preferred that said antibody which can be used as a diagnostic agent/diagnostic reagent refers to the antibody or fragments thereof that are produced by/obtainable from the host cell, for example a hydridoma, with the deposit number DSM ACC3121. In the context of the present invention the antibody which can be used as a diagnostic agent/diagnostic reagent refers to the antibody or fragments thereof that are produced by/obtainable from the host cell, for example a hydridoma, with the deposit number DSM ACC3174. In the context of the present invention the antibody which can be used as a diagnostic agent/diagnostic reagent refers to the antibody or fragments thereof that are produced by/obtainable from the host cell, for example a hydridoma, with the deposit number DSM ACC3175. In the context of the present invention the antibody which can be used as a diagnostic agent/diagnostic reagent refers to the antibody or fragments thereof that are produced by/obtainable from the host cell, for example a hydridoma, with the deposit number DSM ACC3176. In the context of the present invention the antibody which can be used as a diagnostic agent/diagnostic reagent refers to the antibody or fragments thereof that are produced by/obtainable from the host cell, for example a hydridoma, with the deposit number DSM ACC3177.

In the present invention, it is preferred that said antibodies that are obtainable from the host cell, for example a hybridoma, with the deposit number selected from the group consisting of DSM ACC3121, DSM ACC3174, DSM ACC3175, DSM ACC3176 and DSM ACC2177 of the present invention to be employed as a diagnostic agent/diagnostic reagent are detectably labeled. A variety of techniques are available for labeling biomolecules (binding compounds), are well known to the skilled person in the art and are considered to be within the scope of the present invention. Such techniques are, e.g., described in Tijssen, “Practice and theory of enzyme immuno assays”, Burden, R H and von Knippenburg (Eds), 15 (1985), “Basic methods in molecular biology”; Davis L G, Dibmer M D; Battey Elsevier (1990), Mayer et al., (Eds) “Immunochemical methods in cell and molecular biology” Academic Press, London (1987), or in the series “Methods in Enzymology”, Academic Press, Inc.

There are many different labels and methods of labeling known to those of ordinary skill in the art. Examples of the types of labels which can be used in the present invention include enzymes, radioisotopes, colloidal metals, fluorescent compounds, chemiluminescent compounds, and bioluminescent compounds.

Commonly used labels comprise, inter alia, fluorochromes (like fluorescein, rhodamine, Texas Red, etc.), enzymes (like horse radish peroxidase, β-galactosidase, alkaline phosphatase), radioactive isotopes (like ³²P or ¹²⁵I), biotin, digoxygenin, colloidal metals, chemi- or bioluminescent compounds (like dioxetanes, luminol or acridiniums). Labeling procedures, like covalent coupling of enzymes or biotinyl groups, iodinations, phosphorylations, biotinylations, etc. are well known in the art.

Detection methods comprise, but are not limited to, autoradiography, fluorescence microscopy, direct and indirect enzymatic reactions, etc. Commonly used detection assays comprise radioisotopic or non-radioisotopic methods. These comprise, inter alia, Westernblotting, overlay-assays, RIA (Radioimmuno Assay) and IRMA (Immune Radioimmunometric Assay), EIA (Enzyme Immuno Assay), ELISA (Enzyme Linked Immuno Sorbent Assay), FIA (Fluorescent Immuno Assay), and CLIA (Chemioluminescent Immune Assay).

Furthermore, another inventive use of the antibodies of the present invention is the use in a method for identifying a patient having or being at risk of developing a disease associated with human β1-adrenocepeptor. Accordingly, the antibody that is obtainable from the host cell with the deposit number DSM ACC3121 can be used in a method for identifying a patient having or being at risk of developing a disease associated with human β1-adrenocepeptor. The antibody that is obtainable from the host cell with the deposit number DSM ACC3174 can also be used in a method for identifying a patient having or being at risk of developing a disease associated with human β1-adrenocepeptor. The antibody that is obtainable from the host cell with the deposit number DSM ACC3175 can also be used in a method for identifying a patient having or being at risk of developing a disease associated with human β1-adrenocepeptor. The antibody that is obtainable from the host cell with the deposit number DSM ACC3176 can also be used in a method for identifying a patient having or being at risk of developing a disease associated with human β1-adrenocepeptor. The antibody that is obtainable from the host cell with the deposit number DSM ACC3177 can also be used in a method for identifying a patient having or being at risk of developing a disease associated with human β1-adrenocepeptor.

The above recited diseases associated with human β1-adrenoceptor comprise, but are not limited to heart diseases, comprising idiopathic dilated cardiomyopathy (DCM), ischaemic cardiomyopathy (ICM), infectious and non-infectious heart disease, ischemic and non-ischemic heart disease, inflammatory heart disease and myocarditis, cardiac dilatation, idiopathic cardio-myopathy, immune-cardiomyopathy, heart failure, and any cardiac arrhythmia including ventricular, Chagas disease and supraventricular premature capture beats.

In the context of the present invention, the disease associated with human β1-adrenoceptor refers to idiopathic dilated cardiomyopathy (DCM). Furthermore, in the context of the present invention, the disease associated with human β1-adrenoceptor refers to ischaemic cardiomyopathy (ICM).

Accordingly, the present invention provides a method for identifying a patient having or being at risk of developing a disease associated with human β₁-adrenoreceptor, comprising the steps of:
(a) contacting a human β₁-adrenoreceptor with a biological sample of said patient, thereby allowing molecule(s) or compound(s) contained in said biological sample to bind to the human β₁-adrenoreceptor;
(b) contacting the human β₁-adrenoreceptor of (a) with the antibody/binding compound of the present invention, thereby allowing said antibody/binding compound to bind to human β₁-adrenoreceptor which is not bound by molecule(s) or compound(s) contained in said biological sample of (a);
(c) contacting a human β₁-adrenoreceptor which was not contacted with said biological sample of (a) with the antibody/binding compound of the present invention, thereby allowing said antibody/binding compound to bind to said human β₁-adrenoreceptor which was not contacted with said biological sample of (a);
(d) measuring
   (i) a binding signal between the human β₁-adrenoreceptor and the antibody/binding molecule of step (b), and
   (ii) a binding signal between the human β₁-adrenoreceptor and the antibody/binding molecule of step (c); and
(e) comparing the binding signal measured in (d)(i) with that of (d)(ii),
   wherein a binding signal measured in (d)(i) which is at least 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% lower than that measured in (d)(ii) indicates that said patient has or is at risk of developing said disease.

The invention provides a method for identifying a patient having or being at risk of developing a disease associated with human β₁-adrenoreceptor, comprising the steps of:
(a) contacting a human β₁-adrenoreceptor with a biological sample of said patient, thereby allowing molecule(s) or compound(s) contained in said biological sample to bind to the human β₁-adrenoreceptor;
(b) contacting the human β₁-adrenoreceptor of (a) with the antibody or derivative thereof that is obtainable from the host cell, for example a hybridoma, with the deposit number DSM ACC3121, thereby allowing said antibody or derivative thereof to bind to human β₁-adrenoreceptor which is not bound by molecule(s) or compound(s) contained in said biological sample of (a);
(c) contacting a human β₁-adrenoreceptor which was not contacted with said biological sample of (a) with the antibody or derivative thereof that is obtainable from the host cell, for example a hybridoma, with the deposit number DSM ACC3121, thereby allowing said antibody or derivative thereof to bind to said human β₁-adrenoreceptor which was not contacted with said biological sample of (a);
(d) measuring
   (i) a binding signal between the human β₁-adrenoreceptor and the antibody or derivative thereof of step (b), and
   (ii) a binding signal between the human β₁-adrenoreceptor and the antibody or derivative thereof of step (c); and
(e) comparing the binding signal measured in (d)(i) with that of (d)(ii),
   wherein a binding signal measured in (d)(i) which is at least 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% lower than that measured in (d)(ii) indicates that said patient has or is at risk of developing said disease.

Furthermore, the invention provides a method for identifying a patient having or being at risk of developing a disease associated with human β₁-adrenoreceptor, comprising the steps of:
(a) contacting a human β₁-adrenoreceptor with a biological sample of said patient, thereby allowing molecule(s) or compound(s) contained in said biological sample to bind to the human β₁-adrenoreceptor;
(b) contacting the human β₁-adrenoreceptor of (a) with the antibody or derivative thereof that is obtainable from the host cell, for example a hybridoma, with the deposit number DSM ACC3174, thereby allowing said antibody or derivative thereof to bind to human β₁-adrenoreceptor which is not bound by molecule(s) or compound(s) contained in said biological sample of (a);
(c) contacting a human β₁-adrenoreceptor which was not contacted with said biological sample of (a) with the antibody or fragment thereof that is obtainable from the host cell, for example a hybridoma, with the deposit number DSM ACC3174, thereby allowing said antibody or derivative thereof to bind to said human β₁-adrenoreceptor which was not contacted with said biological sample of (a);
(d) measuring
   (i) a binding signal between the human β₁-adrenoreceptor and the antibody or derivative thereof of step (b), and
   (ii) a binding signal between the human β₁-adrenoreceptor and the antibody or derivative thereof of step (c); and
(e) comparing the binding signal measured in (d)(i) with that of (d)(ii),
   wherein a binding signal measured in (d)(i) which is at least 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% lower than that measured in (d)(ii) indicates that said patient has or is at risk of developing said disease.

The invention also relates to a method for identifying a patient having or being at risk of developing a disease associated with human β₁-adrenoreceptor, comprising the steps of:
(a) contacting a human β₁-adrenoreceptor with a biological sample of said patient, thereby allowing molecule(s) or compound(s) contained in said biological sample to bind to the human β₁-adrenoreceptor;
(b) contacting the human β₁-adrenoreceptor of (a) with the antibody or derivative thereof that is obtainable from the host cell, for example a hybridoma, with the deposit number DSM ACC3175, thereby allowing said binding compound to bind to human β₁-adrenoreceptor which is not bound by molecule(s) or compound(s) contained in said biological sample of (a);
(c) contacting a human β₁-adrenoreceptor which was not contacted with said biological sample of (a) with the antibody or derivative thereof that is obtainable from the host cell, for example a hybridoma, with the deposit number DSM ACC3175, thereby allowing said binding compound to bind to said human β₁-adrenoreceptor which was not contacted with said biological sample of (a);
(d) measuring
   (i) a binding signal between the human β₁-adrenoreceptor and the antibody or derivative thereof of step (b), and
   (ii) a binding signal between the human β₁-adrenoreceptor and the antibody or derivative thereof of step (c); and
(e) comparing the binding signal measured in (d)(i) with that of (d)(ii),
   wherein a binding signal measured in (d)(i) which is at least 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% lower than that measured in (d)(ii) indicates that said patient has or is at risk of developing said disease.

The invention also relates to a method for identifying a patient having or being at risk of developing a disease associated with human β₁-adrenoreceptor, comprising the steps of:
(a) contacting a human β₁-adrenoreceptor with a biological sample of said patient, thereby allowing molecule(s) or compound(s) contained in said biological sample to bind to the human β₁-adrenoreceptor;
(b) contacting the human β₁-adrenoreceptor of (a) with the antibody or derivative thereof that is obtainable from the host cell, for example a hybridoma, with the deposit number DSM ACC3176, thereby allowing said binding compound to bind to human β1-adrenoreceptor which is not bound by molecule(s) or compound(s) contained in said biological sample of (a);
(c) contacting a human β₁-adrenoreceptor which was not contacted with said biological sample of (a) with the antibody or derivative thereof that is obtainable from the host cell, for example a hybridoma, with the deposit number DSM ACC3176, thereby allowing said antibody or derivative thereof to bind to said human β₁-adrenoreceptor which was not contacted with said biological sample of (a);
(d) measuring
   (i) a binding signal between the human β₁-adrenoreceptor and the antibody or derivative thereof of step (b), and
   (ii) a binding signal between the human β₁-adrenoreceptor and the antibody or derivative thereof of step (c); and
(e) comparing the binding signal measured in (d)(i) with that of (d)(ii),
   wherein a binding signal measured in (d)(i) which is at least 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% lower than that measured in (d)(ii) indicates that said patient has or is at risk of developing said disease.

The invention provides a method for identifying a patient having or being at risk of developing a disease associated with human β₁-adrenoreceptor, comprising the steps of:
(a) contacting a human β₁-adrenoreceptor with a biological sample of said patient, thereby allowing molecule(s) or compound(s) contained in said biological sample to bind to the human β₁-adrenoreceptor;
(b) contacting the human β₁-adrenoreceptor of (a) with the antibody or derivative thereof that is obtainable from the host cell, for example a hybridoma, with the deposit number DSM ACC3177, thereby allowing said antibody or derivative thereof to bind to human β₁-adrenoreceptor which is not bound by molecule(s) or compound(s) contained in said biological sample of (a);
(c) contacting a human β₁-adrenoreceptor which was not contacted with said biological sample of (a) with the antibody or derivative thereof that is obtainable from the host cell, for example a hybridoma, with the deposit number DSM ACC3177, thereby allowing said antibody or derivative thereof to bind to said human β₁-adrenoreceptor which was not contacted with said biological sample of (a);
(d) measuring
   (i) a binding signal between the human β₁-adrenoreceptor and the antibody or derivative thereof of step (b), and
   (ii) a binding signal between the human β₁-adrenoreceptor and the antibody or derivative thereof of step (c); and
(e) comparing the binding signal measured in (d)(i) with that of (d)(ii),
   wherein a binding signal measured in (d)(i) which is at least 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% lower than that measured in (d)(ii) indicates that said patient has or is at risk of developing said disease.

In the context of the present invention, a binding signal is measured in (d)(i) which is at least 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% lower than that measured in (d)(ii) indicates that said patient has or is at risk of developing said disease. In particular, a binding signal measured in (d)(i) which is at least 65% lower than that measured in (d)(ii) identifies the tested patient as having or being at risk of developing a disease associated with human β1-adrenoreceptor.

In one further assay for identifying a patient having or being at risk of developing a disease associated with human β₁-adrenoreceptor the validation of the factor (K) and assay cut-off value can be determined as shown in the appended Examples in sections 5.1.1 to 5.1.3. In this assay the competitive efficacy of samples, NC (for example serum from healthy volunteers (control samples)) and PC (for example serum from healthy volunteers spiked with anti-β1-AR rat 13F6 antibody that is obtainable from the host cell, for example a hybridoma, with the deposit number DSM ACC3174) respectively, was calculated as percentage inhibition of the antibody 23-6-7, that is obtainable from the host cell, for example a hybridoma, with the deposit number DSM ACC3121, binding. To this end, each measured binding signal (optical density (OD) value) was divided by, for example, the value measured by the antibody 23-6-7, multiplied by 100, and the resulting values were subtracted from 100.

No reduction in OD value of for example the 23-6-7 mouse antibody resulted in 0% inhibition, whereas complete OD value reduction corresponds to 100% inhibition. Accordingly, within the context of the present invention the factor K can be determined on the analysis of sera from healthy subjects as control samples (NC) that do not suffer from a disease associated with human β₁-adrenoreceptor by using the following equations (1), (2) and (3):

Inhibition %_{screening cut-off}=mean Inhibition %_{row data}(control samples)+2×Standard Deviation (SD) (1)

Kᵢ=(Inhibition %_{screening cut-off i}−mean Inhibition %_{NC i})/mean Inhibition %_{PC I} (2)

K=(K₁+K₂+K₃)/3 (3)

By using equations (1), (2) and (3) in one further assay the factor (K)=0.143 can be obtained.

Kᵢ (i=1 to 3) can be determined on three plates with for example 20 blank individual samples. For all further plates “i” the following cut-off formula (4) can be applied:

Inhibition %_{cut-off i}=mean Inhibition %_{NC i}+K(0.143)×mean Inhibition % (4)

This way of Inhibition %_{cut-off} calculation can avoid the necessity to analyze a high number of individual blank samples on each plate. In order to adjust the Inhibition %_{row} data (sample) from different plates, the respective Inhibition %_{cut-off} has to be considered.

Inhibition %=mean Inhibition %_{row data}(sample)−Inhibition %_{cut-off} (5)

As illustrated in FIG. 11, in the method for identifying a patient having or being at risk of developing a disease associated with human β1-adrenoceptor, a competitive assay approach was developed: human anti-β1-AR (auto-) antibodies compete with mouse monoclonal antibodies, such as for example antibody 23-6-7 that is obtainable from a host cell with the deposit number DSM ACC3121, for the binding to cellular β1-ARs. Therefore, the binding signal between the human β1-AR and the antibody of the present invention was measured once in the presence of a biological sample containing, for example auto anti-β1 AR antibodies that bind to the second extracellular loop of the β1-AR. As control sample, the binding signal between the human β1-AR and the antibody of the present invention was measured in the absence of a biological sample containing, for example auto anti-β1 AR antibodies that bind to the second extracellular loop of the β1-AR. As explained above, no reduction in measured binding signal of the antibodies of the present invention presents 0% inhibition, whereas complete reduction (no measurable signal) presents 100% inhibition. The inhibition cut-off value as indicated in the above equations lies in the context of the present invention between 40% and 75%. Accordingly, the inhibition cut-off value in the context of the present invention lies between 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74% and 75%.

The diseases associated with human β1-adrenoceptor to be tested in the method of the present invention comprise, but are not limited to heart diseases, comprising idiopathic dilated cardiomyopathy (DCM), ischaemic cardiomyopathy (ICM), infectious and non-infectious heart disease, ischemic and non-ischemic heart disease, inflammatory heart disease and myocarditis, cardiac dilatation, idiopathic cardio-myopathy, immune-cardiomyopathy, heart failure, and any cardiac arrhythmia including ventricular, Chagas disease and supraventricular premature capture beats.

In the context of the method of the present invention, the human β1-adrenoceptor (β1-AR) is immobilized on a solid phase prior to contacting with a biological sample or the binding compound of the present invention.

Within the context of the method of the invention, the human β1-adrenoceptor (β1-AR) is immobilized on a solid phase on a surface after contacting with a biological sample or the binding compound/antibody of the present invention.

Receptors, preferably the human β1-adrenoreceptor (β1-AR) as used herein, can be immobilized on the solid phase in various ways. The appropriate methods depend on various factors, such as e.g., the type of receptor or the material of the solid phase. An immobilization can take place covalently or by adsorption. According to a preferred embodiment of the method of the present invention (as shown in the appended Examples), the receptor is a human β1-adrenoceptor which is expressed in SF9 cells and fixed on the solid phase (preferably on poly-L-Lysine coated culture plates). For the immobilization of immobilization of receptors which are proteins, methods are described in which the receptors are immobilized directly on a solid phase by means of passive adsorption. Normally, an appropriate solid phase consists of a polymer plastic material (e.g. p polystyrene, polyvinyl, latex) and e.g. in form of microtitre plates or multi-well plates, membranes or spheric “beads” (cross-linked polymers in particle form) are used for this purpose (Lowman, Annu Rev. Biophys. Biomol. Struct. 26 (1997), 401-24).

Furthermore, in the context with the method according to the invention, the material of the solid phase is selected from the group consisting of poly-L-Lysin, poly-L-Lysin precoated, sepharose, latex, glass, polystyrene, polyvinyl, nitrocellulose and silicon.

Further preferred, the solid phase in the method according to the invention is a membrane, a bead, a chip or a (culture) plate. Examples of the plates mentioned are microtitre plates or multi-well plates. Preferably, these have 6, 12, 24, 48, 96, 128, 356, 1024 or more wells. In Example 4 of the present invention, a method is described wherein 96 well plates are used.

Furthermore, in the context of the method for identifying a patient having or being at risk of developing a disease associated with human β₁-adrenoreceptor as described above, the detection of a binding signal between the human β1-adrenoreceptor or a fragment of this receptor with the first binding molecule in step (a), the biological sample is contacted with the binding compound described herein binding to the second extracellular loop of the human β1-adrenoreceptor, which is accessible after binding of the first binding molecule with the human β1-adrenoreceptor. This preferred embodiment relates, for example, to methods taking advantage of the mechanistic principle of the ELISA. This principle is generally known to the skilled person and is described among others, in Stryer, Biochemie, Spektrum Akademischer Verlag, 1996. Furthermore, a corresponding method is described in the appended Example 5.

Furthermore, in the context of the method described herein, the antibody as described herein is labelled. Moreover, it is preferred that the labelling of the binding molecule described herein comprises a system emitting signal. An example of such a system emitting a signal is the above described labelling with radioisotopes. Likewise, fluorescent labelling of the binding compounds as described herein results in the labelling with a system emitting a signal according to the invention, wherein the signal is the emission of a fluorescence signal after appropriate stimulation of the dye. According to the invention described herein, further preferred, the system emitting a signal comprises an enzyme emitting a signal. Examples of such enzymes comprise alkali phospatases, peroxidisases, β-galactosidase, glucoamylase and urease. Appropriate examples and the use of necessary substrates for the detection by means of enzymatic reactions are known to the skilled person, amongst others from the package leaflet of commercially available detection kits. Such commercially available kits often contain second molecule(s) or compound(s) which recognize the binding compound(s) (antibody(ies)) of specific species, e.g., anti-mouse, and to which enzymes emitting signals are coupled. Thus, corresponding antibodies are examples of the second molecule(s) or compound(s), which recognize a specific labelling of the binding compound(s) (antibody (ies)) as described herein, that is its Fc part.

In the context of the method as described herein, the second molecule(s) or compound(s) is (are) selected from the group consisting of peptides, polypeptides, low-molecular substances, antibodies or fragments or derivates thereof.

The term “peptide(s)” usually refers to amino acid chains with up to 30 amino acids. The term “polypeptide(s)” refers to peptides which usually comprise more than 30 amino acids and includes proteins. The term “low-molecular substances” or small molecule(s) refers to molecules which are of low molecular complexity having a molecular mass between 50 and 3000 g/mol, more often, however, between 75 and 2000 g/mol and mostly in the range between 100 and 1000 g/mol. Low-molecular substances can be of organic or inorganic nature.

The present invention also relates to a diagnostic kit for the detection of molecule(s) or compound(s) comprising at least the binding compound(s) of the present invention, at least the host cell of the present invention or at least the diagnostic agent/diagnostic molecule of the present invention. Advantageously, the kit of the present invention further comprises, optionally (a) buffer(s), storage solutions and/or remaining reagents or materials required for the conduct of medical, scientific or diagnostic assays and purposes. Furthermore, parts of the kit of the invention can be packaged individually in vials or bottles or in combination in containers or multicontainer units.

Accordingly, in the context of the present invention, the (diagnostic) kit refers to a kit for the detection of auto anti-β1-adrenergic antibody (antibodies), wherein the kit comprises at least the antibody that is produced by/obtainable from the host cell, for example a hybridoma, with the deposit number DSM ACC3121. The invention also relates to a kit for the detection of auto anti-β1-adrenergic antibody (antibodies), wherein the kit comprises at least the antibody that is produced by/obtainable from the host cell, for example a hybridoma, with the deposit number DSM ACC3174. The invention also relates to a kit for the detection of auto anti-β1-adrenergic antibody (antibodies), wherein the kit comprises at least the antibody that is produced by/obtainable from the host cell, for example a hybridoma, with the deposit number DSM ACC3175. The invention also relates to a kit for the detection of auto anti-β1-adrenergic antibody (antibodies), wherein the kit comprises at least the antibody that is produced by/obtainable from the host cell, for example a hybridoma, with the deposit number DSM ACC3176. The invention also relates to a kit for the detection of auto anti-β1-adrenergic antibody (antibodies), wherein the kit comprises at least the antibody that is produced by/obtainable from the host cell, for example a hybridoma, with the deposit number DSM ACC3177.

The kit of the present invention may be advantageously used, inter alia, for carrying out the method of the invention and could be employed in a variety of applications referred herein, e.g., as diagnostic kits, as research tools or medical tools. Additionally, the kit of the invention may contain means for detection suitable for scientific, medical and/or diagnostic purposes. The manufacture of the kits follows preferably standard procedures which are known to the person skilled in the art.

### EXAMPLES

The Figures show

FIG. 1: ELISA binding assay using the 26-meric peptide (His-Trp-Trp-Arg-Ala-Glu-Ser-Asp-Glu-Ala-Arg-Arg-Cys-Tyr-Asn-Asp-Pro-Lys-Cys-Cys-Asp-Phe-Val-Thr-Asn-Arg (SEQ ID NO:17)) Percentage of patients suffering from idiopathic dilated cardiomyopathy (DCM patients) considered anti-β1-AR positive and healthy volunteers (control patients), when using an ELISA binding assay by using the 26-meric peptide (His-Trp-Trp-Arg-Ala-Glu-Ser-Asp-Glu-Ala-Arg-Arg-Cys-Tyr-Asn-Asp-Pro-Lys-Cys-Cys-Asp-Phe-Val-Thr-Asn-Arg (SEQ ID NO:17)) coated onto plastic surface of microtiter plates.

FIG. 2: ELISA-based determination of the affinity of different clones of monoclonal murine antibody to second extracellular domain of the human β1-adrenoceptor (β1-ECII AR) Increasing concentrations of various monoclonal antibodies clones (ranging from 0.00017 to 133 nM) were incubated onto SF9 cells, overexpressing the human β1-adrenoreceptor (β1-AR) after bacoluviral infection. Each monoclonal antibody was purified from hybridoma supernatant by Protein G chromatography. Concentration was determined by BCA protein content analysis and purity was analysed by Coomassie stain. Means with SD derivation of duplicate determinations of the OD value from one representative experiment are shown.

FIG. 3: Overview on the concentration with half maximal efficacy response (EC50)-values of the five mouse monoclonal antibodiesproduced by the hybridoma clones 23-6-7, 47-12-9, 50-1-5, 55-3-10 and 28-2-7 binding to human β1-AR. The mean concentration with half maximal efficacy response (EC50) values were determined from four independent experiments. The mean EC50 value of hybridoma clone 23-6-7 was significantly lower (p<0.05) than those of hybridoma clones 47-12-9, 50-1-5, 55-3-10 and 28-2-7, as determined by comparing the respective pEC50 values (log₁₀(EC50)) by analysis of variance (ANOVA) followed by post-hoc LSD.

FIG. 4: Binding characteristics of various antibodies obtained from mouse, rat and goat respectively, to human β1-AR, which were overexpressed in SF9 cells (in presence or absence of 0.1% TWEEN 20). Increasing concentrations of various antibodies (ranging from 0.001 to 100 nM) were incubated on SF9 cells, which overexpressed human β1-AR after bacoluviral infection. Means of duplicate determinations of the OD ratio with standard deviation are plotted over antibody concentration (logarithmic scale).

FIG. 5: Comparison of the ELISA-based determination of the affinity of various antibodies to anti-β1-AR ECII in presence or absence of TWEEN 20. Increasing concentrations of various antibodies (ranging from 0.001 to 100 nM) were incubated with/without TWEEN 20 on SF9 cells, which overexpressed the human β1-AR after bacoluviral infection. Means of duplicate determinations of the OD ratio with standard deviation are plotted over antibody concentration (logarithmic scale).

FIG. 6: Competition of the monoclonal murine (23-6-7) antibody binding to the second β1-AR by cyclo (Ala-Asp-Glu-Ala-Arg-Arg-Cys-Tyr-Asn-Asp-Pro-Lys-Cys-Ser-Asp-Phe-Val-Gln) referring to SEQ ID NO:16. Various concentrations of the cyclo peptide Ala-Asp-Glu-Ala-Arg-Arg-Cys-Tyr-Asn-Asp-Pro-Lys-Cys-Ser-Asp-Phe-Val-Gln (as depicted in SEQ ID NO:16), ranging from 0.0001 μM-10 μM, were co-incubated with the murine monoclonal anti-β1-AR-ECII antibody as produced by the hybridoma deposited under the accession number DSM ACC3121 (23-6-7) on SF9 cells, overexpressing the human β1-AR by baculovirus infection. Means with S.E.M. of 4 independent measurements are plotted.

FIG. 7: Inhibition values above cut-off of sera taken from individual idiopathic dilated cardiomyopathy (DCM) patients. Values are calculated as the inhibition of a serum sample on the ratio of the signals elicited by a monoclonal mouse anti-β-1AR-ECII antibody (23-6-7) in cells expressing the human β1-AR vs. control cells (not expressing the human β1-AR) Inhibition in % is shown, lowering by the 95% confidence interval and exceeding the 65% cut-off value. Results from three independent experiments done, each with duplicates, are shown. The bars indicate means with S.E.M.

FIG. 8: Inhibition values above cut-off of sera taken from healthy volunteers. Values are calculated as the inhibition of a serum sample on the ratio of the signals elicited by a monoclonal mouse anti-β1-AR-ECII antibody (23-6-7) in cells expressing the human β1-AR vs. control cells (not expressing the human β1-AR) Inhibition in % is shown, lowering by the 95% confidence interval and exceeding the 65% cut-off value. Results from three independent experiments done, each with duplicates, are shown. The bars indicate means with S.E.M.

FIG. 9: Determination of the percentage of positive auto-anti-β1-AR antibodies binding to the second extracellular loop of the human β1-adrenoreceptor (β1-AR-ECII) in DCM patients or healthy controls. Derivation as positive was done by cut off determination of 65% inhibition, if also the 95% confidence interval of repeated measurements exceeds that value. Using this value, only in one person of the tested healthy control group (43 persons) auto-anti-β1-AR antibodies could be determined, whereas in 22 of 82 patients suffering from DCM auto-anti-β1-AR antibodies could be determined.

FIG. 10: Principle of the ELISA measurement of human anti-β1-AR (human β1-adrenoreceptor) antibodies via competition of the monoclonal mouse anti-β1-AR-ECII antibody 23-6-7. The ELISA mimics the in vivo auto-antibody binding characteristics to β1-ARs using a microstate plate format. In order to avoid cross-binding of other human antibodies to various cellular membrane proteins, a competitive approach was developed: human anti-β1-AR (auto-) antibodies compete with the mouse monoclonal antibodies, such as for example antibody 23-6-7 that is obtainable from a host cell with the deposit number DSM ACC3121, for the binding to cellular β1-ARs.

FIG. 11: Binding affinity of the mouse monoclonal antibody that is obtainable from the host cell/hybridoma with the deposit number DSM ACC3121 Binding affinity of the mouse monoclonal anti-β-1AR-ECII antibody 23-6-7, that is obtained from the host cell/hybridoma with the deposit number DSM ACC3121, to fully recombinant human β1-AR, overexpressed on SF9 cells after baculoviral infection. Means with S.E.M. of at least 4 independent measurements are plotted. Functionally relevant human anti-β1-AR auto-antibodies from patient sera are characterized by their capacity to bind to the same or overlapping epitopes and displace the test binding molecule/antibody and therefore reduce the immunological or biological signal like an ELISA signal that can be measured for example by using a Peroxidase (POD) based emitting system.

FIG. 12: Measurement of cAMP levels by Epac-FRET in human embryonic kidney HEK293 cells stably expressing human β1-ARs. Representative FRET ratio traces of independent experiments are presented (% corresponds to the relative change in YFP/CFP intensity ratio) by using human embryonic kidney HEK293 cells stably expressing human β1-AR (as described in DE 10 2010 018 878 A1). The decrease in FRET reflects an increase in intracellular cAMP.

(A) None of the inactive control antibodies induced a significant cAMP response in living cells. The viability of the cell is proven by additional stimulation by isoproterenol (Iso) at a concentration of 2.5 mol/L at the end of the experiment, which elicits a full cAMP response.

(B) In contrast, addition of the mouse monoclonal anti-β-1AR-ECII antibody 23-6-7 (that is obtained from the host cell/hydbridoma with the deposit number DSM ACC3121) elicited a relevant signal, which corresponds to 38.2% of the maximum possible signal, as was induced by additional administration of isoproterenol (Iso) at the end of this experiment.

(C) The signal intensity and kinetics were comparable to those from DCM patient sera previously judged anti-β1-AR antibody positive.

FIG. 13: Competition of the binding of the mouse monoclonal anti-antibody 23-6-7 that is obtainable from the host cell/hybridoma with the deposit number DSM ACC3121 by polyclonal goat anti-β1-AR antibodies. Various concentrations (ranging from 0.0 to 1.400 nmol/L (nM)) of polyclonal goat antibodies were co-incubated with the mouse monoclonal anti-β-1AR-ECII antibody 23-6-7 that is obtainable from the host cell (hybridoma) with the deposit number DSM ACC3121 at a final concentration of 0.26 nM. Addition of 10% serum pool derived from healthy volunteers was compared to buffer control and resulted in a similar dose-dependent effect Inhibition was exerted by at least 10 nM goat antibodies. Means with S.E.M. of at least 4 independent measurements are plotted.

FIG. 14: Determination of the auto-anti-β1-AR antibodies binding to the second extracellular loop of the human β1-adrenoreceptor (β1-AR-ECII) in DCM patients or healthy controls (Figures (A) and (B)) and ICM (Figure (C)) patients.

(A) Overview of the β1-AR binding activity of DCM patients (n=167) and control subjects who did not report any known heart disease (n=110) using an ELISA with SF9 cells overexpressing β1-AR vs. control SF9 cells (negative for β1-AR). The binding activity was determined by measuring the competition with the monoclonal anti-β1-AR antibody 23-6-7. Means with S.E.M. of 3 independent measurements are plotted.

(B) Identical serum samples of DCM patients (n=167) and control subjects who did not report any known heart disease (n=110) were analysed against the 26-meric peptide of His-Trp-Trp-Arg-Ala-Glu-Ser-Asp-Glu-Ala-Arg-Arg-Cys-Tyr-Asn-Asp-Pro-Lys-Cys-Cys-Asp-Phe-Val-Thr-Asn-Arg (SEQ ID NO:17). Binding activity was calculated as a ratio (sample optical density (OD) to 26-mer/sample OD to control well). An anti-β1-AR antibody positive score was defined as a ratio of >1.5. Means with S.E.M. of 2 independent measurements are plotted.

(C) Overview of the β1-AR binding activity of ICM patients (n=156) and control subjects who did not report any known heart disease (n=110) using an ELISA with SF9 cells overexpressing β1-AR vs. control SF9 cells (negative for β1-AR). The binding activity was determined by measuring the competition with the monoclonal anti-β1-AR antibody 23-6-7 that is obtainable from the host cell/hybridoma with the deposit number DSM ACC3121. Means with S.E.M. of 3 independent measurements are plotted.

FIG. 15: Comparison of the inhibitory effect of unaltered sera and the respective antibody-depleted serum fractions Comparison of the inhibitory effect of unaltered sera and the respective antibody-depleted serum fractions. The 20 serum samples were tested positive with a mean inhibition value of 13.1%. In contrast, all protein G-treated samples were tested negative with a mean inhibition value below cut-off value.

The Examples illustrate the invention:

### Example 1

### Production of Antibodies which are Directed Against the Second Extracellular Loop of the Human β1-Adrenoreceptor (β1-AR-ECII)

1.1 Production and Purification of the Fusion Protein GST-β1-ECII Construct

DNA fragments encoding the second extracellular loop of the human β1-adrenoreceptor plus flanked transmembrane amino acids (amino acids 195-225; ECII). More precisely, the DNA fragments encoding the amino acids 197-222 (His-Trp-Trp-Arg-Ala-Glu-Ser-Asp-Glu-Ala-Arg-Arg-Cys-Tyr-Asn-Asp-Pro-Lys-Cys-Cys-Asp-Phe-Val-Thr-Asn-Arg of SEQ ID NO:17) of the second extracellular loop of the plus the amino acid 195(Lys), 196(Met), 223(Ala), 224(Tyr) and 225(Ala) of the flanked transmembrane region of the human β1 adrenoceptor (β1-AR) were amplified by polymerase chain reaction (PCR) with an upstream BamHI and a downstream EcoRI restriction site for subcloning. The PCR fragments were restricted, and inserted into the pGEX-1λT-vector (Pharmacia, Uppsala, Sweden) in frame with the 3′-end of the coding sequence of bacterial glutathione-S-transferase. The obtained GST-β1-AR-ECII fusion protein construct was controlled by sequencing before transformation of E. coli XL-1 blue cells (Stratagene, Heidelberg, Germany).

Expression of the GST-β1-AR-ECII fusion protein was induced at 30° C. with 1 mM isopropyl-1-thio-b-D-galacto-pyranoside (IPTG) for 3 h. Subsequently, the cells were harvested on ice, pelleted (4000×g, 4° C. for 10 min), resuspended in a 1/10 volume of ice-cold PBS (phosphate-buffered saline: 140 mM NaCl, 2.7 mM KCl, 10.1 mM Na₂HPO₄, 1.8 mM KH₂PO₄, pH 7.3), and lysed with a French Press (SLM Instruments, Rochester, N.Y., USA) at 12 000 psi in the presence of 20 μg/ml DNAse I (Sigma) and 2 mM Mg₂SO₄. After addition of 0.2 mM phenylmethyl sulfonyl fluoride (PMSF), 5 mM ethylenediaminetetraacetic acid (EDTA), and 1% Triton X-100, the lysate was centrifuged (10000×g, 4° C. for 15 min) and the soluble protein fraction was adsorbed to a glutathione-Sepharose 4B column (Pharmacia, Uppsala, Sweden). After washing with PBS, bound proteins were eluted with 10 mM reduced glutathione in 50 mM Tris-HCl, pH 8.0. The purity of the eluates was controlled by sodium dodecyl sulfate-polyacrylamide gel electrophoresis (SDS-PAGE) and Coomassie blue staining. All the obtained products were essentially pure (80-90%); the only contaminant detectable was a split-product of 29 kDa, corresponding to bacterial glutathione-S-transferase. The yield of the purified fusion proteins varied from 2.5 mg to 15 mg per liter of induced bacterial culture (Jahns, Eur J Pharmacol 316 (1996), 111-121).

1.2 Production of Monoclonal Murine Antibodies which are Directed Against the Second Extracellular Loop of the Human β1-Adrenoreceptor (β1-AR-ECII)

1.2.1 Immunization

Eight week old BALB/c female mice were immunized subcutaneously over a period of 39 days with GST fusion protein linked with a 31-meric peptide (GST-β1-AR-ECII) as described above under item 1.1. The mice were immunized three times, every 2 weeks with 50 μg/rat of GST-β1-AR-ECII fusion protein with Freund's Adjuvant Complete plus Incomplete.

First immunisation was conducted with GST-β1-AR-ECII (50 μg) dissolved in 250 μl PBS, 200 μl Freund's Adjuvant Incomplete (Sigma-Aldrich®) and 50 μl Freund's Adjuvant Complete (Sigma-Aldrich®). Second and third immunisation were conducted with GST-β1-AR-ECII (50 μg) dissolved in 250 μl PBS and 250 μl Freund's Adjuvant Incomplete (Sigma-Aldrich®). The total volume of 500 μl was distributed to various locations for subcutaneous injections. After 39 days, 11 days after the third immunization, splenocytes were isolated from the spleen and were fused with immortalized myeloma cells SP2/0 with a ratio of 4:1 using polyethylene glycol. Fused cells were incubated in HAT medium (hypoxanthine-aminopterin-thymidine medium) for 10 days. Parallel to two single cell cloning procedures the hybridoma culture supernatants were screened and selected by ELISA using GST fusion protein, linear 25-meric peptide (Ala-Arg-Ala-Glu-Ser-Asp-Glu-Ala-Arg-Arg-Cys-Tyr-Asn-Asp-Pro-Lys-Cys-Cys-Asp-Phe-Val-Thr-Asn-Arg-Gln of SEQ ID NO:18 or the 18-meric cyclopeptide (cyclo Ala-Asp-Glu-Ala-Arg-Arg-Cys-Tyr-Asn-Asp-Pro-Lys-Cys-Ser-Asp-Phe-Val-Gln with Cys-Cys of SEQ ID NO:16) as immobilized antigen. Five different hybridoma cell clones were derived from this hybridoma fusion approach, i.e., hybridoma cell clone 23-6-7, 28-2-7, 47-12-9, 50-1-5 and 55-4-10.

1.2.2 Purification from Antibody from Hybridoma Cell Culture Supernatant

Hybridoma cells were cultured in DMEM (with 4.5 g/L Glucose, Na-Pyruvate, 2×10⁻³ M L-Glutamine, 2×10⁻³ M non-essential amino acid, 5×10⁻⁵ M 2-Mercaptoethanol, 15% FCS, 100 mg/L Steptomycin, 250 μg/L Amphotericin) at 37° C. with 5% CO₂. Subsequently, the supernatants from the hybridoma cell culture clones were purified by Protein G affinity chromatography. Antibody containing supernatants from cell culture clones were purified by Protein G Sepharose 4 Fast Flow (Thermo Fisher, cat. 17-0618-05). Before sample loading on the column the supernatants were centrifuged 15 min by 14000 g at 4° C. and mixed with equal volume of 20 mM Na₂PO₄ and 1/20 volume Protein G Sepharose 4 Fast Flow. After 1 h incubation at 20° C. the mixtures were transferred to centrifuge columns (Thermo Scientific, cat. 89897). The columns were washed with 30× column volume of 20 mM Na₂PO₄. Antibodies were eluted with 100 mM Glycin, pH 2.7. Immediately after elution the pH was restored with 1 M Tris/HCl pH 9.0 to pH 7.5. Samples were dialysed against PBS over night at 4° C. Purity was controlled by Coomassie blue staining and the concentration was determined by measurement the optical density at 280 nm.

1.2.3 Depository of the Mouse Monoclonal Antibody 23-6-7

The hybridoma clone 23-6-7 expressing the mouse monoclonal antibody 23-6-7 that binds against the second extracellular loop of the human β1-adrenoreceptor (β1-AR-ECII) has been deposited by the Corimmun GmbH, Fraunhoferstr. 17, D-82152 Martinsried at the DSMZ-Deutsche Sammlung von Mikroorganismen und Zellkulturen GmbH, Inhoffenstr. 7B, D-38124 Braunschweig, Germany on Mar. 15, 2011. The deposit name and the DSM accession number for the hybridoma (23-6-7) is “b1ECII E3, 23-6-7 (anti-beta1-AR)” and “DSM ACC3121 (DSMZ ACC3121)”.

1.2.4 Depository of the Mouse Monoclonal Antibody 28-2-7

The hybridoma clone 28-2-7 expressing the mouse monoclonal antibody 28-2-7 that binds against the second extracellular loop of the human β1-adrenoreceptor (β1-AR-ECII) has been deposited by the Corimmun GmbH, Fraunhoferstr. 17, D-82152 Martinsried at the DSMZ-Deutsche Sammlung von Mikroorganismen und Zellkulturen GmbH, Inhoffenstr. 7B, D-38124 Braunschweig, Germany on May 16, 2012. The deposit name and the DSM accession number for the hybridoma (23-6-7) is “b1ECII, 28-2-7” and “DSM ACC3175 (DSMZ ACC3175)”.

1.2.5 Depository of the Mouse Monoclonal Antibody 47-12-9

The hybridoma clone 47-12-9 expressing the mouse monoclonal antibody 47-12-9 that bids against the second extracellular loop of the human β1-adrenoreceptor ECII) has been deposited by the Corimmun GmbH, Fraunhoferstr. 17, D-82152 Martinsried at the DSMZ-Deutsche Sammlung von Mikroorganismen und Zellkulturen GmbH, Inhoffenstr. 7B, D-38124 Braunschweig, Germany on May 16, 2012. The deposit name and the DSM accession number for the hybridoma (47-12-9) is “b1ECII, 47-12-9” and “DSM ACC3176 (DSMZ ACC3176)”.

1.2.6 Depository of the Mouse Monoclonal Antibody 50-1-5

The hybridoma clone 50-1-5 expressing the mouse monoclonal antibody 50-1-5 that binds against the second extracellular loop of the human β1-adrenoreceptor ECII) has been deposited by the Corimmun GmbH, Fraunhoferstr. 17, D-82152 Martinsried at the DSMZ-Deutsche Sammlung von Mikroorganismen und Zellkulturen GmbH, Inhoffenstr. 7B, D-38124 Braunschweig, Germany on May 16, 2012. The deposit name and the DSM accession number for the hybridoma (50-1-5) is “b1ECII, 50-1-5” and “DSM ACC3177 (DSMZ ACC3177)”.

1.3 Production of Monoclonal Rat and Goat Polyclonal Antibodies which are Directed Against the Second Extracellular Loop of the Human β1-Adrenoreceptor (β1-AR-ECII)

The rat monoclonal antibody clone 13F6 was produced according the same protocol as the one described above for mouse monoclonal antibodies (see items 1.2.1 and 1.2.2, supra). More precisely, the rat monoclonal antibody clone 13F6 was produced by In Vivo Biotech Services GmbH using the GST-β1-ECII fusion protein (see item 1.1, supra) as used for mouse monoclonal antibodies (see items 1.2.1 and 1.2.2, supra). The rat antibody was subsequently purified by Protein G affinity chromatography according to the manufacturer's instruction and dissolved in PBS.

The hybridoma (host cell) expressing the rat monoclonal antibody 13F6 that binds against the second extracellular loop of the human β1-adrenoreceptor (β1-AR-ECII) has been deposited by the Corimmun GmbH, Fraunhoferstr. 17, D-82152 Martinsried at the DSMZ-Deutsche Sammlung von Mikroorganismen and Zellkulturen GmbH, Inhoffenstr. 7B, D-38124 Braunschweig, Germany on May 16, 2012. The deposit name and the DSM accession number for the hybridoma cell (host cell) expressing the rat monoclonal antibody (clone) 13F6 is “13/F6” and “DSM ACC3174 (DSMZ ACC3174)”.

Goat polyclonal antibodies (Lot: 28498) were generated by Biogenes GmbH, Berlin. The immunisation of the goat was carried out by six boosts at day: 7, 14, 28, 70, 105, 133 by using the GST fusion protein (GSTβ1-AR-ECII) corresponding to the amino acids 197-222 of the second extracellular loop of the human β1-AR plus amino acids 195(L), 196(M), 223(A), 224(Y) and 225(A) of the flanked transmembrane region of the human β1 adrenoceptor (β1-AR) (see item 1.1, supra). At day 161 the antibody-containing serum was obtained and purified by affinity chromatography according to the manufacturer's instruction. Therefore the 25-meric peptide (Ala-Arg-Ala-Glu-Ser-Asp-Glu-Ala-Arg-Arg-Cys-Tyr-Asn-Asp-Pro-Lys-Cys-Cys-Asp-Phe-Val-Thr-Asn-Arg-Gln of SEQ ID NO:18) corresponding to amino acids 200-222 (Arg-Ala-Glu-Ser-Asp-Glu-Ala-Arg-Arg-Cys-Tyr-Asn-Asp-Pro-Lys-Cys-Cys-Asp-Phe-Val-Thr-Asn-Arg of SEQ ID NO:19) of the second extracellular loop of the human β1-AR plus the amino acids Ala at position 1 and Gln at position 25 was coupled to CNBr-activated Sepharose 4B (GE Healthcare, cat. 17-0430-01). The antibody was dissolved in Glycine-buffer, pH 7.5, 250 mM NaCl, 0.02% Thimerosal.

### Example 2

### Determination of the Coding Sequences of the Variable Regions of the Monoclonal Antibodies Against the Second Extracellular Loop of the Human β1-Adrenoreceptor (β1-ECII)

2.1 Determination of the coding sequences of the variable regions of the mouse monoclonal antibody 23-6-7 that is obtainable from the host cell (hybridoma) with the deposit number DSM ACC3121

The mRNA of the hybridoma cell (clone) “b1ECII E3, 23-6-7 (anti-beta1-AR)” (as deposited under DSM ACC3121) was isolated from 5×10⁶ cells using the Oligotex Direct mRNA kit (QIAGEN, Germany). The cDNA synthesis was performed using the SuperScript® III First-Strand Synthesis System (Invitrogen, USA). The amplification of variable region sequences by PCR was conducted according the protocol from Dübel, J Immunol Methods. 175 (1994), 89-95. Briefly, PCR was performed with 2 μl cDNA, 200 μM dNTP, 5% DMSO, 10 pmol primer each and 0.5 μl Herculase II Fusion (Agilent Technologies, USA) and 1× Herculase reaction buffer. The variable region sequence of the light chain variable region were amplified with the primer combination Bi8/Bi5 and the heavy chain sequence by using Bi3/Bi4 and Bi3d/Bi4 as amplification primers; for primer sequences see Table 2 below. As a positive control for cDNA quality primers (forward primer: 5′-GGCATCCTCACCCTGAAGTA-3′ (SEQ ID NO:20), reverse primer: 5′-GTCAGGCAGCTCGTAGCTCT-3′(SEQ ID NO:21)) for amplification of β-Actin were used. The negative control used water instead of cDNA. The amplification started with an initial denaturation at 95° C. for 2 min followed by 35 cycles of 94° C. for 1 min, 52° C. for 2 min, 72° C. for 1 min and a final extension of 72° C. for 5 min. PCR fragments were isolated from a 1.6% agarose gel (High Resolution agarose gels) and purified using the GFX PCR DNA and Gel Band Purification Kit (GE Healthcare, UK) according to the manufacturer's protocol. Purified PCR fragments were sequenced with primers named Bi5seq (5′-GGGAAGATGGATCCAGTTG-3′ (light chain; SEQ ID NO:27)) and Bi4seq (5′-CAGGGGCCAGTGGATAGA-3′ (heavy chain; SEQ ID NO:28)) and analyzed with NCBI IgBlast program (http://www.ncbi.nlm.nih.gov/igblast/).

2.2 Determination of the Coding Sequences of the Variable Regions of the Mouse Monoclonal Antibodies 28-2-7, 47-12-9 and 50-1-5 that are Obtainable from the Host Cell (Hybridoma) with the Deposit Numbers DSM ACC3175, DSM ACC 3176 and DSM ACC3177.

The mRNA of the hybridoma cells (clones) (i) “b1ECII, 28-2-7” as deposited under DSM ACC3175, (ii) “b1ECII, 47-12-9” as deposited under DSM ACC3176 and (iii) “b1ECII, 50-1-5” as deposited under DSM ACC3176 was isolated from 5×10⁶ cells using the Oligotex Direct mRNA kit (QIAGEN, Germany). The cDNA synthesis was performed using the SuperScript® III First-Strand Synthesis System (Invitrogen, USA). The amplification of variable region sequences by PCR was conducted according the protocol from Dübel, J Immunol Methods. 175(1994), 89-95. Briefly, PCR was performed with 0.5-1.0 μl cDNA, 200 μM dNTP, 2.5% DMSO, 10 pmol primer each and 0.5 μl Herculase II Fusion (Agilent Technologies, USA) and 1× Herculase reaction buffer. The variable region sequence of the light chain variable region were amplified with the primer combination Bi8/Bi5 and the heavy chain sequence by using Bi3/Bi4 and Bi3d/Bi4 as amplification primers; for primer sequences see Table 2 below. As a positive control for cDNA quality primers (forward primer: 5′-GGCATCCTCACCCTGAAGTA-3′ (SEQ ID NO:20), reverse primer: 5′-GTCAGGCAGCTCGTAGCTCT-3′(SEQ ID NO:21)) for amplification of β-Actin were used. The negative control used water instead of cDNA. The amplification started with an initial denaturation at 95° C. for 2 min followed by 35 cycles of 94° C. for 1 min, 52° C. for 2 min, 72° C. for 1 min and a final extension of 72° C. for 5 min. PCR fragments were isolated from a 1.6% agarose gel (High Resolution agarose gels) and purified using the GFX PCR DNA and Gel Band Purification Kit (GE Healthcare, UK) according to the manufacturer's protocol. Purified PCR fragments were sequenced with primers named Bi5seq (5′-GGGAAGATGGATCCAGTTG-3′ (light chain; SEQ ID NO:27)) and Bi4seq (5′-CAGGGGCCAGTGGATAGA-3′ (heavy chain; SEQ ID NO:28)) and analyzed with NCBI IgBlast program (http://www.ncbi.nlm.nih.gov/igblast/).

2.3 Determination of the Coding Sequences of the Variable Regions of the Rat Monoclonal Antibody 13F6 that is Obtainable from the Hybridoma (Host Cell) with the Deposit Number DSM ACC3174

The mRNA of hybridoma cell (clone) (i) “13F6” as deposited under DSM ACC3174 was isolated from 5×10⁶ cells using the Oligotex Direct mRNA kit (QIAGEN, Germany). The cDNA synthesis was performed using the SuperScript® III First-Strand Synthesis System (Invitrogen, USA). The amplification of variable region sequences by PCR was conducted according the protocol from Dübel, J Immunol Methods. 175 (1994), 89-95. Briefly, PCR was performed with 0.5-1.0 μl cDNA, 200 μM dNTP, 2.5% DMSO, 10 pmol primer each and 0.5 μl Herculase II Fusion (Agilent Technologies, USA) and 1× Herculase reaction buffer. The variable region sequence of the light chain variable region were amplified with the primer combination Bi7/Bi5 and the heavy chain sequence by using Bi3d/Bi4 as amplification primers; for primer sequences see Table 2 below. As a positive control for cDNA quality primers (forward primer: 5′-GGCATCCTCACCCTGAAGTA-3′ (SEQ ID NO:20), reverse primer: 5′-GTCAGGCAGCTCGTAGCTCT-3′(SEQ ID NO:21)) for amplification of β-Actin were used. The negative control used water instead of cDNA. The amplification started with an initial denaturation at 95° C. for 2 min followed by 35 cycles of 94° C. for 1 min, 52° C. for 2 min, 72° C. for 1 min and a final extension of 72° C. for 5 min. PCR fragments were isolated from a 1.6% agarose gel (High Resolution agarose gels) and purified using the GFX PCR DNA and Gel Band Purification Kit (GE Healthcare, UK) according to the manufacturer's protocol. Purified PCR fragments were sequenced with primers named Bi5seq (5′-GGGAAGATGGATCCAGTTG-3′ (light chain; SEQ ID NO:27)) and Bi4seq (5′-CAGGGGCCAGTGGATAGA-3′ (heavy chain; SEQ ID NO:28)) and analyzed with NCBI IgBlast program (http://www.ncbi.nlm.nih.gov/igblast/).

TABLE 2

| Primer | domain | 5′→3′ sequence |
|---|---|---|
| Bi7 | | GGTGATATC(A/T)TG(A/C)TGACCCAA(A/T)CTCCACTCTC |
| | | (SEQ ID NO: 29) |
| Bi8 | κ chain | GGTGATATCGT(G/T)CTCAC(C/T)CA(A/G)TCTCCAGCAAT |
| | variable | (SEQ ID NO: 22) |
| Bi5 | κ chain | GGGAAGATGGATCCAGTTGGTGCAGCATCAGC (SEQ ID |
| | constant | NO: 23) |
| Bi3 | heavy chain | GAGGTGAAGCTGCAGGAGTCAGGACCTAGCCTGGTG |
| | variable | (SEQ ID NO: 24) |
| Bi3d | heavy chain | AGGT(C/G)CAGCTGCAG(C/G)AGTC(A/T)GG (SEQ ID NO: 25) |
| | variable | |
| Bi4 | γ chain | CCAGGGGCCAGTGGATAGACAAGCTTGGGTGTCGTTTT |
| | constant | (SEQ ID NO: 26) |

### Example 4

### Heterologous Expression of the Human β1-Adrenoreceptor in Sf9 Insect Cells

The insect cells Sf9 cells (Spodoptera frugiperda, ATCC accession number CRL 1711) were grown in adhesion culture in Grace's Insect Medium (Invitrogen) supplemented with 10% fetal calf medium, 100 U/ml penicillin and 100 μg/ml streptomycin at 27° C. Cells were detached from culture flasks after 3-4 days of growth, when they had reached about 70-100% confluence. Afterwards, they were centrifuged (400×g, 5 min) at 20° C. and resuspended in cell culture medium (Grace's Insect Medium supplemented with 10% fetal calf medium, 100 U/ml penicillin and 100 μg/ml streptomycin). Suspended cells were infected with baculovirus (MOI 6) at 20° C., carrying the gene for the human β1-adrenoreceptor (β1-AR). A transgene-free baculovirus served as control. Cell suspension was directly seeded onto poly-L-lysine coated 96 well cell culture plates (Biocoat, #356516) at a density of 30,000 cells per well in a total of 200 μl culture medium (Grace's Insect Medium (Invitrogen) supplemented with 10% fetal calf medium, 100 U/ml penicillin and 100 μg/ml streptomycin). After 72 h incubation at a temperature of 27° C., 100 μl of the cell free culture supernatant was removed and 100 μl 2×PFA (Parafromaldehyde) fixation solution (2% PFA in the final solution in PBS) was added. Cells were incubated for 15 min at RT at constant shaking (Heidolph Titramax 1000, 450 rpm). Supernatants were removed subsequently and fixed cells were washed three times with PBS-T (PBS Dulbecco (Cat No. L1820, Biochrom AG)+0.1% TWEEN 20 (PBS-T)).

In order to provide the native and functionally active human β1-adrenoreceptor as binding epitope for auto anti-β1-adrenergic antibodies in a cell based (cellular) ELISA assay (see Example 5.1), SF9 cells were infected with baculovirus, carrying the gene for the human β1-AR. Direct measurement of patients' auto anti-β1-AR antibodies (auto anti-β1-AR antibody titers) was not possible, due to the strong background binding signal to cell-surface epitopes by the highly diversified human antibody pool. In order to circumvent this problem, a competition assay was performed, whereby a high affinity antibody against human β1-AR was used to generate a specific binding signal to the human β1-AR producing SF9 cells, which can be competed by specific anti-β1-AR auto-antibodies from human sera (FIG. 11).

4.1 Identification and Characterisation of the Monoclonal and Polyclonal Antibodies that Bind Against the Second Extracellular Domain of the β1-Adrenoreceptor (β1-AR)

A prerequisite for such a competitive approach, however, was the generation of an antibody with high specificity and affinity to human β1-AR. Different monoclonal mouse antibodies that bind to the second extracellular loop of the human β1-adrenic receptor (β1-adrenoreceptor) (β1-AR ECII) were produced by using a hybridoma cell-line approach (see Example 1.2). The binding characteristics of the five hybridoma cell clones 23-6-7, 28-2-7, 47-12-9, 50-1-5 and 55-3-10 to the (native) human β1-AR were analysed.

FIGS. 2 and 3 show a significantly better binding affinity of the hybridoma cell clone 23-6-7 compared to the other, similarly produced, antibodies (i.e., hybridoma cell clones 28-2-7, 47-12-9, 50-1-5 and 55-3-10). Hybridoma cell clone 23-6-7 provided highest binding affinity, K_{d}=0.43 nM (equal to EC50) in combination with low background level. These properties enhanced the competitive displacement with human auto-antibodies against β1-AR ECII significantly.

For further characterization of the antibody 23-6-7 (that is obtainable from the deposited hybridoma cell (clone) DSM ACC3121), the binding specificity to the second extracellular domain of the human β1-adrenoreceptor was tested. Therefore, various concentrations of the antibody 23-6-7 (that is obtainable from the deposited hybridoma cell (clone) DSM ACC3121) on recombinant β1-AR-overexpressing SF9 cells and initially measured its binding characteristics in the absence of any competitor. The results are shown in FIG. 12 which confirms the above experiments and illustrates a binding affinity of 0.43 nM for the antibody 23-6-7 (that is obtainable from the deposited hybridoma cell (clone) DSM ACC3121).

To determine the functionality of the 23-6-7 anti-β1-AR antibody clone, we investigated its ability to activate receptor-mediated intracellular cyclic adenosine monophosphate (cAMP) accumulation through sequential activation of Gₛ proteins and adenylyl cyclase (AC). One method to detect this increase in intracellular cAMP is to use fluorescence resonance energy transfer (FRET) between cyan fluorescence proteins (CFP) and yellow fluorescent proteins (YFP) fused to the cAMP-binding domain of Epac1 (Nikolaev. J Am Coll Cardiol 50 (2007), 423-43) The means and methods for the determination of an increase intracellular cAMP by using the resonance energy transfer (FRET) technique are described in DE 10 2010 018 878 A1. Addition of clone 23-6-7 clearly activated HEK 293 cells stably expressing human β1-AR, as determined by using this FRET assay (FIG. 12(B)), with slow kinetics as are typically exerted by anti-β1-AR auto-antibodies from DCM patients (FIG. 12(C)). In contrast, a negative control antibody was ineffective (FIG. 12(A)).

Accordingly, the antibody that is obtainable from the host cell (hybridoma) with the deposit number DSM ACC3121 represents in view of its high binding affinity to the β1-adrenoreceptor anantibody clone with which the competing assay could be reliably carried out.

Additionally, also rats and goats were immunized with a GST-β1-ECII fusion construct (see Example 1.1), resulting in the production of antibodies that bind against the second extracellular loop of the human β1-adrenoreceptor (β1-AR-ECII). In case of rat, the monoclonal antibody 13F6 was also produced by the hybridoma cell-line approach (see Example 1.3). Polyclonal goat antibody was purified by affinity chromatography with the β₁-AR-EC_{II} peptide (see Example 1.4). The comparison of the results obtained with mouse 23-6-7, rat monoclonal antibody and goat polyclonal antibody are shown in FIGS. 3 and 4.

In sum, as it is evident from FIGS. 3 and 4 of the present invention, the mouse monoclonal antibody as produced by the hybridoma cell clone 23-6-7 (as deposited under the accession number DSM ACC3121) binds with highest affinity to human β1-AR (as expressed in SF9 cells), followed by the rat monoclonal antibody 13F6 and the goat polyclonal antibody. The EC50 value of 0.41 nM of the mouse monoclonal antibody 23-6-7 is more than 10-times lower in comparison to the rat monoclonal antibody 13F6 and the goat polyclonal antibody. Moreover, an increase in signal intensity, respectively OD value, after adding 0.1% TWEEN to the assay incubation and washing buffer was measured. Removal of the detergent TWEEN 20 was, however, not sufficient for a competition measurement of the human auto-antibodies due to the very low signal to noise ratio.

In order to prove the binding specificity of the antibody 23-6-7 to the recombinant β1-AR overexpressed in SF9 cells, we added the 18-meric peptide (cyclo (Ala-Asp-Glu-Ala-Arg-Arg-Cys-Tyr-Asn-Asp-Pro-Lys-Cys-Ser-Asp-Phe-Val-Gln of SEQ ID NO:16) which corresponds to the core region of the second extracellular loop of the human β1 adrenoreceptor (β1-AR ECII-loop), to compete the antibody binding. The result is shown in FIG. 6 which shows an IC50 concentration in a low nanomolar range.

### Example 5

### Enzyme-Linked Immunoassays (ELISA)

5.1 Cellular Elisa Assay

The PFA fixed cells were blocked with 200 μl PBS-T (PBS Dulbecco (Cat No. L1820, Biochrom AG)+0.1% TWEEN 20) supplemented with 3% milk powder for 1 h at RT. Afterwards, the plates were washed three times with PBS-T. The mouse monoclonal anti β1-AR antibody as obtained from the hybridoma fusion approach, i.e., hybridoma cell clone 23-6-7 (see Example 1, supra) was added at a fixed concentration of sera 0.26 nM in the presence of 0.1% TWEEN 20 and 3% BSA (bovine serum albumin) and the binding was competed by addition of human sera (1:10 diluted) in the presence of 0.1% TWEEN 20 from healthy volunteers or from DCM patients, respectively. In a first series of experiments sera from 82 DCM patients and 43 sera were obtained from healthy volunteers (see Example 5.3) were investigated as shown and illustrated in FIGS. 7 to 9.

Positive control samples were provided by defined concentrations (760 nM) of the produced monoclonal rat antibody 13F6 that is obtainable from the hybridoma cell (clone) as deposited under DSM ACC3174 (see Example 1, supra) which were also used for competition. After incubation for 2 h at RT with constant shaking, the cells were washed three times with PBS-T and secondary antibody (Dianova, cat. 715-035-151) solution (diluted 1:5000 in PBS-T+3% milk powder) was added. Plates were incubated for 1 h at RT. After four further washing steps with PBS-T, peroxidase bound in the complex is visualized by 100 μl TMB (3,3′,5,5′-tetramethylbenzidine) substrate solution at 20° C. After stopping the enzymatic reaction with 100 μl of 1 M sulfuric acid, the intensity of the resulting colour was determined at 450 nm, and at a reference wavelength of 595 nm. The colour intensity was proportianally inverse to the amount of human anti-β1 receptor antibodies in the sample.

The optical density (OD) signal (human β1-AR Sf9 expressing cells) elicited by the mouse monoclonal antibody 23-6-7 (as deposited under the accession number DSM ACC3121) minus the respective OD background signal (control cells) was scored as 100% and the inhibitory capacity of each serum was determined in duplicates. The mean values of each serum with SEM of at least 3 independent experiments was calculated.

The differences between the groups were strongly significant (p<0.00005 for DCM vs. healthy control). Patients suffering from idiopathic dilated cardiomyopathy (DCM patients) had been investigated by echocardiography and are characterized by having an ejection fraction of less than 45%. Additionally, coronary heart disease had been excluded by invasive catheter investigation. Subjects with no known heart disease served as controls (healthy volunteers). The total number of tested patients suffering from idiopathic dilated cardiomyopathy (DCM patients) was 82 and the total number of healthy volunteers (not suffering from any known heart disease) was 43. Assay cut-off values were determined in order to classify auto-β1-adrenergic antibody positive (AR auto-antibody positive) and auto-β1-adrenergic antibody negative (AR auto-antibody negative) Inhibition of more than 65% was considered positive, if also the 95% confidence interval of repeated measurements exceeded that value.

Using this value, only one individual of the healthy control group (1/43) equal to 2.33% was considered as positive. In contrast 40.24% of DCM patients (33/82) were considered as anti β1-AR auto-antibody positive. To perform an overview of the inhibition capacity (%) of each DCM patient or healthy control, respectively, the results are plotted in histograms (FIG. 9).

5.1.1 Cell-Based β1-AR Competition Assay

Sf9 (Spodoptera frugiperda, ATCC accession number CRL 1711) cells were grown in adhesion culture according to standard cell culture protocols (for culture details see item Example 4, supra). Cells were detached from culture flasks after 3-4 days of growth, when they had reached about 70-100% confluence. Afterwards, they were centrifuged (400×g, 5 min) and resuspended in cell culture medium. Suspended cells were infected with baculovirus (MOI 6), carrying the gene for the human β1-AR. A transgene-free baculovirus served as control. Cell suspension was directly seeded on poly-L-lysine coated 96 well cell culture plates (Biocoat, #356516) at a density of 30,000 cells per well. After 72 h incubation, half of the cell culture supernatant (200 μl/well) was removed and 100 μl 2×PFA fixation solution (2% PFA in the final solution) was added. Cells were incubated for 15 min at RT at constant shaking Supernatants were removed subsequently and fixed cells were washed three times with PBS (PBS Dulbecco (Cat No. L1820, Biochrom AG)+0.1% TWEEN 20 (PBS-T). Optionally the microtiter plates were frozen at −80° C. for up to 6 months.

The PFA-fixed cells were blocked with 200 μl PBS-T+3% milk powder for 1 h at RT. Afterwards, the plates were washed three times with PBS-T. Mouse monoclonal anti β1-AR antibody 23-6-7 that is obtainable from the host cell with the deposit number DSM ACC3121 was added, then 23-6-7 binding was competed by addition of human sera from healthy volunteers or from DCM patients, respectively. Positive control samples were provided by defined concentrations of the monoclonal rat anti-β1-AR antibody 13F6 (that is obtainable from the host cell with the deposit number DSM ACC3174), which were also used for competition. After incubation for 2 h at RT with constant shaking, the cells were washed three times with PBS-T and secondary antibody solution (1:5000 in PBS-T+3% milk powder) was added. Plates were incubated for 1 h at RT. After a further washing step, 3× with PBS-T, peroxidase bound in the complex was visualized by TMB (3,3′,5,5′-tetramethylbenzidine) substrate solution. After stopping the enzymatic reaction with sulfuric acid, the intensity of the resulting colour was determined at 450 nm, and at a reference wavelength of 595 nm.

The competitive efficacy of human samples, NC (serum from healthy volunteers) and PC (serum from healthy volunteers spiked with anti-β1-AR rat 13F6 antibody) respectively, was calculated as percentage inhibition of the mouse antibody (23-6-7) binding. To this end, each OD value was divided by the mouse antibody (23-6-7) value, multiplied by 100, and the resulting values were subtracted from 100.

No reduction in OD value of the (23-6-7) mouse antibody resulted in 0% inhibition, whereas complete OD value reduction corresponded to 100% inhibition.

The assay validation was conducted for the determination of the factor (K) and assay cut-off value. In three independent experiments based on the analysis of sera from 20 healthy volunteers, the factor (K)=0.143 was obtained by using equation (1, 2, 3).

Inhibition %_{screening cut-off}=mean Inhibition %_{row data}(control samples)+2×SD (1)

Kᵢ=(Inhibition %_{screening cut-off i}−mean Inhibition %_{NC i})/mean Inhibition %_{PC I} (2)

K=(K₁+K₂+K₃)/3 (3)

Kᵢ(i=1 to 3) was determined on three plates with 20 blank individual samples For all further plates “i” the following cut-off formula (4) was applied:

Inhibition %_{cut-off i}=mean Inhibition %_{NC i}+K(0.143)×mean Inhibition %_{PCi} (4)

This way of Inhibition %_{cut-off} calculation avoided the necessity to analyze a high number of individual blank samples on each plate.
In order to adjust the Inhibition %_{row data} (sample) from different plates, the respective Inhibition %_{cut-off} has to be considered.

Inhibition %=mean Inhibition %_{row data}(sample)−Inhibition %_{cut-off} (5)

The hypothesis was that the binding of the monoclonal anti-β1-AR antibody 23-6-7 (that is obtainable from the hybridoma cell (clone) as deposited under DSM ACC3121) to β1-AR-overexpressing SF9 cells should be modified by co-incubation with serum from patients suffering from a disease associated with human β1-adrenorecptor (e.g., patients suffering from DCM; see schematic overview in FIG. 11). A competitive reduction of the 23-6-7 antibody that is obtainable from the host cell with the deposit number DSM ACC3121 binding should occur, depending on the presence of anti-beta1-AR antibodies in the respective sample. To clarify the impact of adding this human serum pool to the assay, the polyclonal goat anti-β1-AR antibodies were also added in control buffer in an identical assay approach. FIG. 13 shows high similarity of the competition curve for both conditions, regarding both, dose-dependency and maximum signal. In order to validate the assay, a negative control, consisting of a serum pool from healthy volunteers, was deemed to be necessary. Assay sensitivity was determined at 10 nM when using the polyclonal goat anti-β1-AR antibody for competition.

5.1.2 Validation of the β1-AR ELISA

To warrant inter-assay comparability, a negative control sample (NC), consisting of pooled human serum samples from healthy volunteers and a positive control (PC), consisting of a human serum pool spiked with the rat anti-β1-AR antibody 13F6 (that is obtainable from the hybridoma cell (clone) as deposited under DSM ACC3174) were measured on each microtiter plate. We used the monoclonal rat 13F6 antibody (that is obtainable from the hybridoma cell (clone) as deposited under DSM ACC3174) rather than the polyclonal goat anti-β1-AR antibody because of its reproducible availability.

In order to classify the inhibition (%) of the human serum samples, the plate specific Inhibition %_{cut-off} was considered. Responses varied between individual assays—therefore, cut-off values were modified accordingly. The use of the negative control plus a predetermined factor (K) to assess the cut-off value in each assay allowed to correct for changes of the non specific binding (NSB) over time. The additional use of the positive control in the cut-off formula allowed for an even better normalization, because only the OD value of the positive control allows an assessment of assay sensitivity.

Assay Cut-Off Point Value:

The cut-off value was determined statistically based on the level of non-specific background of the assay and the response of those matrix samples, above which a positive response was detected. In three independent experiments, serum samples from 20 healthy volunteers were examined. The mean+2.0×SD was calculated to determine the cut-off. In order to account for some smaller variation between individual assays, an adjusted cut-off value was calculated by multiplying with a specific normalization factor, determined from the pre-study validation data.

Sensitivity:

Assay sensitivity was determined as the concentration at which the antibody preparation produced an assay readout equal to the cut-off value. Because it was so far not possible to purify human anti-β1-AR antibodies sufficiently from patient sera, the assay sensitivity was determined by using the polyclonal goat anti-β1-AR antibody, as described above under item 5.1.1. The cut-off value was determined at approximately 10 nM.

Recovery:

To determine recovery, 20 plasma samples from healthy volunteers were spiked with the rat 13F6 anti β1-AR antibody (that is obtainable from the hybridoma cell (clone) as deposited under DSM ACC3174) in an assay concentration of: 760 nM. All 20 samples showed inhibition values above the cut-off point value with mean coefficients of variation (CV) of 2.54% and therefore completely fulfilled the criteria for recovery.

Precision:

Intra-assay (repeatability) and inter-assay (intermediate precision) variability was evaluated by using a validation sample (VS) and a positive control (PC) both spiked with rat 13F6 antibodies at an assay concentration of 253 nM and 760 nM respectively. Four replicates were used on each plate, which were carried out on three different days. We found a mean intra-assay CV of 4.8% and an inter-assay CV of 16.2% for the VS, and a mean intra-assay CV of 3.6% and an inter-assay CV of 15.4% for the PC, respectively.

Measurement of the 167 human DCM serum samples and 110 age-matched volunteers in three independent measurements resulted in a mean inter-assay CV of 14.4% for the patient group, and of 16.9% for the control group.

Stability:

Storage conditions and blood serum sample stability was investigated for the VS. Storage at either 22° C. for 3 h or at 4° C. for 16 h had no negative impact on the measurement of rat 13F6 anti β1-AR antibodies and resulted in 95.1% and 92.3% recovery compared to the unstressed VS. Also three times repeated freeze/thaw cycles had no influence on the results of the VS.

Additionally, the stability of anti-β1-AR antibody determination was analysed in whole blood samples. Ten DCM samples, which were tested positive for anti-β1-AR antibodies, were stored at 20° C. for 20 h and analysed again. A recovery of 94.7% (SD±10.4) was determined, thus showing a high antibody stability in whole blood comparable to the stability in serum.

5.1.3 Screening Results of Patients Suffering from DCM Vs. Volunteers

The presence of anti-β1-AR antibodies in 167 DCM patients presenting with a left ventricular end-diastolic volume (LV-EF) of <45% and of 110 age-matched volunteers who reported no known heart disease upon blood sampling was analyzed.

The 167 patients suffering from DCM to which reference is made in FIGS. 14(A) and 14(B) is made had been investigated by echocardiography and are characterized by having an ejection fraction of less than 45%. Sera from strictly age-matched subjects (n=110) from a local blood donor bank served as controls. All these control subjects (n=110) had not reported cardiovascular disease upon blood sampling. The mean patient age was 60.9+11.2 years in the volunteer group which did not differ significantly (two-tailed t-test).

By applying the standard parameters identified under item 5.1.2, supra, in the DCM group, 62.2% of these samples were identified to be positive for relevant anti-β1-AR antibodies, and only 8.2% in the age-matched control group (FIG. 14A).

The different prevalences of (false) positive control subjects which are obvious from the comparison of FIGS. 8 and 9 with FIG. 14(A) relate to the fact that the control group of healthy subjects investigated in the study underlying the results shown in FIGS. 7 to 9 were not age-matched compared to the diseased DCM group underlying the result shown in FIG. 14(A). These control subjects presented in FIGS. 7 to 9 were markedly younger than the diseased population.

The data presented in FIG. 14(A) relates to an approach to systematically compare the control group of healthy subjects on an age-matched basis. Many researchers (e.g., Effors B. Stress, immunity and aging. Marcel Dekker, New York, N.Y., 1984) have found that the number of false positive biomarker in the control group of healthy subject markedly increases with increasing age of the investigated control population.

5.1.4 Screening Results of Patients Suffering from ICM Vs. Volunteers

The presence of anti-β1-AR antibodies in patients (n=156) suffering from “ischemic cardiomyopathy” caused by severe coronary artery disease (ICM patients) and of 110 age-matched volunteers who reported no known heart disease upon blood sampling was analyzed.

Patients suffering from “ischemic cardiomyopathy” caused by severe coronary artery disease (ICM patients) used in the study underlying FIG. 14(C) had been investigated by echocardiography and are characterized by having a left ventricular ejection fraction (LV-EF) of less than 45%. Additionally, coronary heart disease had been confirmed by invasive catheter investigation. Subjects with no known heart disease served as controls (healthy volunteers).

By applying the standard parameters identified under items 5.1.1 and 5.1.2, supra, in the ICM group, a significantly larger number of the samples of the ICM group were identified to be positive for relevant anti-β1-AR antibodies compared to the age-matched control group (FIG. 14(C)).

5.1.5 Comparison of the Inhibitory Effect of Unaltered Sera and the Respective Antibody-Depleted Serum Fractions

In order to demonstrate that the inhibition values which were determined in the cell based (cellular) ELISA were actually due to IgG antibodies 20 anti-β1-AR antibody-positive DCM sera were depleted via Protein G Sepharose to eliminate IgG immunoglobulins. The flow-through from each serum sample was collected and analysed in comparison to the load (untreated serum) by cellular ELISA. It has been observed that ELISA-determined anti-β1AR titers disappeared completely in all investigated antibody-depleted samples (nominal mean Inhibition % was reduced from 13.1% to −31.1%; FIG. 15).

5.2 Peptide Based ELISA Assay

A widely used method for determination of auto-anti-β1-adrenergic antibodies in human serum is a peptide-based ELISA assay. In this peptide based ELISA assay system microtiter plates (Nunc microtiter maxisorp plates) were coated with solutions of 10 μg/ml of the 26-meric peptide (His-Trp-Trp-Arg-Ala-Glu-Ser-Asp-Glu-Ala-Arg-Arg-Cys-Tyr-Asn-Asp-Pro-Lys-Cys-Cys-Asp-Phe-Val-Thr-Asn-Arg of SEQ ID NO:17), corresponding to the amino acid sequence (residues 197-222) of the second extracellular loop of the human β1 receptor, in 0.1M Na₂ CO₃ for 1 h at RT. After saturation of the wells with PBS-T (PBS Dulbecco (Cat No. L1820, Biochrom AG)+0.1% TWEEN 20) supplemented with 3% milk powder, human serum from healthy volunteers or from patients with DCM, respectively, were diluted 1:20 in the same buffer (PBS-T+3% milk) and added to the wells. After incubation for 16 h at 4° C., the bounded antibodies were detected by a secondary anti-human IgG antibodies labelled with peroxidase (Dianova, cat. 109-035-088) (diluted 1:5000 in PBS-T+3% milk). Between each step a 3×PBS-T washing procedure were conducted. Afterwards, 100 μl of TMB substrate (3,3′,5,5′-tetramethylbenzidine) solution were dispensed to all wells. The plate was covered and incubated for 10-30 minutes at 20° C. The enzyme reaction was stopped by addition of 100 μL stop solution (1 M sulfuric acid) to all wells. The absorbance was read at 450 nm (reference filter 650 nm). The reduction of colour intensity was directly related to the amount of human β1-receptor antibodies in the sample.

The peptide based ELISA method was conducted to clarify the potential as a diagnostic tool for this ELISA assay. The mean focus was concentrated on the relative performance of healthy volunteers to DCM patients.

Patients suffering from idiopathic dilated cardiomyopathy (DCM patients; n=82) had been investigated by echocardiography and are characterized by having an ejection fraction of less than 45%. Additionally, coronary heart disease had been excluded by invasive catheter investigation. Subjects with no known heart disease served as controls (healthy volunteers; n=43). As it is evident from FIG. 1 in both DCM patients and healthy volunteers (control group), an identical percentage of auto anti-β1-adrenergic receptor antibody (antibodies) were observed.

Changes to lower or higher cut-off ratios did not vary the results significantly (data not shown). This high percentage of antibody-positive healthy subjects was not expected and may be explained by partly false positive determination of auto anti-β1-adrenergic receptor antibody (antibodies). From these findings, the inventors conclude that the peptide conformation after immobilisation onto the microtiter plate probably does not reflect the native structure of the human β1-adrenoceptor EC II loop. The artificial folding of the peptide might produce neo-epitopes which could be responsible for a non-specific antibody binding. In a similar approach, human plasma samples were purified by an affinity column. To this end, the same 26-meric peptide (His-Trp-Trp-Arg-Ala-Glu-Ser-Asp-Glu-Ala-Arg-Arg-Cys-Tyr-Asn-Asp-Pro-Lys-Cys-Cys-Asp-Phe-Val-Thr-Asn-Arg of SEQ ID NO:17) was coupled to CNBr-activated Sepharose 4B (GE Healthcare, cat. 17-0430-01). Purification was done according to manufacturer's instruction. The pre-purified antibody fractions were then analysed in the peptide-based ELISA assay, wherein no significant differences between DCM patients versus healthy volunteers were observed (data not shown).

5.2.1 Peptide Based ELISA Assay

By using the identical serum samples from the 167 DCM patients presenting with an left ventricular end-diastolic volume (LV-EF) of <45% and of 110 age-matched volunteers who reported no known heart disease upon blood sampling and the age matched control group as used in the cell (cellular) based SF9 β1-AR ELISA assays (as described under item 5.1.3, supra).

Nunc microtiter maxisorp plates were coated with 0.5 μg/ml peptide, the 26-meric peptide (His-Trp-Trp-Arg-Ala-Glu-Ser-Asp-Glu-Ala-Arg-Arg-Cys-Tyr-Asn-Asp-Pro-Lys-Cys-Cys-Asp-Phe-Val-Thr-Asn-Arg of SEQ ID NO:17), in 0.1M Na₂CO₃ or buffer alone for 16 h at 4° C. After saturation of the wells with PBS supplemented with 3% milk powder and 0.1% TWEEN 20, human serum from healthy volunteers or from patients with DCM, respectively, were diluted 1:20 in PBS-T+3% BSA+10% FCS and added to the wells. After incubation for 2 h at RT, the bound antibodies were detected by a secondary anti-human IgG antibody labelled with peroxidase, diluted 1:20000 in PBS-T+3% milk. Between each step, plates were washed 3× with PBS-T. Afterwards, 100 μl of TMB substrate (3,3′,5,5′-tetramethylbenzidine) substrate solution were dispensed to all wells. The plate was covered and incubated for 10-30 minutes at RT. The enzyme reaction was stopped by addition of 100 μL stop solution (1 M sulfuric acid) to all wells. The absorbance was read at 450 nm (reference filter 650 nm). The reduction of colour intensity was directly related to the amount of human anti-β1 receptor antibodies in the sample. Strong positivity was defined as 1.5 times the background density.

As shown in FIG. 14B 29.9% anti-β1-AR antibody positive DCM patients versus 35.5% positive findings in the control group were observed.

As explained above under item 5.1.3 in the cell based ELISA assay anti-β1-AR antibodies were detected in about 60% of DCM patients and in about 8% of healthy volunteers (control group) using the same cut-off values (see FIGS. 14A and 14B) by using the identical serum samples from the 167 DCM patients and healthy control group (n=110). Anti-β1-AR antibody-titers (defined as inhibition of β1MAb-binding) were no longer detected after depleting sera from IgG antibodies by protein G adsorption (see FIG. 15). Consequently, the result obtained from the cell based ELISA assay sharply differs from the peptide based ELISA assay conducted with the linear 26-meric peptide derived from the second extracellular β1-AR loop that yielded a high number of false positive results precluding any specific identification of DCM patients.

5.4 Data Analysis

IC₅₀ values were calculated by using standard curve analysis (four parameter logistic′) from Sigma plot software, version 11. All other calculations were performed with EXCEL software, version 2003/2007.

What is claimed is:

1. An antibody or antibody fragment thereof that binds to the second extracellular loop of human β₁-adrenoreceptor.

2. The antibody of claim 1, wherein said second extracellular loop of the human β1-adrenoreceptor comprises the amino acid sequence as depicted in SEQ ID NO: 17.

3. The antibody of claim 1, wherein said antibody is obtainable from a host cell and has a deposit number selected from the group consisting of DSM ACC3121, DSM ACC3174, DSM ACC3175, DSM ACC3176 and DSM ACC3177.

4. The antibody of claim 1, wherein said antibody is obtainable from
(i) a host cell and wherein said antibody comprises at least one antibody light chain variable region having the CDR sequences CDRL1 to CDRL3 of SEQ ID NOs: 10 to 12, respectively; and at least one antibody heavy chain variable region having the CDR sequences CDRH1 to CDRH3 of SEQ ID NOs: 7 to 9, respectively;
(ii) a host cell and wherein said antibody comprises at least one antibody light chain variable region having the CDR sequences CDRL1 to CDRL3 of SEQ ID NOs: 34 to 36, respectively; and at least one antibody heavy chain variable region having the CDR sequences CDRH1 to CDRH3 of SEQ ID NOs: 37 to 39, respectively;
(iii) a host cell and wherein said antibody comprises at least one antibody light chain variable region having the CDR sequences CDRL1 to CDRL3 of SEQ ID NOs: 44 to 46, respectively; and at least one antibody heavy chain variable region having the CDR sequences CDRH1 to CDRH3 of SEQ ID NOs: 47 to 49, respectively;
(iv) a host cell and wherein said antibody comprises at least one antibody light chain variable region having the CDR sequences CDRL1 to CDRL3 of SEQ ID NOs: 54 to 56, respectively; and at least one antibody heavy chain variable region having the CDR sequences CDRH1 to CDRH3 of SEQ ID NOs: 57 to 59, respectively; or
(v) a host cell and wherein said antibody comprises at least one antibody light chain variable region having the CDR sequences CDRL1 to CDRL3 of SEQ ID NOs: 64 to 66, respectively; and at least one antibody heavy chain variable region having the CDR sequences CDRH1 to CDRH3 of SEQ ID NOs: 67 to 69, respectively.

5. The antibody of claim 1, wherein said antibody is obtainable from
(i) a host cell and wherein said antibody comprises a light chain variable region comprising the sequence of SEQ ID NO: 6 having up to ten conservative amino acid substitutions and a heavy chain variable region comprising the sequence of SEQ ID NO: 4 having up to ten conservative amino acid substitutions;
(ii) a host cell and wherein said antibody comprises a light chain variable region comprising the sequence of SEQ ID NO: 31 having up to ten conservative amino acid substitutions and a heavy chain variable region comprising the sequence of SEQ ID NO: 33 having up to ten conservative amino acid substitutions;
(iii) a host cell and wherein said antibody comprises a light chain variable region comprising the sequence of SEQ ID NO: 41 having up to ten conservative amino acid substitutions and a heavy chain variable region comprising the sequence of SEQ ID NO: 43 having up to ten conservative amino acid substitutions;
(iv) a host cell and wherein said antibody comprises a light chain variable region comprising the sequence of SEQ ID NO: 51 having up to ten conservative amino acid substitutions and a heavy chain variable region comprising the sequence of SEQ ID NO: 53 having up to ten conservative amino acid substitutions; or
(v) a host cell and wherein said antibody comprises a light chain variable region comprising the sequence of SEQ ID NO: 61 having up to ten conservative amino acid substitutions and a heavy chain variable region comprising the sequence of SEQ ID NO: 63 having up to ten conservative amino acid substitutions.

6. The antibody of claim 1, wherein said antibody is obtainable from
(i) a host cell and wherein said antibody comprises a light chain variable region that comprises SEQ ID NO: 6 and a heavy chain variable region that comprises SEQ ID NO: 4;
(ii) a host cell and wherein said antibody comprises a light chain variable region that comprises SEQ ID NO: 31 and a heavy chain variable region that comprises SEQ ID NO: 33;
(iii) a host cell and wherein said antibody comprises a light chain variable that comprises SEQ ID NO: 41 and a heavy chain variable region that comprises SEQ ID NO: 43;
(iv) a host cell and wherein said antibody comprises a light chain variable region that comprises SEQ ID NO: 51 and a heavy chain variable region that comprises SEQ ID NO: 53; or
(v) a host cell and wherein said antibody comprises a light chain variable region that comprises SEQ ID NO: 61 and a heavy chain variable region that comprises SEQ ID NO: 63.

7. An antibody which binds to the same epitope as the antibody of claim 2.

8. The antibody of claim 1, wherein said antibody is a chimeric, humanized, bispecific or fully-human antibody.

9. The antibody of claim 1, wherein said antibody is a monoclonal or polyclonal antibody.

10. The antibody of claim 1, wherein said antibody is a human, murine or rat monoclonal antibody.

11. The antibody of claim 1, wherein the antibody is obtainable from a host cell with a deposit number selected from the group consisting of DSM ACC3121, DSM ACC3175, DSM ACC3176 and DSM ACC3177 and wherein said antibody is a murine monoclonal antibody.

12. The antibody of claim 1, wherein the antibody is obtainable from a host cell with a deposit number DSM ACC3174 and wherein said antibody is a rat monoclonal antibody.

13. The antibody of claim 1, wherein the antibody is obtainable from a host cell with a deposit number DSM ACC3121 and wherein said antibody further comprises a heavy chain constant region, wherein the heavy chain constant region comprises a γ1, γ2a, γ2b, or γ3 mouse heavy chain constant region or a variant thereof.

14. The antibody of claim 1, wherein the antibody is obtainable from a host cell with a deposit number DSM ACC3121 and wherein said antibody further comprises a light chain constant region, wherein the light chain constant region comprises a kappa or lambda light chain constant region.

15. The antibody of claim 1, wherein the antibody is an antibody fragment selected from the group consisting of Fab, Fab′, Fab′-SH, Fv, scFv, F(ab′)₂ and a diabody.

16. The antibody of claim 1, wherein the antibody is obtainable from a host cell with a deposit number DSM ACC3121 and wherein said antibody has at least one of the following properties:
(a) the antibody binds to the second extracellular loop of the human β₁-adrenoreceptor with an equilibrium dissociation constant (K_{d}) of 1000 pM or less;
(b) the binding affinity to the second extracellular loop of the human β1-adrenoreceptor is competitively inhibited with an 1050 value of 2000 pM or less in the presence of the peptide cyclo (Ala-Asp-Glu-Ala-Arg-Arg-Cys-Tyr-Asn-Asp-Pro-Lys-Cys-Ser-Asp-Phe-Val-Gln); and
(c) the binds to the second extracellular loop of the human β₁-adrenoreceptor with an affinity (K_{d}) that is at least 10-fold lower compared to rat monoclonal antibodies, preferably to the rat monoclonal antibody that is obtainable from the host cell with the deposit number DSM ACC3174, or goat polyclonal antibodies that bind to the second extracellular loop of the human β₁-adrenoreceptor.

17. The antibody of claim 16, wherein said antibody is obtainable from the host cell with the deposit number DSM ACC3121 and wherein said antibody binds to the second extracellular loop of the human β₁-adrenoreceptor with an equilibrium dissociation constant (K_{d}) of 510 pM or less.

18. A nucleic acid molecule encoding the antibody of claim 1.

19. A vector comprising the nucleic acid of claim 18.

20. A host cell comprising at least one of the nucleic acid molecule of claim 18 and the vector comprising the nucleic acid molecule of claim 18.

21. The host cell of claim 20, wherein the host cell is a hybridoma.

22. The host cell of claim 21, wherein the hybridoma is selected from the group consisting of a host cell of deposit number DSM ACC3121, DSM ACC3174, DSM ACC3175, DSM ACC3176 and DSM ACC3177.

23. A method for producing the antibody of claim 1, wherein said method comprises culturing a host cell of claim 20 and recovering said antibody from said culture.

24. The method of claim 23, wherein the antibody comprises an antibody that binds to the second extracellular loop of the human β₁-adrenoreceptor.

25. A method for identifying a patient having or being at risk of developing a disease associated with human β₁-adrenoreceptor, comprising the steps of:
(a) contacting a human β₁-adrenoreceptor with a biological sample of said patient, thereby allowing molecule(s) or compound(s) contained in said biological sample to bind to the human β₁-adrenoreceptor;
(b) contacting the human β₁-adrenoreceptor of (a) with the antibody or antibody fragment thereof of claim 1, thereby allowing said antibody or antibody fragment thereof to bind to human β₁-adrenoreceptor which is not bound by molecule(s) or compound(s) contained in said biological sample of (a);
(c) contacting a human β₁-adrenoreceptor which was not contacted with said biological sample of (a) with the antibody or antibody fragment thereof of claim 1, thereby allowing said antibody or antibody fragment thereof to bind to said human β₁-adrenoreceptor which was not contacted with said biological sample of (a);
(d) measuring
(i) a binding signal between the human β₁-adrenoreceptor and the antibody of step (b), and
(ii) a binding signal between the human β₁-adrenoreceptor and the antibody of step (c); and
(e) comparing the binding signal measured in (d)(i) with that of (d)(ii),
wherein a binding signal measured in (d)(i) which is at least 40% lower than that measured in (d)(ii) indicates that said patient has or is at risk of developing said disease.

26. The method of claim 25, wherein said antibody is an antibody that is obtainable from a host cell with a deposit number selected from the group consisting of DSM ACC3121, DSM ACC3174, DSM ACC3175, DSM ACC3176 and DSM ACC3177.

27. The method of claim 25, wherein said antibody is the antibody that is obtainable from a host cell with a deposit number DSM ACC3121.

28. The method of claim 25 of claims, wherein the human β₁-adrenoreceptor is immobilized on a solid phase prior to contacting the human β₁-adrenoreceptor with a biological sample or the antibody.

29. The method of claim 25, wherein the solid phase is a material selected from the group consisting of poly-L-Lysin, poly-D-Lysin precoated, sepharose, latex, glass, polystyrene, polyvinyl, nitrocellulose and silicon.

30. The method of claim 25, wherein the disease is selected from the group consisting of heart diseases, comprising idiopathic dilated cardiomyopathy (DCM), ischaemic cardiomyopathy (ICM), Chagas disease, infectious and non-infectious heart disease, ischemic and non-ischemic heart disease, inflammatory heart disease and myocarditis, cardiac dilatation, idiopathic cardio-myopathy, immune-cardiomyopathy, heart failure, and any cardiac arrhythmia including ventricular and supraventricular premature capture beats.

31. The method of claim 30, wherein the disease is idiopathic dilated cardiomyopathy (DCM).

32. The method of claim 30, wherein the disease is ischemic cardiomyopathy (ICM).

33. The method of claim 25, wherein the measuring of the binding signal comprises measuring a label associated with the antibody of claim 1.

34. The method of claim 32, wherein labelling antibody comprises at least one of a system emitting a signal or wherein the antibody is recognized by (a) at least a second molecule(s) or compound(s) comprising a system emitting a signal.

35. The method of claim 33, wherein the system emitting the signal comprises an enzyme emitting the signal.

36. The method of claim 33, wherein the at least second molecule(s) or compound(s) are selected from the group consisting of peptides, polypeptides, low-molecular substances, antibodies or fragments or derivates thereof

37. The method of any one of claim 24, wherein the method is an ELISA, an EIA or a RIA.

38. A diagnostic agent comprising an antibody of claim 1 for the detection of molecule(s) or compound(s) in biological sample of a subject.

39. The diagnostic agent of claim 37, wherein said antibody is obtained from a host cell with a deposit number DSM ACC3121.

40. The method of claim 25, wherein the biological sample comprises blood, plasma or serum.

41. The method of claim 40, wherein said biological sample comprises molecule(s) or compound(s) selected from the group consisting of antibodies, proteins, protein-fragments, peptides, amino acids and/or derivates thereof.

42. The method of claim 41, wherein said molecule(s) or compound(s) are antibodies.

43. The method of claim 42, wherein said antibodies are auto anti-β1-adrenergic antibodies.

44. A diagnostic kit for the detection of auto anti-β1-adrenergic receptor antibodies comprising the antibodies of claim 1, and at least one of a host cell and a diagnostic agent.


SEQUENCE LISTING (69 sequences)

SEQ ID NO: 1 - DNA, 1434 bp, Homo sapiens
atgggcgcgggggtgctcgtcctgggcgcctccgagcccggtaacctgtcgtcggccgcaccgctccccgacggcgcggccaccgcggcgcggctgctggtgcccgcgtcgccgcccgcctcgttgctgcctcccgccagcgaaagccccgagccgctgtctcagcagtggacagcgggcatgggtctgctgatggcgctcatcgtgctgctcatcgtggcgggcaatgtgctggtgatcgtggccatcgccaagacgccgcggctgcagacgctcaccaacctcttcatcatgtccctggccagcgccgacctggtcatggggctgctggtggtgccgttcggggccaccatcgtggtgtggggccgctgggagtacggctccttcttctgcgagctgtggacctcagtggacgtgctgtgcgtgacggccagcatcgagaccctgtgtgtcattgccctggaccgctacctcgccatcacctcgcccttccgctaccagagcctgctgacgcgcgcgcgggcgcggggcctcgtgtgcaccgtgtgggccatctcggccctggtgtccttcctgcccatcctcatgcactggtggcgggcggagagcgacgaggcgcgccgctgctacaacgaccccaagtgctgcgacttcgtcaccaaccgggcctacgccatcgcctcgtccgtagtctccttctacgtgcccctgtgcatcatggccttcgtgtacctgcgggtgttccgcgaggcccagaagcaggtgaagaagatcgacagctgcgagcgccgtttcctcggcggcccagcgcggccgccctcgccctcgccctcgcccgtccccgcgcccgcgccgccgcccggacccccgcgccccgccgccgccgccgccaccgccccgctggccaacgggcgtgcgggtaagcggcggccctcgcgcctcgtggccctgcgcgagcagaaggcgctcaagacgctgggcatcatcatgggcgtcttcacgctctgctggctgcccttcttcctggccaacgtggtgaaggccttccaccgcgagctggtgcccgaccgcctcttcgtcttcttcaactggctgggctacgccaactcggccttcaaccccatcatctactgccgcagccccgacttccgcaaggccttccagggactgctctgctgcgcgcgcagggctgcccgccggcgccacgcgacccacggagaccggccgcgcgcctcgggctgtctggcccggcccggacccccgccatcgcccggggccgcctcggacgacgacgacgacgatgtcgtcggggccacgccgcccgcgcgcctgctggagccctgggccggctgcaacggcggggcggcggcggacagcgactcgagcctggacgagccgtgccgccccggcttcgcctcggaatccaaggtgtag

SEQ ID NO: 2 - protein, 477 aa, Homo sapiens
MGAGVLVLGASEPGNLSSAAPLPDGAATAARLLVPASPPASLLPPASESPEPLSQQWTAGMGLLMALIVLLIVAGNVLVIVAIAKTPRLQTLTNLFIMSLASADLVMGLLVVPFGATIVVWGRWEYGSFFCELWTSVDVLCVTASIETLCVIALDRYLAITSPFRYQSLLTRARARGLVCTVWAISALVSFLPILMHWWRAESDEARRCYNDPKCCDFVTNRAYAIASSVVSFYVPLCIMAFVYLRVFREAQKQVKKIDSCERRFLGGPARPPSPSPSPVPAPAPPPGPPRPAAAAATAPLANGRAGKRRPSRLVALREQKALKTLGIIMGVFTLCWLPFFLANVVKAFHRELVPDRLFVFFNWLGYANSAFNPIIYCRSPDFRKAFQGLLCCARRAARRRHATHGDRPRASGCLARPGPPPSPGAASDDDDDDVVGATPPARLLEPWAGCNGGAAADSDSSLDEPCRPGFASESKV

SEQ ID NO: 3 - DNA, 315 bp, Mus musculus
ctggtgaagcctggggcttcagtgaagatatcctgcaaggcttctggttactcattcactgactactacatgcactgggtgaagcaaagccatgtaaagagccttgagtggattggacgtattaatccttacagtggtgctcctagctacacccagaatttcaaggccaaggccagcttgactgtagataagtcctccagcacagcctacatggagctccacagcctgacatctgaggactctgcagtctattactgcgcaagagctaactgggacgggtactttgactactggggccaaggcaccactctcaca

SEQ ID NO: 4 - protein, 104 aa, Mus musculus
LVKPGASVKICKASGYSFTDYYMHWVKQSHVKSLEWIGRINPYSGAPSYTQNFKAKASLTVDKSSSTAYMELHSLTSEDSAVYYCARANWDGYFDYWGQGTTLT

SEQ ID NO: 5 - DNA, 291 bp, Mus musculus
atgtctgcatctccaggggagaaggtcaccatgacctgcagtgccagctcaagtgtaagttacatgtactggtaccagcagaagtcaggcacctcccccaaaagatggatttatgacacatccaaattggcttctggagtccctgttcgcttcagtggcagtgggtctgggacctcttactctctcacaatcagcagcatggaggctgaagatgctgccacttattactgccagcagtggagtagtaacccatggacgttcggtggaggcaccaaattggaaatcaaacgg

SEQ ID NO: 6 - protein, 97 aa, Mus musculus
MSASPGEKVTMTCSASSSVSYMYWYQQKSGTSPKRWIYDTSKLASGVPVRFSGSGSGTSYSLTISSMEAEDAATYYCQQWSSNPWTFGGGTKLEIKR

SEQ ID NO: 7 - protein, 5 aa, Mus musculus
DYYMH

SEQ ID NO: 8 - protein, 17 aa, Mus musculus
RINPYSGAPSYTQNFKA

SEQ ID NO: 9 - protein, 9 aa, Mus musculus
ANWDGYFDY

SEQ ID NO: 10 - protein, 10 aa, Mus musculus
SASSSVSYMY

SEQ ID NO: 11 - protein, 7 aa, Mus musculus
DTSKLAS

SEQ ID NO: 12 - protein, 9 aa, Mus musculus
QQWSSNPWT

SEQ ID NO: 13 - protein, 22 aa, Artificial Sequence
  Amino acid sequence homologous to an ECII epitope of human BETA1-AR
RAESDEARRCYNDPKCXDFVTG

SEQ ID NO: 14 - protein, 18 aa, Artificial Sequence
  Amino acid sequence homologous to an ECII epitope of human BETA1-AR
ADEARRCYNDPKCXDFVQ

SEQ ID NO: 15 - protein, 25 aa, Artificial Sequence
  Amino acid sequence homologous to an ECII epitope of human BETA1-AR
ARAESDEARRCYNDPKCXDFVTNRQ

SEQ ID NO: 16 - protein, 18 aa, Artificial Sequence
  Amino acid sequence homologous to an ECII epitope of human BETA1-AR
ADEARRCYNDPKCSDFVQ

SEQ ID NO: 17 - protein, 26 aa, Artificial Sequence
  Amino acid sequence homologous to an ECII epitope of human BETA1-AR
HWWRAESDEARRCYNDPKCCDFVTNR

SEQ ID NO: 18 - protein, 25 aa, Artificial Sequence
  Amino acid sequence homologous to an ECII epitope of human BETA1-AR
ARAESDEARRCYNDPKCCDFVTNRQ

SEQ ID NO: 19 - protein, 23 aa, Artificial Sequence
  Amino acid sequence homologous to an ECII epitope of human BETA1-AR
RAESDEARRCYNDPKCCDFVTNR

SEQ ID NO: 20 - DNA, 20 bp, Artificial Sequence
  Oligonuclotide primer
ggcatcctcaccctgaagta

SEQ ID NO: 21 - DNA, 20 bp, Artificial Sequence
  Oligonuclotide primer
gtcaggcagctcgtagctct

SEQ ID NO: 22 - DNA, 32 bp, Artificial Sequence
  Oligonuclotide primer
ggtgatatcgtgctcacccaatctccagcaat

SEQ ID NO: 23 - DNA, 32 bp, Artificial Sequence
  Oligonuclotide primer
gggaagatggatccagttggtgcagcatcagc

SEQ ID NO: 24 - DNA, 36 bp, Artificial Sequence
  Oligonuclotide primer
gaggtgaagctgcaggagtcaggacctagcctggtg

SEQ ID NO: 25 - DNA, 22 bp, Artificial Sequence
  Oligonuclotide primer
aggtccagctgcagcagtgagg

SEQ ID NO: 26 - DNA, 38 bp, Artificial Sequence
  Oligonuclotide primer
ccaggggccagtggatagacaagcttgggtgtcgtttt

SEQ ID NO: 27 - DNA, 19 bp, Artificial Sequence
  Oligonuclotide primer
gggaagatggatccagttg

SEQ ID NO: 28 - DNA, 18 bp, Artificial Sequence
  Oligonuclotide primer
caggggccagtggataga

SEQ ID NO: 29 - DNA, 32 bp, Artificial Sequence
  Oligonucleotide primer
ggtgatatcatgatgacccaaactccactctc

SEQ ID NO: 30 - DNA, 309 bp, Rattus norvegicus
ctgcttatcagccttggaggtcaagcctccatctcttgccggtctagtcagagcctgatacacagtaatggaaacacctactttgattggttcctgcagaagccaggccagtctccacagctcctcatctatcaggtttccaacagattttctggggtcccagacaggttcagtggtagtgggtcagggacggatttcaccctcaagatcagcagattagagcctgaggacttgggagtttattactgcttacaacgtacacatgatccgtacacgtttggagctgggaccaagctggaactgaaacgg

SEQ ID NO: 31 - protein, 103 aa, Rattus norvegicus
LLISLGGQASISCRSSQSLIHSNGNTYFDWFLQKPGQSPQLLIYQVSNRFSGVPDRFSGSGSGTDFTLKISRLEPEDLGVYYCLQRTHDPYTFGAGTKLELKR

SEQ ID NO: 32 - DNA, 315 bp, Rattus norvegicus
ttactgcagcctggaaggtccatgaaactctcctgtgcagcctcaggattcactttcagtaactatggcatggcctgggtccgccaggctccaaagaagggtctggactgggtcgcatatattagttatgatggtggtggcacttactatcgcgactccgtgaagggccgattcactgtctccagagataatgcaaaaagcaccctatacctgctaatggacagtctgaggtctgaggacacggccacttattactgtacaaccgggaactttgattactggggccaaggagtcatggtcacggtctcctcagct

SEQ ID NO: 33 - protein, 105 aa, Rattus norvegicus
LLQPGRSMKLSCAASGFTFSNYGMAWVRQAPKKGLDWVAYISYDGGGTYYRDSVKGRFTVSRDNAKSTLYLLMDSLRSEDTATYYCTTGNFDYWGQGVMVTVSSA

SEQ ID NO: 34 - protein, 16 aa, Rattus norvegicus
RSSQSLIHSNGNTYFD

SEQ ID NO: 35 - protein, 7 aa, Rattus norvegicus
QVSNRFS

SEQ ID NO: 36 - protein, 9 aa, Rattus norvegicus
LQRTHDPYT

SEQ ID NO: 37 - protein, 5 aa, Rattus norvegicus
NYGMA

SEQ ID NO: 38 - protein, 17 aa, Rattus norvegicus
YISYDGGGTYYRDSVKG

SEQ ID NO: 39 - protein, 5 aa, Rattus norvegicus
GNFDY

SEQ ID NO: 40 - DNA, 279 bp, Mus musculus
atgtctgcatctccaggggagaaggtcaccatgacctgcagtgccagctcaagtgtaagttacatgtattggtatcagcagaagtcaggcacctcccccagaagatggatttatgacacatccaaactggcttctggagtccctgctcgcttcagtggcagtgggtctgggacctcttactctctcacaatcagcagcatggaggctgaagatgttgccacttattactgccagcagtggagtactaacccatggacgttcggtggaggcaccaagctg

SEQ ID NO: 41 - protein, 93 aa, Mus musculus
MSASPGEKVTMTCSASSSVSYMYWYQQKSGTSPRRWIYDTSKLASGVPARFSGSGSGTSYSLTISSMEAEDVATYYCQQWSTNPWTFGGGTKL

SEQ ID NO: 42 - DNA, 324 bp, Mus musculus
ctggtgaagcctggggcttcagtgaggatatcctgcaaggcttctggttactcattcactgactactacatacactgggtgaaacaaagccatgtaaagagccttgagtggattggacgtattaatccttacaatggtgctactaactacaaccagaatttcaaggacaaggccaacttgactgtagataagtcctccagcacagcctacatggagctccacagcctgacatctgaggactctgcagtctatttctgtgcaagagctaactgggacggttactttgacgactggggtcaaggcaccactctcacagtctcctca

SEQ ID NO: 43 - protein, 108 aa, Mus musculus
LVKPGASVRISCKASGYSFTDYYIHWVKQSHVKSLEWIGRINPYNGATNYNQNFKDKANLTVDKSSSTAYMELHSLTSEDSAVYFCARANWDGYFDDWGQGTTLTVSS

SEQ ID NO: 44 - protein, 10 aa, Mus musculus
SASSSVSYMY

SEQ ID NO: 45 - protein, 7 aa, Mus musculus
DTSKLAS

SEQ ID NO: 46 - protein, 9 aa, Mus musculus
QQWSTNPWT

SEQ ID NO: 47 - protein, 5 aa, Mus musculus
DYYIH

SEQ ID NO: 48 - protein, 17 aa, Mus musculus
RINPYNGATNYNQNFKD

SEQ ID NO: 49 - protein, 9 aa, Mus musculus
ANWDGYFDD

SEQ ID NO: 50 - DNA, 279 bp, Mus musculus
atgtctgcatctccaggggagaaggtcaccatgacctgcagagccagctcaagtgtaagttacatgtactggtaccagcagaagtcaggcacctcccccaaaagatggatttatgacacatccaaactggcttctggagtccctgctcgcttcagtggcagtgggtctgggacctcttactctctcacaatcagcagcatggaggctgaagatgctgccacttattactgccagcagtggagtagtaacccatggacgttcggtggaggcaccaagctg

SEQ ID NO: 51 - protein, 93 aa, Mus musculus
MSASPGEKVTMTCRASSSVSYMYWYQQKSGTSPKRWIYDTSKLASGVPARFSGSGSGTSYSLTISSMEAEDAATYYCQQWSSNPWTFGGGTKL

SEQ ID NO: 52 - DNA, 324 bp, Mus musculus
ctggtgaagcctggggcttcagtgaagatatcctgcaaggcttctggttactcattcactgactactacatgcactgggtgaagcaaagccatgtaaagagccttgaatggattggacgtattaatccttacaatggtgctactaacttcaaccagaatttcatggacaaggccagcttgcctgtagttaagtcctccagcacagcctacctggacctccacagcctgacatctgaggactctgcagtctattactgtgcaagagctaactgggacgggttctttgactactggggccaaggcaccactctcaccgtctcctca

SEQ ID NO: 53 - protein, 108 aa, Mus musculus
LVKPGASVKISCKASGYSFTDYYMHWVKQSHVKSLEWIGRINPYNGATNFNQNFMDKASLPVVKSSSTAYLDLHSLTSEDSAVYYCARANWDGFFDYWGQGTTLTVSS

SEQ ID NO: 54 - protein, 10 aa, Mus musculus
RASSSVSYMY

SEQ ID NO: 55 - protein, 7 aa, Mus musculus
DTSKLAS

SEQ ID NO: 56 - protein, 9 aa, Mus musculus
QQWSSNPWT

SEQ ID NO: 57 - protein, 5 aa, Mus musculus
DYYMH

SEQ ID NO: 58 - protein, 17 aa, Mus musculus
RINPYNGATNFNQNFMD

SEQ ID NO: 59 - protein, 9 aa, Mus musculus
ANWDGFFDY

SEQ ID NO: 60 - DNA, 279 bp, Mus musculus; identical to SEQ ID NO: 50

SEQ ID NO: 61 - protein, 93 aa, Mus musculus
MSASPGEKVTMTCSASSSVSYMHWYQQKSGTSPKRWIYDTSKLASGVPARFSGSGSGTSYSLTISSMEAEDAATYYCQQWSSNPYTFGGGTKL

SEQ ID NO: 62 - DNA, 321 bp, Mus musculus
ctggtgaagcctggggcttcagtgaagatatcctgcaaggcttctggttactcattcactgactactacatgcactgggtgaaacaaagccatgttaagagccttgagtggattggacgtgttaatcctttcaatggtgctactaactacgaccagaatttcaaggccaaggccagcttgactgtagataagtcctccagcacagcccacatggagctccacagcctgacatctgaggactctgctgtctattactgtgcaagaggagattacgacggggtctttacttactggggccaagggactctggtcactgtctct

SEQ ID NO: 63 - protein, 107 aa, Mus musculus
LVKPGASVKISCKASGYSFTDYYMHWVKQSHVKSLEWIGRVNPFNGATNYDQNFKAKASLTVDKSSSTAHMELHSLTSEDSAVYYCARGDYDGVFTYWGQGTLVTVS

SEQ ID NO: 64 - protein, 10 aa, Mus musculus
SASSSVSYMH

SEQ ID NO: 65 - protein, 7 aa, Mus musculus
DTSKLAS

SEQ ID NO: 66 - protein, 9 aa, Mus musculus
QQWSSNPYT

SEQ ID NO: 67 - protein, 5 aa, Mus musculus
DYYMH

SEQ ID NO: 68 - protein, 17 aa, Mus musculus
RVNPFNGATNYDQNFKA

SEQ ID NO: 69 - protein, 9 aa, Mus musculus
GDYDGVFTY